SEQUENCE LISTING (33 sequences)

SEQ ID NO: 1 - DNA, 1275 bp, Artificial Sequence
  synthetic sequence of CA-DUX4
atggcattgcctacaccttcagactctacgctgcctgcagaggctaggggaagaggtagacggcggcgattggtgtggactccatcacaatccgaagctcttcgcgcatgcttcgagcgcaatccctatccggggattgccacaagggagaggcttgcacaggctatcggaatcccggaaccgagagtgcagatctggttccaaaatgaacgctctcggcagctcagacagcatcgcagggagtcccgcccgtggccaggaagaaggggaccacctgaaggaagaagaaaacgcacagcggtgactggcagccaaacggctctgctgctccgcgctttcgagaaagatcggttccccggaattgccgcacgcgaagaactcgccagagaaactgggctcccagaatcacgaatacagatttggttccagaaccgcagagcaagacacccaggccaggggggacgggcacctgctcaggccggtggactctgctctgctgcccctgggggcggccatccagcaccttcctgggtggctttcgctcatactggcgcttggggtaccgggctgcctgctccgcatgttccctgtgctccaggggccctcccgcagggagcgtttgtttcccaggcagctagggctgcacctgccctgcaaccatcacaggcagcgccagctgaaggcatcagccaacccgccccagcccgcggagattttgcttatgcagcgccagcacctccagacggtgccctgagccacccccaagcccccagatggccccctcaccctggtaagtcccgggaagaccgcgatccccaacgagatggactgcccggtccttgcgctgtggcccagccaggacctgctcaagccggccctcaggggcaaggagtgctggccccacctacaagccagggatctccctggtggggttggggacgcggacctcaggttgctggagccgcttgggagcctcaggccggagctgcaccgccgccacaaccggcccctcccgacgcgtcagcgtccgcccgacaaggccagatgcagggaatcccagcacctagccaagctcttcaagagcctgccccttggagcgcactgccgtgtgggctgctcctggatgaactcctggctagcccagaatttctccagcaggcacagccactcctggaaacagaagctccgggagagctcgaagcctccgaagaagcagcaagcctggaggcacctctttccgaggaggagtatagagcccttctggaagaactttga

SEQ ID NO: 2 - protein, 689 aa, Artificial Sequence [sequence omitted]

SEQ ID NO: 3 - protein, 424 aa, Artificial Sequence
  synthetic sequence of NCBI Reference Sequence - NP_001280727.1
MALPTPSDSTLPAEARGRGRRRRLVWTPSQSEALRACFERNPYPGIATRERLAQAIGIPEPRVQIWFQNERSRQLRQHRRESRPWPGRRGPPEGRRKRTAVTGSQTALLLRAFEKDRFPGIAAREELARETGLPESRIQIWFQNRRARHPGQGGRAPAQAGGLCSAAPGGGHPAPSWVAFAHTGAWGTGLPAPHVPCAPGALPQGAFVSQAARAAPALQPSQAAPAEGISQPAPARGDFAYAAPAPPDGALSHPQAPRWPPHPGKSREDRDPQRDGLPGPCAVAQPGPAQAGPQGQGVLAPPTSQGSPWWGWGRGPQVAGAAWEPQAGAAPPPQPAPPDASASARQGQMQGIPAPSQALQEPAPWSALPCGLLLDELLASPEFLQQAQPLLETEAPGELEASEEAASLEAPLSEEEYRALLEEL

SEQ ID NO: 4 - protein, 485 aa, Artificial Sequence
  synthetic sequence of GenBank - ACP30489.1
MKGWSLPACGPLQGRLAGWLAVRAGLLAAPAAVHSPAEVHGSPPASLCPRPSVKFRPGLTAMALPTPSDSTLPAEARGRGRRRRLVWTPSQSEALRACFERNPYPGIATRERLAQAIGIPEPRVQIWFQNERSRQLRQHRRESRPWPGRRGPPEGRRKRTAVTGSQTALLLRAFEKDRFPGIAAREELARETGLPESRIQIWFQNRRARHPGQGGRAPAQAGGLCSAAPGGGHPAPSWVAFAHTGAWGTGLPAPHVPCAPGALPQGAFVSQAARAAPALQPSQAAPAEGISQPAPARGDFAYAAPAPPDGALSHPQAPRWPPHPGKSREDRDPQRDGLPGPCAVAQPGPAQAGPQGQGVLAPPTSQGSPWWGWGRGPQVAGAAWEPQAGAAPPPQPAPPDASASARQGQMQGIPAPSQALQEPAPWSALPCGLLLDELLASPEFLQQAQPLLETEAPGELEASEEAASLEAPLSEEEYRALLEEL

SEQ ID NO: 5 - protein, 424 aa, Artificial Sequence
  synthetic sequence of UniProtKB_Swiss-Prot - P0CJ85.1
MALPTPSDSTLPAEARGRGRRRRLVWTPSQSEALRACFERNPYPGIATRERLAQAIGIPEPRVQIWFQNERSRQLRQHRRESRPWPGRRGPPEGRRKRTAVTGSQTALLLRAFEKDRFPGIAAREELARETGLPESRIQIWFQNRRARHPGQGGRAPAQAGGLCSAAPGGGHPAPSWVAFAHTGAWGTGLPAPHVPCAPGALPQGAFVSQAARAAPALQPSQAAPAEGVSQPAPARGDFAYAAPAPPDGALSHPQAPRWPPHPGKSREDRDPQRDGLPGPCAVAQPGPAQAGPQGQGVLAPPTSQGSPWWGWGRGPQVAGAAWEPQAGAAPPPQPAPPDASASARQGQMQGIPAPSQALQEPAPWSALPCGLLLDELLASPEFLQQAQPLLETEAPGELEASEEAASLEAPLSEEEYRALLEEL

SEQ ID NO: 6 - protein, 424 aa, Artificial Sequence [sequence omitted]

SEQ ID NO: 7 - protein, 424 aa, Artificial Sequence [sequence omitted]

SEQ ID NO: 8 - protein, 633 aa, Artificial Sequence
  synthetic sequence of GenBank - ACN62210.1
MNESHTPACARPSRQRRRPQALPPSRREGLAHSRLPRRFSSTLPSTLPGWFLPAADAWEPRERLPVPAGSLERSGEPAPETRPPPPSPLSPFLFVSPAPPPPPPPRPPPPPPPPTTTTTTTPPAGPRPRRPGSLPGWGGLSQGGSPPFMKGWSLPACGPLQGRLAGWLAVRAGLLAAPAAVHSPAEVHGSPPASLCPRPSVKFRPGLTAMALPTPSDSTLPAEARGRGRRRRLVWTPSQSEALRACFERNPYPGIATRERLAQAIGIPEPRVQIWFQNERSRQLRQHRRESRPWPGRRGPPEGRRKRTAVTGSQTALLLRAFEKDRFPGIAAREELARETGLPESRIQIWFQNRRARHPGQGGRAPAQAGGLCSAAPGGGHPAPSWVAFAHTGAWGTGLPAPHVPCAPGALPQGAFVSQAARAAPALQPSQAAPAEGISQPAPARGDFAYAAPAPPDGALSHPQAPRWPPHPGKSREDRDPQRDGLPGPCAVAQPGPAQAGPQGQGVLAPPTSQGSPWWGWGRGPQVAGAAWEPQAGAAPPPQPAPPDASASARQGQMQGIPAPSQALQEPAPWSALPCGLLLDELLASPEFLQQAQPLLETEAPGELEASEEAASLEAPLSEEEYRALLEEL

SEQ ID NO: 9 - protein, 424 aa, Artificial Sequence
  synthetic sequence of GenBank - ADK24706.1
  misc_feature (124)..(124): Xaa can be any naturally occurring amino acid
MALPTPSDSTLPAEARGRGRRRRLVWTPSQSEALRACFERNPYPGIATRERLAQAIGIPEPRVQIWFQNERSRQLRQHRRESRPWPGRRGPPEGRRKRTAVTGSQTALLLRAFEKDRFPGIAAXEELARETGLPESRIQIWFQNRRARHPGQGGRAPAQAGGLCSAAPGGGHPAPSWVAFAHTGAWGTGLPAPHVPCAPGALPQGAFVSQAARAAPALQPSQAAPAEGISQPAPARGDFAYAAPAPPDGALSHPQAPRWPPHPGKSREDRDPQRDGLPGPCAVAQPGPAQAGPQGQGVLAPPTSQGSPWWGWGRGPQVAGAAWEPQAGAAPPPQPAPPDASASARQGQMQGIPAPSQALQEPAPWSALPCGLLLDELLASPEFLQQAQPLLETEAPGELEASEEAASLEAPLSEEEYRALLEEL

SEQ ID NO: 10 - protein, 424 aa, Artificial Sequence [sequence omitted]

SEQ ID NO: 11 - protein, 424 aa, Artificial Sequence [sequence omitted]

SEQ ID NO: 12 - protein, 424 aa, Artificial Sequence [sequence omitted]

SEQ ID NO: 13 - protein, 485 aa, Artificial Sequence [sequence omitted]

SEQ ID NO: 14 - protein, 424 aa, Artificial Sequence [sequence omitted]

SEQ ID NO: 15 - protein, 424 aa, Artificial Sequence
  synthetic sequence of GenBank - ADK24690.1
  misc_feature (137)..(137): Xaa can be any naturally occurring amino acid
  misc_feature (140)..(140): Xaa can be any naturally occurring amino acid
MALPTPSDSTLPAEARGRGRRRRLVWTPSQSEALRACFERNPYPGIATRERLAQAIGIPEPRVQIWFQNERSRQLRQHRRESRPWPGRRGPPEGRRKRTAVTGSQTALLLRAFEKDRFPGIAAREELARETGLPESXIQXWFQNRRARHPGQGGRAPAQAGGLCSAAPGGGHPAPSWVAFAHTGAWGTGLPAPHVPCAPGALPQGAFVSQAARAAPALQPSQAAPAEGVSQPAPARGDFAYAAPAPPDGALSHPQAPRWPPHPGKSREDRDPQRDGLPGPCAVAQPGPAQAGPQGQGVLAPPTSQGSPWWGWGRGPQVAGAAWEPQAGAAPPPQPAPPDASASARQGQMQGIPAPSQALQEPAPWSALPCGLLLDELLASPEFLQQAQPLLETEAPGELEASEEAASLEAPLSEEEYRALLEEL

SEQ ID NO: 16 - protein, 424 aa, Artificial Sequence
  synthetic sequence of GenBank - ADK24689.1
  misc_feature (140)..(140): Xaa can be any naturally occurring amino acid
MALPTPSDSTLPAEARGRGRRRRLVWTPSQSEALRACFERNPYPGIATRERLAQAIGIPEPRVQIWFQNERSRQLRQHRRESRPWPGRRGPPEGRRKRTAVTGSQTALLLRAFEKDRFPGIAAREELARETGLPESRIQXWFQNRRARHPGQGGRAPAQAGGLCSAAPGGGHPAPSWVAFAHTGAWGTGLPAPHVPCAPGALPQGAFVSQAARAAPALQPTQAAPAEGVSQPAPARGDFAYAAPAPPDGALSHPQAPRWPPHPGKSREDRDPQRDGLPGPCAVAQPGPAQAGPQGQGVLAPPTSQGSPWWGWGRGPQVAGAAWEPQAGAAPPPQPAPPDASASARQGQMQGIPAPSQALQEPAPWSALPCGLLLDELLASLEFLQQAQPLLETEAPGELEASEEAASLEAPLSEEEYRALLEEL

SEQ ID NO: 17 - protein, 424 aa, Artificial Sequence [sequence omitted]

SEQ ID NO: 18 - protein, 424 aa, Artificial Sequence
  synthetic sequence of GenBank - ADK24693.1
  misc_feature (137)..(137): Xaa can be any naturally occurring amino acid
MALPTPSDSTFPAEARGRGRRRRLVWTPSQSEALRACFERNPYPGIATRERLAQAIGIPEPRVQIWFQNERSRQLRQHRRESRPWPGRRGPPEGRRKRTAVTGSQTALLLRAFEKDRFPGIAAREELARETGLPESXIQIWFQNRRARHPGQGGRAPAQAGGLCSAAPGGGHPAPSWVAFAHTGEWGTGLPAPHVPCAPGALPQGAFVSQAARAAPALQPSQAATAEGVSQPAPARGDFAYAAPAPPDGALSHPQAPRWPPHPGKSREDRDPQRDGLPGPCAVAQPGPAQAGPQGQGVLAPPTSQGSPWWGWGRGPQVAGAAWEPQAGAAPPPQPAPPDASASARQGQMQGIPAPSQALQEPAPWSALPCGLLLDELLASPEFLQQAQPLLETEAPGELEASEEAASLEAPLSEEEYRALLEEL

SEQ ID NO: 19 - protein, 422 aa, Artificial Sequence
  synthetic sequence of GenBank - ADK24712.1
MALPTPSDSTLPAEARGRGRRRRLVWTPSQSEALRACFERNPYPGIATRERLAQAIGIPEPRVQIWFQNERSRQLRQHRRESRPWPGRRGPPEGRRKRTAVTGSQTALLLRAFEKDRFPGIAAREELARETGLPESRIQIWFQNRRARHPGQGGRAPAQAGGLCSAAPGGGHPAPSWVAFAHTGAWGTGLPAPHVPCAPGALPQGAFVSQAARAAPALQPSQAAPAEGISQPAPARGDFAYAAPAPPDGALSHPQAPRWPPHPGKSREDRDPQRDGLPGPCAVAQPGPAQAGPQGQGVLAPPTSQGSPWWGWGRGPQVAGAAWEPQAGAAPPPQPAPPDASARQGQMQGIPAPSQALQEPAPWSALPCGLLLDELLASPEFLQQAQPLLETEAPGELEASEEAASLEAPLSEEEYRALLEEL

SEQ ID NO: 20 - protein, 424 aa, Artificial Sequence
  synthetic sequence of GenBank -  ADK24691.1
  misc_feature (137)..(137): Xaa can be any naturally occurring amino acid
  misc_feature (140)..(140): Xaa can be any naturally occurring amino acid
MALPTPSDSTLPAEARGRGRRRRLVWTPSQSEALRACFERNPYPGIATRERLAQAIGIPEPRVQIWFQNERSRQLRQHRRESRPWPGRRGPPEGRRKRTAVTGSQTALLLRAFEKDRFPGIAAREELARETGLPESXIQXWFQNRRARHPGQGGRAPAQAGGLCSAAPGGGHPAPSWVAFAHTGAWGTGLPAPHVPCAPGALPQGAFVSQAARAAPALQPSQAAPAEGVSQPAPARGDFAYAAPAPPDGALSHPQAPRWPPHPGKSREDRDPQRDGLPGPCAVAQPGPAQAGPQGQGVLAPPTSQGSPWWGWGRGPQVAGAAWEPQAGAAPPPQPAPPDASASARQGQMQGIPAPSQALQEPAPWSALPCGLLLDELLASLEFLQQAQPLLETEAPGELEASEEAASLEAPLSEEEYRALLEEL

SEQ ID NO: 21 - protein, 422 aa, Artificial Sequence
  synthetic sequence of UniProtKB_Swiss-Prot - P0CJ87.1
MALPTPSDSTLPAEARGRGRRRRLVWTPSQSEALRACFERNPYPGIATRERLAQAIGIPEPRVQIWFQNERSRQLRQHRRESRPWPGRRGPPEGRRKRTAVTGSQTALLLRAFEKDRFPGIAAREELARETGLPESRIQIWFQNRRARHPGQGGRAPAQAGGLCSAAPGGGHPAPSWVAFAHTGAWGTGLPAPHVPCAPGALPQGAFVSQAARAAPALQPSQAAPAEGVSQPAPARGDFAYAAPAPPDGALSHPQAPRWPPHPGKSREDRDPQRDGLPGPCAVAQPGPAQAGPQGQGVLAPPTSQGSPWWGWGRGPQVAGAAWEPQAGEAPPPQPAPPDASARQGQMQGIPAPSQALQEPAPWSALPCGLLLDELLASPEFLQQAQPLLETEAPGELEASEEAASLEAPLSEEEYRALLEEL

SEQ ID NO: 22 - protein, 422 aa, Artificial Sequence [sequence omitted]

SEQ ID NO: 23 - protein, 422 aa, Artificial Sequence [sequence omitted]

SEQ ID NO: 24 - protein, 356 aa, Artificial Sequence
  synthetic sequence of GenBank - ADK24695.1
NERSRQLRQHRRESRPWPGRRGPPEGRRKRTAVTGSQTALLLRAFEKDRFPGIAAREELARETGLPESRIQIWFQNRRARHPGQGGRAPAQAGGLCSAAPGGGHPAPSWVAFAHTGAWGTGLPAPHVPCAPGALPQGAFVSQAARAAPALQPSQAAPAEGISQPAPARGDFAYAAPAPPDGALSHPQAPRWPPHPGKSREDRDPQRDGLPGPCAVAQPGPAQAGPQGQGVLAPPTSQGSPWWGWGRGPQVAGAAWEPQAGAAPPPQPAPPDASASARQGQMQGIPAPSQALQEPAPWSALPCGLLLDELLASPEFLQQAQPLLETEAPGELEASEEAASLEAPLSEEEYRALLEEL

SEQ ID NO: 25 - protein, 356 aa, Artificial Sequence
  synthetic sequence of GenBank - ADK24699.1
  misc_feature (293)..(293): Xaa can be any naturally occurring amino acid
  misc_feature (300)..(300): Xaa can be any naturally occurring amino acid
NERSRQLRQHRRESRPWPGRRGPPEGRRKRTAVTGSQTALLLRAFEKDRFPGIAAREELARETGLPESRIQIWFQNRRARHPGQGGRAPAQAGGLCSAAPGGGHPAPSWVAFAHTGAWGTGLPAPHVPCAPGALPQGAFVSQAARAAPALQPSQAAPAEGISQPAPARGDFAYAAPAPPDGALSHPQAPRWPPHPGKSREDRDPQRDGLPGPCAVAQPGPAQAGPQGQGVLAPPTSQGSPWWGWGRGPQVAGAAWEPQAGAAPPPQPAPPDASASARQGQMQGIPAPSQALQXPAPWSAXPCGLLLDELLASPEFLQQAQPLLETEAPGELEASEEAASLEAPLSEEEYRALLEEL

SEQ ID NO: 26 - protein, 323 aa, Artificial Sequence
  synthetic sequence of NCBI Reference Sequence - NP_001768.1 for CD47 isoform 1 precursor
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 27 - protein, 305 aa, Artificial Sequence
  synthetic sequence of NCBI Reference Sequence - NP_942088.1 for CD47 isoform 2 precursor
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRNN

SEQ ID NO: 28 - protein, 80 aa, Artificial Sequence
  synthetic sequence of NCBI Reference Sequence - NP_001278666.1 for signal transducer CD24 isoform a preproprotein
MGRAMVARLGLGLLLLALLLPTQIYSSETTTGTSSNSSQSTSNSGLAPNPTNATTKAAGGALQSTASLFVVSLSLLHLYS

SEQ ID NO: 29 - protein, 80 aa, Artificial Sequence; identical to SEQ ID NO: 28
  synthetic sequence of NCBI Reference Sequence - NP_001278667.1 for signal transducer CD24 isoform a preproprotein

SEQ ID NO: 30 - protein, 129 aa, Artificial Sequence
  synthetic sequence of NCBI Reference Sequence - NP_001278668.1 for signal transducer CD24 isoform b
MVGRFCPESPPGFVRVAATSAVSLDPPSGEPRPGCGYPGPRSAASRVYGCTAPARETGGWAWETLAGAGAKKIYSSETTTGTSSNSSQSTSNSGLAPNPTNATTKAAGGALQSTASLFVVSLSLLHLYS

SEQ ID NO: 31 - protein, 80 aa, Artificial Sequence; identical to SEQ ID NO: 28
  synthetic sequence of NCBI Reference Sequence - NP_037362.1 for signal transducer CD24 isoform a preproprotein

SEQ ID NO: 32 - protein, 305 aa, Artificial Sequence
  synthetic sequence of Mature CD47 isoform 1
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 33 - protein, 287 aa, Artificial Sequence
  synthetic sequence of Mature CD47 isoform 2
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRNN

(19) United States
(12) Patent Application Publication
(10) Pub. No.: US 2023/0025289 A1
(43) Pub. Date: Jan. 26, 2023

(54) CD24 EXPRESSING CELLS AND USES THEREOF

(71) Applicant: Sana Biotechnology, Inc., Seattle, WA (US)

(72) Inventors: Sonja SCHREPFER, San Mateo, CA (US); Steve HARR, Seattle, WA (US)

(21) Appl. No.: 17/637,789

(22) PCT Filed: Aug. 24, 2020
(86) PCT No.: PCT/US20/47639

§ 371 Date: Feb. 23, 2022

Related U.S. Application Data
(60) Provisional application No. 62/891,180, filed on Aug. 23, 2019.

(51) Int. Cl. A61K 35/545; A61K 38/17; A61P 37/06

(52) U.S. Cl. CPC A61K 35/545; A61K 38/177; A61P 37/06

(57) ABSTRACT

Disclosed herein are cells including cells expressing CD24 and related methods of their use and generation. In some embodiments, the cells disclosed herein do not express one or more MHC I and/or MHC II human leukocyte antigens. In some embodiments, the cells are hypoimmunogenic.

### CROSS-REFERENCE TO RELATED APPLICATIONS

This application claims priority to U.S. Provisional Application No. 62/891,180 filed Aug. 23, 2019, the disclosure of which is herein incorporated in its entirety.

### BACKGROUND OF THE INVENTION

Cancers and degenerative diseases pose a disproportionate threat to human health. Often age-related, these diseases result in the progressive deterioration of affected tissues and organs and, ultimately, disability and death of the affected subject. The promise of regenerative medicine is to replace diseased or missing cells with new healthy cells. Over the past five years, a new paradigm for regenerative medicine has emerged—the use of human pluripotent stem cells (hPSCs) to generate any adult cell type for transplantation into patients. In principle, hPSC-based cell therapies have the potential to treat most if not all degenerative illnesses, however the success of such therapies may be limited by a subject's immune response.

Strategies that have been considered to overcome the immune rejection include HLA-matching (e.g. identical twin or umbilical cord banking), the administration of immunosuppressive drugs to the subject, blocking antibodies, bone marrow suppression/mixed chimerism, HLA-matched stem cell respositories, and autologous stem cell therapy.

There remains a need for novel approaches, compositions and methods for overcoming immune rejection associated with cell replacement therapies.

### BRIEF SUMMARY OF THE INVENTION

In one aspect, provided herein is an isolated cell comprising reduced expression of MHC class I and/or MHC class II human leukocyte antigens and a modification to increase expression of CD24 in the cell. In some embodiments, the cell comprises reduced expression of MHC class I and MHC class II human leukocyte antigens.

In some embodiments, the cell further comprises a genetic modification targeting a CIITA gene by a rare-cutting endonuclease that selectively inactivates the CIITA gene. In some embodiments, the cell further comprises a modification to increase expression of one selected from the group consisting of CD47, DUX4, CD27, CD35, CD46, CD55, CD59, CD200, HLA-C, HLA-E, HLA-E heavy chain, HLA-G, PD-L1, IDO1, CTLA4, C1-Inhibitor, IL-10, IL-35, FASL, CCL21, Mfge8, and Serpinb9 in the cell. In some embodiments, the cell further comprises a modification to increase expression of CD47 in the cell. In some embodiments, the cell further comprises a genetic modification targeting a B2M gene by a rare-cutting endonuclease that selectively inactivates the B2M gene. In some embodiments, the cell further comprises a genetic modification targeting an NLRC5 gene by a rare-cutting endonuclease that selectively inactivates the NLRC5 gene.

In some embodiments, the rare-cutting endonuclease is selected from the group consisting of a Cas protein, a TALE-nuclease, a zinc finger nuclease, a meganuclease, and a homing nuclease. In some embodiments, the genetic modification targeting the CIITA gene by the rare-cutting endonuclease comprises a Cas protein or a polynucleotide encoding a Cas protein, and at least one guide ribonucleic acid sequence for specifically targeting the CIITA gene. In some embodiments, the genetic modification targeting the B2M gene by the rare-cutting endonuclease comprises a Cas protein or a polynucleotide encoding a Cas protein, and at least one guide ribonucleic acid sequence for specifically targeting the B2M gene. In some embodiments, the genetic modification targeting the NLRC5 gene by the rare-cutting endonuclease comprises a Cas protein or a polynucleotide encoding a Cas protein, and at least one guide ribonucleic acid sequence for specifically targeting the NLRC5 gene.

In some embodiments, the modification to increase expression of CD24 comprises introducing an expression vector comprising a polynucleotide sequence encoding CD24 into the cell. In some embodiments, the polynucleotide sequence encoding CD24 is a nucleotide sequence encoding a polypeptide sequence having at least 95% sequence identity to a sequence selected from the group consisting of SEQ ID NOS:28-31. In some embodiments, the polynucleotide sequence encoding CD24 is a nucleotide sequence encoding a polypeptide having a sequence selected from the group consisting of SEQ ID NOS:28-31.

In some embodiments, the modification to increase expression of one or more selected from the group consisting of CD47, DUX4, HLA-C, HLA-E, HLA-G, PD-L1, CTLA4, C1-inhibitor, CD46, CD55, CD59, and IL-35 comprises introducing an expression vector comprising a polynucleotide sequence encoding the one or more selected from the group consisting of CD47, DUX4, HLA-C, HLA-E, HLA-G, PD-L1, CTLA4, C1-inhibitor, CD46, CD55, CD59, and IL-35 into the cell. In some embodiments, the modification to increase expression of CD47 comprises introducing an expression vector comprising a polynucleotide sequence encoding CD47 into the cell.

In some embodiments, the expression vector for increasing expression of any of the polypeptides described is an inducible expression vector. In some embodiments, the expression vector is a viral vector.

In some embodiments, the modification to increase expression of CD24 comprises introducing a polynucleotide sequence encoding CD24 into a selected locus of the cell. In some embodiments, the polynucleotide sequence encoding CD24 is a nucleotide sequence encoding a polypeptide sequence having at least 95% sequence identity to a sequence selected from the group consisting of SEQ ID NOS:28-31. In some embodiments, the polynucleotide sequence encoding CD24 is a nucleotide sequence encoding a polypeptide having a sequence selected from the group consisting of SEQ ID NOS:28-31.

In some embodiments, the modification to increase expression of a polypeptide selected from the group consisting of CD47, CD35, DUX4, HLA-C, HLA-E, HLA-G, PD-L1, CTLA4, C1-inhibitor, CD46, CD55, CD59, and IL-35 comprises introducing a polynucleotide sequence encoding the polypeptide selected from the group consisting of CD47, CD35, DUX4, HLA-C, HLA-E, HLA-G, PD-L1, CTLA4, C1-inhibitor, CD46, CD55, CD59, and IL-35 into a selected locus of the cell. In some embodiments, the modification to increase expression of CD47 comprises introducing a polynucleotide sequence encoding CD47 into a selected locus of the cell. In some embodiments, the selected locus for the polynucleotide sequence encoding CD24 and/or the selected locus for the polynucleotide sequence encoding the polypeptide selected from the group consisting of CD47, CD35, DUX4, HLA-C, HLA-E, HLA-G, PD-L1, CTLA4, C1-inhibitor, CD46, CD55, CD59, and IL-35 is a safe harbor locus. In some embodiments, the safe harbor is selected from the group consisting of an AAVS1 locus, CCR5 locus, CLYBL locus, ROSA26 locus, and SHS231 locus.

In some embodiments, the cell further comprises an inducible suicide switch.

In some embodiments, the cell described above is selected from the group consisting of a stem cell, a differentiated cell, a pluripotent stem cell, an induced pluripotent stem cell, an adult stem cell, a progenitor cell, a somatic cell, a primary T cell and a chimeric antigen receptor T cell.

In some aspects, provided herein is a method of preparing a cell comprising CD24 (e.g., a CD24 polypeptide), the method comprises introducing an expression vector comprising a polynucleotide sequence encoding CD24 into the cell, thereby producing the cell comprising CD24. In some embodiments, the polynucleotide sequence encoding CD24 is a nucleotide sequence encoding a polypeptide sequence having at least 95% sequence identity to a sequence selected from the group consisting of SEQ ID NOS:28-31. In some embodiments, the polynucleotide sequence encoding CD24 is a nucleotide sequence encoding a polypeptide having a sequence selected from the group consisting of SEQ ID NOS:28-31.

In some embodiments, the cell comprising CD24 further comprises a genetic modification targeting a CIITA gene comprising a rare-cutting endonuclease selected from a group consisting of a Cas protein, a TALE-nuclease, a zinc finger nuclease, a meganuclease, and a homing nuclease for targeting the CIITA gene. In some embodiments, the genetic modification comprises a Cas protein or a polynucleotide encoding a Cas protein, and at least one guide ribonucleic acid for specifically targeting the CIITA gene.

In some embodiments, the expression vector comprising the polynucleotide sequence encoding CD24 is an inducible expression vector. In some embodiments, the expression vector is a viral vector.

In some embodiments, the cell comprising CD24 further comprises a second expression vector comprising a polynucleotide sequence encoding one selected from the group consisting of CD47, CD35, DUX4, HLA-C, HLA-E, HLA-G, PD-L1, CTLA4, C1-inhibitor, CD46, CD55, CD59, and IL-35. In some embodiments, the second expression vector comprises a polynucleotide sequence encoding CD47. In some embodiments, the second expression vector is an inducible expression vector. In some embodiments, the second expression vector is a viral vector.

In some embodiments, the cell comprising CD24 further comprises a genetic modification targeting a B2M gene comprising a rare-cutting endonuclease selected from a group consisting of a Cas protein, a TALE-nuclease, a zinc finger nuclease, a meganuclease, and a homing nuclease for specifically targeting the B2M gene. In some instances, the genetic modification comprises a Cas protein or a polynucleotide encoding a Cas protein, and at least one guide ribonucleic acid for specifically targeting the B2M gene.

In some embodiments, the cell comprising CD24 further comprises a genetic modification targeting an NLRC5 gene comprising a rare-cutting endonuclease selected from a group consisting of a Cas protein, a TALE-nuclease, a zinc finger nuclease, a meganuclease, and a homing nuclease for specifically targeting the NLRC5 gene. In some instances, the genetic modification comprises a Cas protein or a polynucleotide encoding a Cas protein, and at least one guide ribonucleic acid for specifically targeting the NLRC5 gene.

In some embodiments, the cell described above is selected from the group consisting of a stem cell, a differentiated cell, an embryonic stem cell, a pluripotent stem cell, an induced pluripotent stem cell, a hematopoietic stem cell, an adult stem cell, a progenitor cell, a somatic cell, a primary T cell and a chimeric antigen receptor T cell.

Provided herein is a method of preparing a hypoimmunogenic stem cell comprising introducing a polynucleotide sequence encoding CD24 into a selected locus of the stem cell, thereby producing a hypoimmunogenic stem cell.

In some embodiments, the polynucleotide sequence encoding CD24 is a nucleotide sequence encoding a polypeptide sequence having at least 90% or at least 95% sequence identity to a sequence selected from the group consisting of SEQ ID NOS:28-31. In some embodiments, the polynucleotide sequence encoding CD24 is a nucleotide sequence encoding a polypeptide having a sequence selected from the group consisting of SEQ ID NOS:28-31.

In some embodiments, the method further comprises generating a genetic modification targeting a CIITA gene in a stem cell comprising introducing a rare-cutting endonuclease that selectively inactivates the CIITA gene into the stem cell, wherein the rare-cutting endonuclease is selected from a group consisting of a Cas protein, a TALE-nuclease, a zinc finger nuclease, a meganuclease, and a homing nuclease. In some embodiments, the introducing of the rare-cutting endonuclease comprises introducing a Cas protein or a polynucleotide encoding a Cas protein, and at least one guide ribonucleic acid for specifically targeting the CIITA gene.

In some embodiments, the selected locus for the polynucleotide sequence encoding CD24 is a safe harbor locus. In some embodiments, the safe harbor locus for the polynucleotide sequence encoding CD24 is selected from the group consisting of an AAVS1 locus, CCR5 locus, CLYBL locus, ROSA26 locus, and SHS231 locus.

In some embodiments, the method of preparing a hypoimmunogenic stem cell further comprises introducing a polynucleotide sequence encoding a polypeptide selected from the group consisting of CD47, CD35, DUX4, HLA-C, HLA-E, HLA-G, PD-L1, CTLA4, C1-inhibitor, CD46, CD55, CD59, and IL-35 into a selected locus of the stem cell. In certain embodiments, the method further comprises introducing a polynucleotide sequence encoding CD47 into a selected locus of the stem cell. In some embodiments, the selected locus is a safe harbor locus. In some embodiments, the safe harbor locus is selected from the group consisting of an AAVS1 locus, CCR5 locus, CLYBL locus, ROSA26 locus, and SHS231 locus.

In some embodiments, the method of preparing a hypoimmunogenic stem cell further comprises generating a genetic modification targeting a B2M gene in a stem cell comprising introducing a rare-cutting endonuclease that selectively inactivates the B2M gene into the stem cell, wherein the rare-cutting endonuclease is selected from a group consisting of a Cas protein, a TALE-nuclease, a zinc finger nuclease, a meganuclease, and a homing nuclease. In some embodiments, the introducing of the rare-cutting endonuclease comprises introducing a Cas protein or a polynucleotide encoding a Cas protein, and at least one guide ribonucleic acid for specifically targeting the B2M gene.

In some embodiments, the method of preparing a hypoimmunogenic stem cell further comprises generating a genetic modification targeting an NLRC5 gene in a stem cell comprising introducing a rare-cutting endonuclease that selectively inactivates the NLRC5 gene into the stem cell, wherein the rare-cutting endonuclease is selected from a group consisting of a Cas protein, a TALE-nuclease, a zinc finger nuclease, a meganuclease, and a homing nuclease. In some embodiments, the introducing of the rare-cutting endonuclease comprises introducing a Cas protein or a polynucleotide encoding a Cas protein, and at least one guide ribonucleic acid for specifically targeting the NLRC5 gene.

In some embodiments, the method of preparing a hypoimmunogenic stem cell further comprises introducing an expression vector comprising an inducible suicide switch into the stem cell.

In some aspects, provided is a method of preparing a differentiated hypoimmunogenic cell comprising culturing under differentiation conditions a hypoimmunogenic stem cell prepared according to any method disclosed herein, thereby preparing a differentiated hypoimmunogenic cell.

In some embodiments, the differentiation conditions are appropriate for differentiation of a stem cell into a cell type selected from the group consisting of a cardiac cell, neural cell, endothelial cell, T cell, pancreatic islet cell, retinal pigmented epithelium cell, kidney cell, liver cell, thyroid cell, skin cell, blood cell, and epithelial cell.

In some aspects, provided is a method of treating a patient in need of cell therapy comprising administering a population of differentiated hypoimmunogenic cells prepared according to a method disclosed herein.

Provided herein is a cell that expresses CD24, and has reduced expression of MHC class I human leukocyte antigens.

Provided herein is a cell that expresses CD24, and has reduced expression of MHC class I and/or MHC class II human leukocyte antigens.

Provided herein is a cell that does not express CIITA, expresses CD24, and has reduced expression of MHC class I and/or MHC class II human leukocyte antigens.

Provided herein is a cell that does not express B2M, expresses CD24, and has reduced expression of MHC class I and/or MHC class II human leukocyte antigens.

Provided herein is a cell that does not express NLRC5, expresses CD24, and has reduced expression of MHC class I and/or MHC class II human leukocyte antigens.

Provided herein is a cell that expresses CD24 and at least one selected from the group consisting of CD47, DUX4, HLA-C, HLA-E, HLA-G, PD-L1, CTLA4, C1-inhibitor, CD46, CD55, CD59, and IL-35, and has reduced expression of MHC class I and/or MHC class II human leukocyte antigens.

Provided herein is a cell that expresses CD24 and CD47, and has reduced expression of MHC class I and/or MHC class II human leukocyte antigens.

Provided herein is a cell that does not express CIITA, expresses CD24 and at least one polypeptide selected from the group consisting of CD47, DUX4, HLA-C, HLA-E, HLA-G, PD-L1, CTLA4, C1-inhibitor, CD46, CD55, CD59, and IL-35, and has reduced expression of MHC class I and/or MHC class II human leukocyte antigens.

Provided herein is a cell that does not express CIITA, expresses CD24 and CD47, and has reduced expression of MHC class I and/or MHC class II human leukocyte antigens.

Provided herein is a cell that does not express CIITA and B2M, expresses CD24, and has reduced expression of MHC class I and/or MHC class II human leukocyte antigens.

Provided herein is a cell that does not express CIITA and B2M, expresses CD24 and at least one polypeptide selected from the group consisting of CD47, DUX4, HLA-C, HLA-E, HLA-G, PD-L1, CTLA4, C1-inhibitor, CD46, CD55, CD59, and IL-35, and has reduced expression of MHC class I and/or MHC class II human leukocyte antigens.

Provided herein is a cell that does not express CIITA and B2M, expresses CD24 and CD47, and has reduced expression of MHC class I and/or MHC class II human leukocyte antigens.

Provided herein is a cell that does not express CIITA and NLRC5, expresses CD24, and has reduced expression of MHC class I and/or MHC class II human leukocyte antigens.

Provided herein is a cell that does not express CIITA and NLRC5, expresses CD24 and at least one polypeptide selected from the group consisting of CD47, CD35, DUX4, HLA-C, HLA-E, HLA-G, PD-L1, CTLA4, C1-inhibitor, CD46, CD55, CD59, and IL-35, and has reduced expression of MHC class I and/or MHC class II human leukocyte antigens.

Provided herein is a cell that does not express CIITA and NLRC5, expresses CD24 and CD47, and has reduced expression of MHC class I and/or MHC class II human leukocyte antigens.

Provided herein is a cell that does not express CIITA, B2M, and NLRC5, expresses CD24 and at least one polypeptide selected from the group consisting of CD47, CD35, DUX4, HLA-C, HLA-E, HLA-G, PD-L1, CTLA4, C1-inhibitor, CD46, CD55, CD59, and IL-35, and has reduced expression of MHC class I and/or MHC class II human leukocyte antigens.

Provided herein is a cell that does not express CIITA, B2M, and NLRC5, expresses CD24 and CD47, and has reduced expression of MHC class I and/or MHC class II human leukocyte antigens.

In some embodiments, any one of the cell described above is selected from the group consisting of a stem cell, a differentiated cell, a pluripotent stem cell, an induced pluripotent stem cell, an adult stem cell, a progenitor cell, a somatic cell, a primary T cell and a chimeric antigen receptor T cell.

Also, provided herein is a differentiated cell generated from any pluripotent stem cell or induced pluripotent stem cell described herein by culturing under differentiation conditions to generate a differentiated cell selected from the group consisting of a cardiac cell, neural cell, endothelial cell, T cell, pancreatic islet cell, retinal pigmented epithelium (RPE) cell, kidney cell, liver cell, thyroid cell, skin cell, blood cell, and epithelial cell.

In one aspect of the disclosure, provided herein is an isolated stem cell comprising an exogenous CD24 polypeptide. In some embodiments, the cell expresses a nucleotide (e.g., a polynucleotide) sequence encoding a CD24 polypeptide having at least 95% (e.g., 95%, 96%, 97%, 98%, 99%, or more) sequence identity to a sequence selected from the group consisting of SEQ ID NO:28, SEQ ID NO:29, SEQ ID NO:30, and SEQ ID NO:31. In some embodiments, the cell expresses a nucleotide sequence encoding a CD24 polypeptide having at least 90% (e.g., 90%, 91%, 92%, 93%, 94%, 95%, 96%, 97%, 98%, 99%, or more) sequence identity to a sequence selected from the group consisting of SEQ ID NO:28, SEQ ID NO:29, SEQ ID NO:30, and SEQ ID NO:31. In some embodiments, the cell expresses a nucleotide sequence encoding a CD24 polypeptide selected from the group consisting of SEQ ID NO:28, SEQ ID NO:29, SEQ ID NO:30, and SEQ ID NO:31.

In some embodiments, the isolated cell has reduced expression of MHC class I human leukocyte antigens. In other embodiments, the cell has reduced expression of MHC II human leukocyte antigens. In yet other embodiments, the cell has reduced expression of MHC class I and MHC II human leukocyte antigens. In some embodiments, the cell has reduced expression of CIITA. In certain embodiments, the cell has reduced expression of B2M. In particular embodiments, the cell has reduced expression of NLRC5.

In some embodiments, the isolated cell further comprises a genome modification targeting CIITA to reduce expression of CIITA. In some embodiments, the genome modification comprises a rare-cutting endonuclease. In some embodiments, the rare-cutting endonuclease is selected from the group consisting of a Cas protein, a TALE-nuclease, a zinc finger nuclease, a meganuclease, and a homing endonuclease. In certain embodiments, the rare-cutting endonuclease comprises a Cas protein or a polynucleotide encoding a Cas protein targeting CIITA. In some instances, the cell further comprises at least one guide ribonucleic acid sequence recognized by the Cas protein targeting CIITA. In some embodiments, the at least one guide ribonucleic acid sequence for targeting CIITA is selected from the group consisting of SEQ ID NOS:5184-36352 of WO2016183041, the disclosure including sequence listing is incorporated by reference in its entirety.

In other embodiments, the isolated cell further comprises a genome modification targeting B2M to reduce expression of B2M. In some embodiments, the genome modification comprises a rare-cutting endonuclease. In some embodiments, the rare-cutting endonuclease is selected from the group consisting of a Cas protein, a TALE-nuclease, a zinc finger nuclease, a meganuclease, and a homing endonuclease. In some embodiments, the rare-cutting endonuclease comprises a Cas protein or a polynucleotide encoding a Cas protein targeting B2M. In some instances, the cell further comprises at least one guide ribonucleic acid sequence recognized by the Cas protein targeting B2M. In some embodiments, the at least one guide ribonucleic acid sequence for targeting B2M is selected from the group consisting of SEQ ID NOS:81240-85644 of WO2016183041, the disclosure including sequence listing is incorporated by reference in its entirety.

In some embodiments, the isolated cell further comprises a genome modification targeting NLRC5 to reduce expression of NLRC5. In some embodiments, the genome modification comprises a rare-cutting endonuclease. In some embodiments, the rare-cutting endonuclease is selected from the group consisting of a Cas protein, a TALE-nuclease, a zinc finger nuclease, a meganuclease, and a homing endonuclease. In some embodiments, the rare-cutting endonuclease comprises a Cas protein or a polynucleotide encoding a Cas protein targeting NLRC5. In some instances, the cell further comprises at least one guide ribonucleic acid sequence recognized by the Cas protein targeting NLRC5. In some embodiments, the at least one guide ribonucleic acid sequence targeting NLRC5 is selected from the group consisting of SEQ ID NOS:36353-81239 of WO2016183041, the disclosure including sequence listing is incorporated by reference in its entirety.

In some embodiments, the isolated cell further comprises a gene expression modification to reduce expression of CIITA. In certain embodiments, the cell further comprises a gene expression modification to reduce expression of B2M. In other embodiments, the cell further comprises a gene expression modification to reduce expression of NLRC5. In some embodiments, the gene expression modification comprises one selected from the group consisting of an siRNA, shRNA, microRNA, antisense RNA, and another RNA-mediated inhibition molecule.

In some embodiments, the isolated cell further comprises an exogenous immunoregulatory factor selected from the group consisting of HLA-C, HLA-E, HLA-G, PD-L1, CTLA-4-Ig, C1-inhibitor, and IL-35. In other embodiments, the cell further comprises one or more exogenous immunoregulatory factors selected from the group consisting of HLA-C, HLA-E, HLA-G, PD-L1, CTLA-4-Ig, C1-inhibitor, and IL-35.

In some embodiments, the isolated cell outlined herein is selected from the group consisting of a stem cell, an embryonic stem cell, a pluripotent stem cell, and an adult stem cell.

In some embodiments, provided herein is an isolated cell generated from any stem cell described herein under differentiation conditions.

In some embodiments, provided herein is an isolated cell that is hypoimmunogenic, e.g., hypoimmunogenic to a patient upon administration.

In one aspect, provided herein is a method of preparing a stem cell comprising an exogenous CD24 polypeptide comprising introducing an expression vector comprising a nucleotide sequence encoding a CD24 polypeptide having at least 95% (e.g., 95%, 96%, 97%, 98%, 99%, or more) sequence identity to a sequence selected from the group consisting of SEQ ID NO:28, SEQ ID NO:29, SEQ ID NO:30, and SEQ ID NO:31. In some embodiments, the CD24 polypeptide sequence is selected from the group consisting of SEQ ID NO:28, SEQ ID NO:29, SEQ ID NO:30, and SEQ ID NO:31. In another aspect, provided herein is a method of preparing a stem cell comprising an exogenous CD24 polypeptide comprising introducing an expression vector comprising a nucleotide sequence encoding a CD24 polypeptide having at least 90% (e.g., 90%, 91%, 92%, 93%, 94%, 95%, 96%, 97%, 98%, 99%, or more) sequence identity to a sequence selected from the group consisting of SEQ ID NO:28, SEQ ID NO:29, SEQ ID NO:30, and SEQ ID NO:31. In some embodiments, the CD24 polypeptide sequence is selected from the group consisting of SEQ ID NO:28, SEQ ID NO:29, SEQ ID NO:30, and SEQ ID NO:31.

In some embodiments, the expression vector of the exogenous CD24 polypeptide is an inducible expression vector. In some embodiments, the expression vector is a viral vector. In certain embodiments, the expression vector specifically targets a safe harbor locus. In particular embodiments, the safe harbor locus is an AAVS1 locus.

In some embodiments, the method of preparing the stem cell further comprises introducing into the cell a rare-cutting endonuclease that selectively inactivates the CIITA gene. In some embodiments, the rare-cutting endonuclease is selected from the group consisting of a Cas protein, a TALE-nuclease, a zinc finger nuclease, a meganuclease, and a homing endonuclease. In some instance, the method also includes introducing at least one guide ribonucleic acid sequence for specifically targeting the CIITA gene, wherein the rare-cutting endonuclease comprises a Cas protein or a polynucleotide encoding a Cas protein. In some embodiments, the at least one guide ribonucleic acid sequence for specifically targeting the CIITA gene is selected from the group consisting of SEQ ID NOS:5184-36352 of WO2016183041, the disclosure including sequence listing is incorporated by reference in its entirety.

In some embodiments, the method further comprises introducing into the cell a rare-cutting endonuclease that selectively inactivates the B2M gene. In some embodiments, the rare-cutting endonuclease is selected from the group consisting of a Cas protein, a TALE-nuclease, a zinc finger nuclease, a meganuclease, and a homing endonuclease. In some instance, the method also includes introducing at least one guide ribonucleic acid sequence for specifically targeting the B2M gene, wherein the rare-cutting endonuclease comprises a Cas protein or a polynucleotide encoding a Cas protein. In some embodiments, the at least one guide ribonucleic acid sequence for specifically targeting the B2M gene is selected from the group consisting of SEQ ID NOS:81240-85644 of WO2016183041, the disclosure including sequence listing is incorporated by reference in its entirety.

In some embodiments, the method further comprises introducing into the cell a rare-cutting endonuclease that selectively inactivates the NLRC5 gene. In some embodiments, the rare-cutting endonuclease is selected from the group consisting of a Cas protein, a TALE-nuclease, a zinc finger nuclease, a meganuclease, and a homing endonuclease. In some instance, the method also includes introducing at least one guide ribonucleic acid sequence for specifically targeting the NLRC5 gene, wherein the rare-cutting endonuclease comprises a Cas protein or a polynucleotide encoding a Cas protein. In some embodiments, the at least one guide ribonucleic acid sequence for specifically targeting the NLRC5 gene is selected from the group consisting of SEQ ID NOS:36353-81239 of WO2016183041, the disclosure including sequence listing is incorporated by reference in its entirety.

In some embodiments, the method further comprises introducing into the cell a gene expression modification molecule to reduce expression of CIITA, wherein the gene expression modification molecule comprises one selected from the group consisting of siRNA, shRNA, microRNA, antisense RNA, and another RNA-mediated inhibition molecule that specifically targets CIITA. In some embodiments, the method further comprises introducing into the cell a gene expression modification molecule to reduce expression of B2M, wherein the gene expression modification molecule comprises one selected from the group consisting of siRNA, shRNA, microRNA, antisense RNA, and another RNA-mediated inhibition molecule that specifically targets B2M. In some embodiments, the method further comprises introducing into the cell a gene expression modification molecule to reduce expression of NLRC5, wherein the gene expression modification molecule comprises one selected from the group consisting of siRNA, shRNA, microRNA, antisense RNA, and another RNA-mediated inhibition molecule that specifically targets NLRC5.

In some embodiments, the method further comprises introducing an expression vector comprising a nucleotide sequence encoding a tolerogenic polypeptide selected from the group consisting of HLA-C, HLA-E, HLA-G, PD-L1, CTLA-4-Ig, C1-inhibitor, and IL-35.

In some embodiments, the method further comprises introducing at least two expression vectors, wherein the first expression vector comprises a first nucleotide sequence encoding a first tolerogenic polypeptide selected from the group consisting of HLA-C, HLA-E, HLA-G, PD-L1, CTLA-4-Ig, C1-inhibitor, and IL-35, and the second expression vector comprises a second nucleotide sequence encoding a different tolerogenic polypeptide selected from the group consisting of HLA-C, HLA-E, HLA-G, PD-L1, CTLA-4-Ig, C1-inhibitor, and IL-35.

In some embodiments, the expression vector, the first expression vector, and/or the second expression vector described herein is an inducible expression vector. In some embodiments, the expression vector, the first expression vector, and/or the second expression vector described herein is a viral vector. In some embodiments, the expression vector, the first expression vector, and/or the second expression vector described herein specifically targets a safe harbor locus. In some instances, the safe harbor locus is a AAVS1 locus.

In some embodiments, the method further comprises introducing an expression vector comprising an inducible suicide switch into the stem cell.

In some embodiments, the stem cell described above is selected from the group consisting of a pluripotent stem cell, an induced pluripotent stem cell, an embryonic stem cell, and an adult stem cell.

In some embodiments, the stem cell has reduced expression of MHC class I human leukocyte antigens compared to an unmodified stem cell. In some embodiments, the stem cell has reduced expression of MHC II human leukocyte antigens compared to an unmodified stem cell. In some embodiments, the stem cell has reduced expression of MHC class I and MHC II human leukocyte antigens compared to an unmodified stem cell.

In another aspect, provided herein is a method of preparing a differentiated cell comprising culturing under differentiation conditions any one of the stem cells described herein or any one of the stem cells prepared according to the method outlined herein, thereby preparing a differentiated cell. In some embodiments, the differentiation conditions are appropriate for differentiation of a stem cell into a cell type selected from the group consisting of cardiac cells, liver cell, kidney cells, pancreatic cells, neural cells, immune cells, mesenchymal cells, and endothelial cells.

In another aspect, provided herein a method of treating a patient in need of cell based therapy such as, but not limited to, cell replacement therapy. The method comprises administering a population of differentiated cells prepared according to any method outlined herein.

Provided herein is a stem cell expressing an exogenous CD24 polypeptide and a reduced expression level of MHC class I human leukocyte antigens. Provided herein is a stem cell expressing an exogenous CD24 polypeptide and a reduced expression level of MHC class II human leukocyte antigens. Provided herein is a stem cell expressing an exogenous CD24 polypeptide and a reduced expression level of MHC class I and class II human leukocyte antigens.

Provided herein is a stem cell expressing an exogenous CD24 polypeptide and a reduced expression level of CIITA. Provided herein is a stem cell expressing an exogenous CD24 polypeptide and a reduced expression level of B2M. Provided herein is a stem cell expressing an exogenous CD24 polypeptide and a reduced expression level of NLRC5. Provided herein is a stem cell expressing an exogenous CD24 polypeptide and reduced expression levels of CIITA, B2M, NLRC5, and a combination thereof.

Provided herein is a stem cell expressing an exogenous CD24 polypeptide and one or more tolerogenic factors selected from the group consisting of HLA-C, HLA-E, HLA-G, PD-L1, CTLA-4-Ig, C1-inhibitor, and IL-35. Provided herein is a stem cell expressing an exogenous CD24 polypeptide, one or more tolerogenic factors selected from the group consisting of HLA-C, HLA-E, HLA-G, PD-L1, CTLA-4-Ig, C1-inhibitor, and IL-35, and a reduced expression level of CIITA. Provided herein is a stem cell expressing an exogenous CD24 polypeptide, one or more tolerogenic factors selected from the group consisting of HLA-C, HLA-E, HLA-G, PD-L1, CTLA-4-Ig, C1-inhibitor, and IL-35, and a reduced expression level of B2M. Provided herein is a stem cell expressing an exogenous CD24 polypeptide, one or more tolerogenic factors selected from the group consisting of HLA-C, HLA-E, HLA-G, PD-L1, CTLA-4-Ig, C1-inhibitor, and IL-35, and a reduced expression level of NLRC5. Provided herein is a stem cell expressing an exogenous CD24 polypeptide, one or more tolerogenic factors selected from the group consisting of HLA-C, HLA-E, HLA-G, PD-L1, CTLA-4-Ig, C1-inhibitor, and IL-35, and reduced expression levels of CIITA, B2M, NLRC5, and a combination thereof.

Provided herein is a differentiated cell generated from a stem cell expressing an exogenous CD24 polypeptide and a reduced expression level of MHC class I human leukocyte antigens. Provided herein is a differentiated cell generated from stem cell expressing an exogenous CD24 polypeptide and a reduced expression level of MHC class II human leukocyte antigens. Provided herein is a differentiated cell generated from a stem cell expressing an exogenous CD24 polypeptide and a reduced expression level of MHC class I and class II human leukocyte antigens.

Provided herein is a differentiated cell generated from a stem cell expressing an exogenous CD24 polypeptide and a reduced expression level of CIITA. Provided herein is a differentiated cell generated from a stem cell expressing an exogenous CD24 polypeptide and a reduced expression level of B2M. Provided herein is a differentiated cell generated from a stem cell expressing an exogenous CD24 polypeptide and a reduced expression level of NLRC5. Provided herein is a differentiated cell generated from a stem cell expressing an exogenous CD24 polypeptide and reduced expression levels of CIITA, B2M, NLRC5, and a combination thereof.

Provided herein is a differentiated cell generated from a stem cell expressing an exogenous CD24 polypeptide and one or more tolerogenic factors selected from the group consisting of HLA-C, HLA-E, HLA-G, PD-L1, CTLA-4-Ig, C1-inhibitor, and IL-35. Provided herein is a differentiated cell generated from a stem cell expressing an exogenous CD24 polypeptide, one or more tolerogenic factors selected from the group consisting of HLA-C, HLA-E, HLA-G, PD-L1, CTLA-4-Ig, C1-inhibitor, and IL-35, and a reduced expression level of CIITA. Provided herein is a differentiated cell generated from a stem cell expressing an exogenous CD24 polypeptide, one or more tolerogenic factors selected from the group consisting of HLA-C, HLA-E, HLA-G, PD-L1, CTLA-4-Ig, C1-inhibitor, and IL-35, and a reduced expression level of B2M. Provided herein is a differentiated cell generated from a stem cell expressing an exogenous CD24 polypeptide, one or more tolerogenic factors selected from the group consisting of HLA-C, HLA-E, HLA-G, PD-L1, CTLA-4-Ig, C1-inhibitor, and IL-35, and a reduced expression level of NLRC5. Provided herein is a differentiated cell generated from a stem cell expressing an exogenous CD24 polypeptide, one or more tolerogenic factors selected from the group consisting of HLA-C, HLA-E, HLA-G, PD-L1, CTLA-4-Ig, C1-inhibitor, and IL-35, and reduced expression levels of CIITA, B2M, NLRC5, and a combination thereof.

In some embodiments, the stem cell of the present invention is hypoimmunogenic, e.g., hypoimmunogenic to a patient upon administration. In some embodiments, the differentiated cell of the present invention is hypoimmunogenic, e.g., hypoimmunogenic to a patient upon administration.

In some embodiments, provided herein is a method of treating a patient in need of cell therapy (in some instances, cell replacement therapy) comprising administering a population of differentiated cells comprising a differentiated cell generated from a stem cell expressing an exogenous CD24 polypeptide and a reduced expression level of MHC class I human leukocyte antigens.

In some embodiments, provided herein is a method of treating a patient in need of cell therapy (in some instances, cell replacement therapy) comprising administering a population of differentiated cells comprising a differentiated cell generated from stem cell expressing an exogenous CD24 polypeptide and a reduced expression level of MHC class II human leukocyte antigens.

In some embodiments, provided herein is a method of treating a patient in need of cell therapy comprising administering a population of differentiated cells comprising a differentiated cell generated from a stem cell expressing an exogenous CD24 polypeptide and a reduced expression level of MHC class I and class II human leukocyte antigens.

In some embodiments, provided herein is a method of treating a patient in need of cell therapy (in some instances, cell replacement therapy) comprising administering a population of differentiated cells comprising a differentiated cell generated from a stem cell expressing an exogenous CD24 polypeptide and a reduced expression level of CIITA. In some embodiments, provided herein is a method of treating a patient in need of cell therapy comprising administering a population of differentiated cells comprising a differentiated cell generated from a stem cell expressing an exogenous CD24 polypeptide and a reduced expression level of B2M. In some embodiments, provided herein is a method of treating a patient in need of cell therapy comprising administering a population of differentiated cells comprising a differentiated cell generated from a stem cell expressing an exogenous CD24 polypeptide and a reduced expression level of NLRC5. In some embodiments, provided herein is a method of treating a patient in need of cell therapy comprising administering a population of differentiated cells comprising a differentiated cell generated from a stem cell expressing an exogenous CD24 polypeptide and reduced expression levels of CIITA, B2M, NLRC5, and a combination thereof.

In some embodiments, provided herein is a method of treating a patient in need of cell therapy (in some instances, cell replacement therapy) comprising administering a population of differentiated cells comprising a differentiated cell generated from a stem cell expressing an exogenous CD24 polypeptide and one or more tolerogenic factors selected from the group consisting of HLA-C, HLA-E, HLA-G, PD-L1, CTLA-4-Ig, C1-inhibitor, and IL-35. In some embodiments, provided herein is a method of treating a patient in need of cell therapy comprising administering a population of differentiated cells comprising a differentiated cell generated from a stem cell expressing an exogenous CD24 polypeptide, one or more tolerogenic factors selected from the group consisting of HLA-C, HLA-E, HLA-G, PD-L1, CTLA-4-Ig, C1-inhibitor, and IL-35, and a reduced expression level of CIITA. In some embodiments, provided herein is a method of treating a patient in need of cell therapy comprising administering a population of differentiated cells comprising a differentiated cell generated from a stem cell expressing an exogenous CD24 polypeptide, one or more tolerogenic factors selected from the group consisting of HLA-C, HLA-E, HLA-G, PD-L1, CTLA-4-Ig, C1-inhibitor, and IL-35, and a reduced expression level of B2M. In some embodiments, provided herein is a method of treating a patient in need of cell therapy comprising administering a population of differentiated cells comprising a differentiated cell generated from a stem cell expressing an exogenous CD24 polypeptide, one or more tolerogenic factors selected from the group consisting of HLA-C, HLA-E, HLA-G, PD-L1, CTLA-4-Ig, C1-inhibitor, and IL-35, and a reduced expression level of NLRC5. In some embodiments, provided herein is a method of treating a patient in need of cell therapy comprising administering a population of differentiated cells comprising a differentiated cell generated from a stem cell expressing an exogenous CD24 polypeptide, one or more tolerogenic factors selected from the group consisting of HLA-C, HLA-E, HLA-G, PD-L1, CTLA-4-Ig, C1-inhibitor, and IL-35, and reduced expression levels of CIITA, B2M, NLRC5, and a combination thereof.

Detailed descriptions of hypoimmunogenic cells, methods of producing thereof, and methods of using thereof are found in WO2016183041 filed May 9, 2015, WO2018132783 filed Jan. 14, 2018, and WO2018175390 filed Mar. 20, 2018, the disclosures including the sequence listings and Figures are incorporated herein by reference in their entirety.

Other objects, advantages and embodiments of the invention will be apparent from the detailed description following.

### BRIEF DESCRIPTION OF THE DRAWINGS

FIGS. 1A-1H depict sequences of a DUX4 polynucleotide and DUX4, CD47, and CD24 polypeptides as depicted in SEQ ID NOS:1-33.

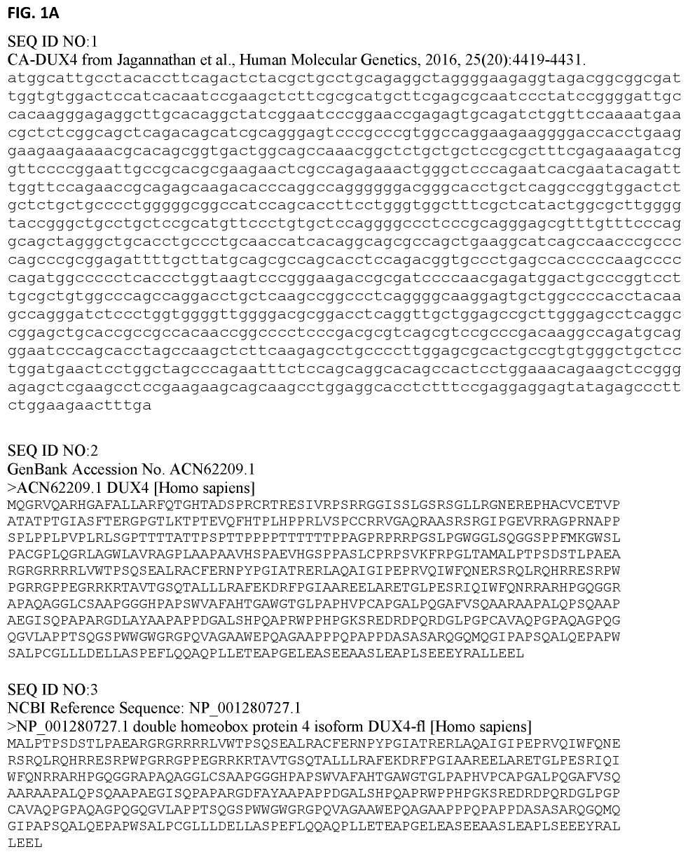

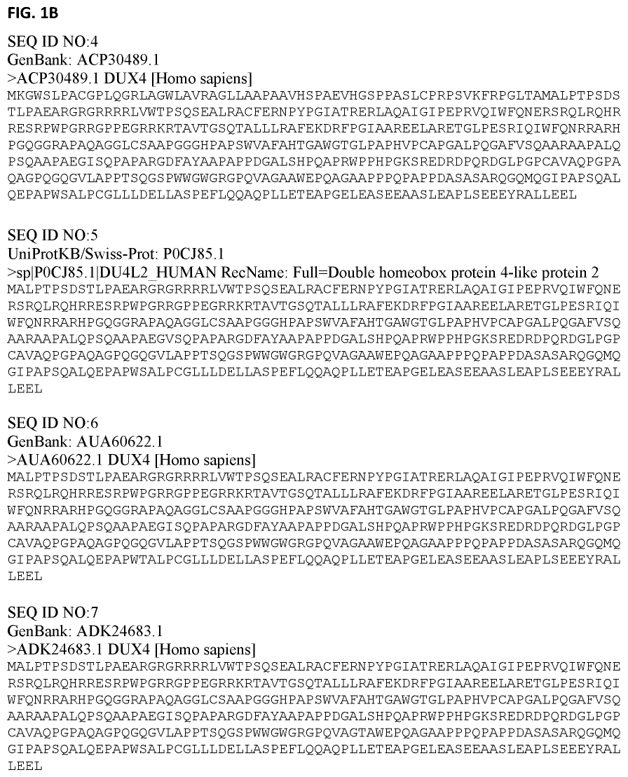

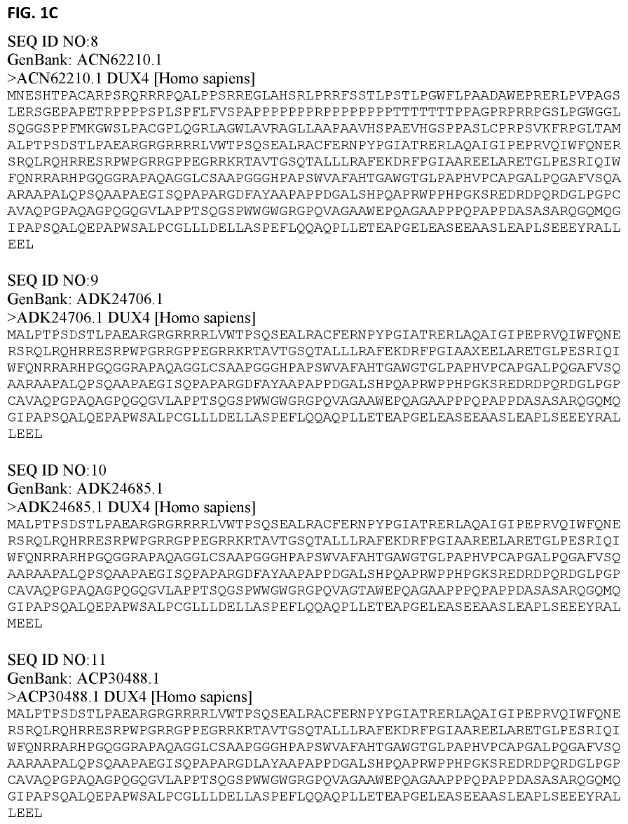

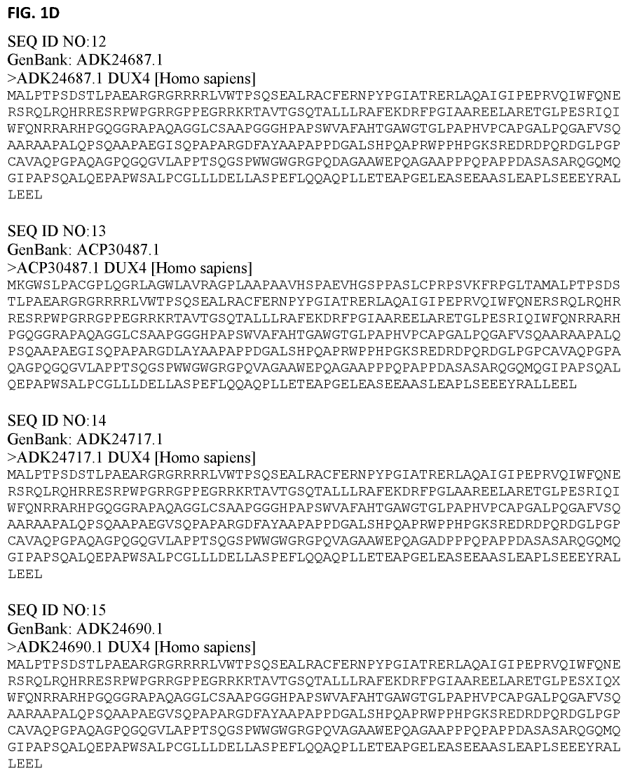

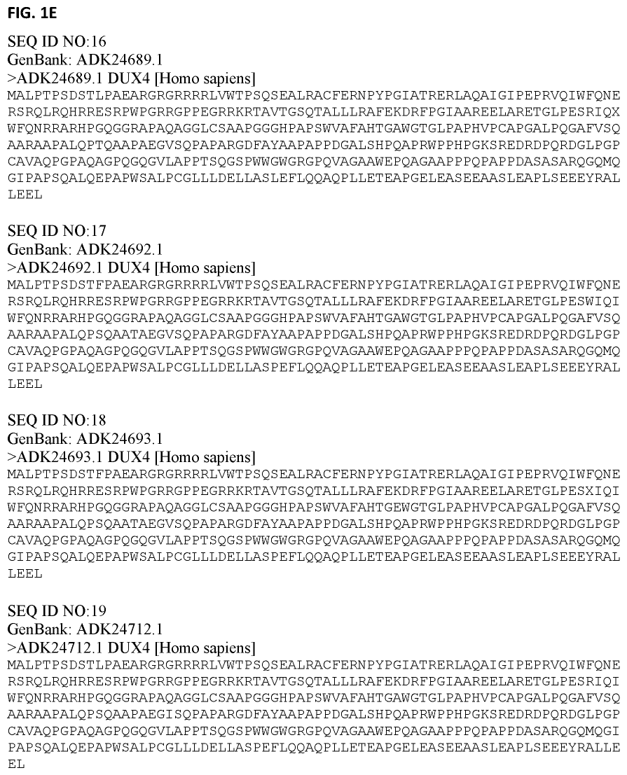

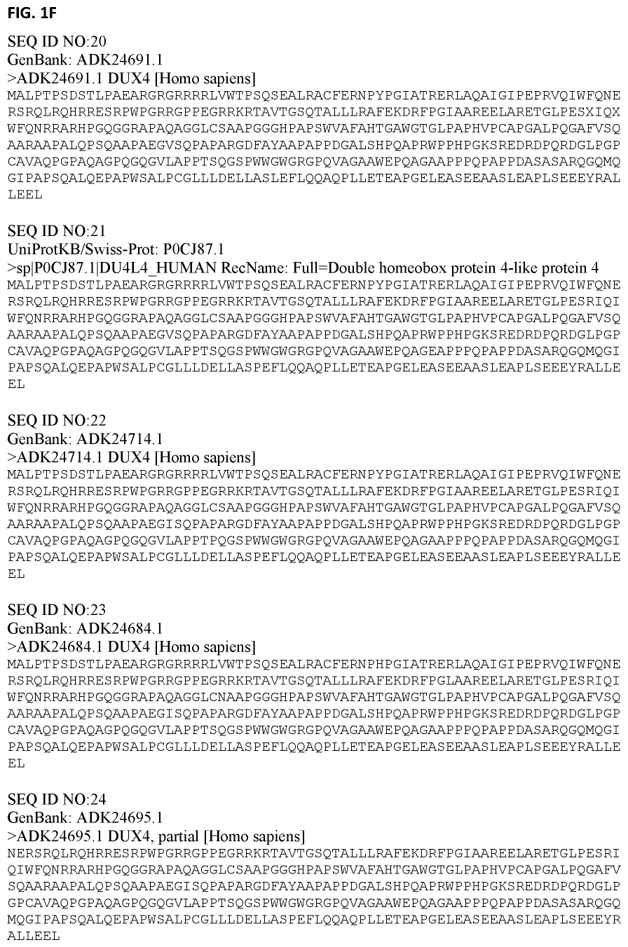

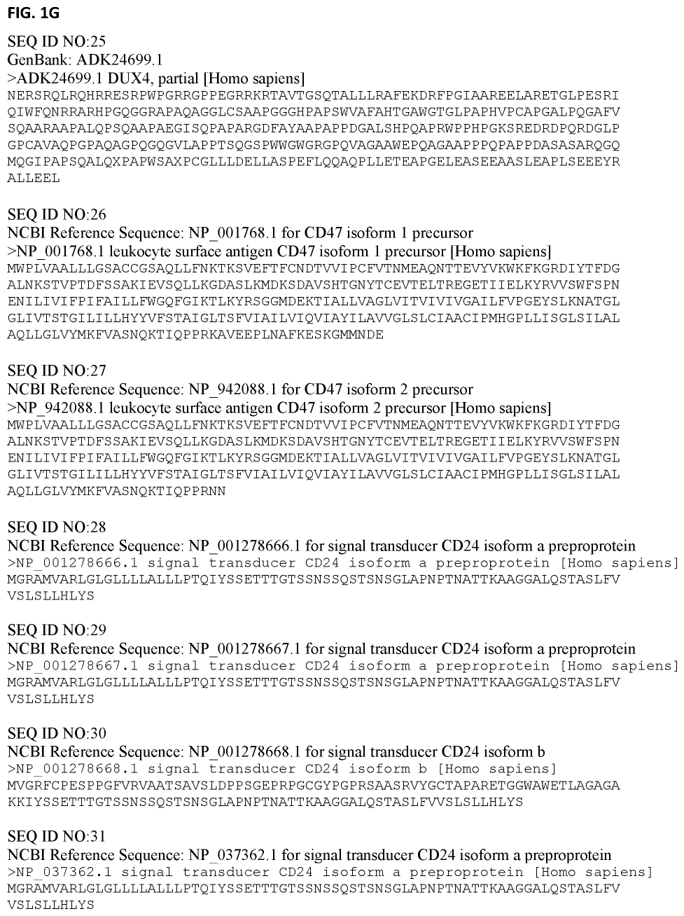

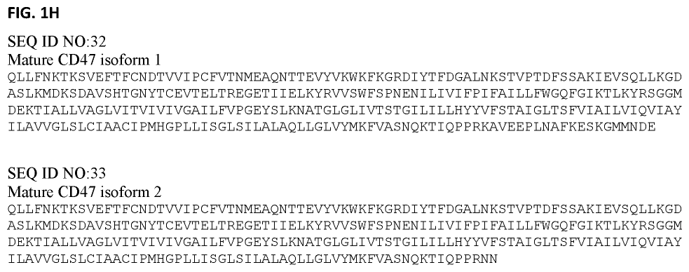

### DETAILED DESCRIPTION OF THE INVENTION

### I. Introduction

Genome editing and the generation of induced pluripotent stem cells (iPSCs) followed by the differentiation of such iPSCs remains a costly, time consuming and highly variable process, with regards to pluripotency, epigenetic status, capacity for differentiation, and genomic stability. Moreover, changes occurring during genome editing and prolonged culturing have been found to trigger an adaptive immune response, resulting in immune rejection of even autologous stem cell-derived transplants or explants. To overcome the problem of a subject's immune rejection of stem cell-derived transplants, the inventors have developed and disclose herein a hypoimmunogenic cell (e.g., a hypoimmunogenic pluripotent cell, a hypoimmunogenic differentiated cell, a hypoimmunogenic primary T cell and the like) that represents a viable source for a transplantable cell type. Such CD24 expressing cells are protected from adaptive and innate immune rejection upon administration to a recipient subject. Advantageously, the cells disclosed herein are not rejected by the recipient subject's immune system, regardless of the subject's genetic make-up. Such cells are protected from adaptive and innate immune rejection upon administration to a recipient subject.

In some embodiments, CD24 expressing hypoimmunogenic cells outlined herein are not subject to an innate immune cell rejection. In some instances, hypoimmunogenic cells are not susceptible to NK cell-mediated lysis. In some instances, hypoimmunogenic cells are not susceptible to macrophage engulfment. In some embodiments, hypoimmunogenic cells are useful as a source of universally compatible cells or tissues (e.g., universal donor cells or tissues) that are transplanted into a recipient subject with little to no immunosuppressant agent needed. Such hypoimmunogenic cells retain cell-specific characteristics and features upon transplantation.

In some embodiments, provided herein are stem cells or differentiated derivatives thereof that evade immune rejection in an MHC-mismatched allogenic recipient. In some instances, differentiated cells produced from the stem cells outlined herein evade immune rejection when administered (e.g., transplanted or grafted) to MHC-mismatched allogenic recipient. In other words, the stem cells and differentiated cells derived from such stem cells (including progeny thereof) are hypoimmunogenic. In some embodiments, hypoimmunogenic stem cells outlined herein have reduced immunogenicity (such as, at least 2.5%-99% less immunogenicity) compared to wild-type stem cells. In some instances, the hypoimmunogenic stem cells lack immunogenicity compared to wild-type stem cells. The stem cells or differentiated derivatives thereof are suitable as universal donor cells for transplantation or engrafting into a recipient patient. In some embodiments, such cells are nonimmunogenic to a patient. In some embodiments, provided herein are stem cells with reduced immunogenicity. Such stem cells retain pluripotent stem cell potential and differentiation capacity.

Methods provided are useful for inactivation or ablation of MHC class I expression and/or MHC class II expression in cells such as but not limited to pluripotent stem cells. In some embodiments, genome editing technologies utilizing rare-cutting endonucleases (e.g., the CRISPR/Cas, TALEN, zinc finger nuclease, meganuclease, and homing endonuclease systems) are also used to reduce or eliminate expression of critical immune genes (e.g., by deleting genomic DNA of critical immune genes) in human stem cells. In certain embodiments, genome editing technologies or other gene modulation technologies are used to insert tolerance-inducing factors in human cells, rendering them and the differentiated cells prepared therefrom hypoimmunogenic cells. As such, the hypoimmunogenic cells have reduced or eliminated expression of MHC I and MHC II expression. In some embodiments, the cells are nonimmunogenic (e.g., do not induce an immune response) in a recipient subject.

The genome editing techniques enable double-strand DNA breaks at desired locus sites. These controlled double-strand breaks promote homologous recombination at the specific locus sites. This process focuses on targeting specific sequences of nucleic acid molecules, such as chromosomes, with endonucleases that recognize and bind to the sequences and induce a double-stranded break in the nucleic acid molecule. The double-strand break is repaired either by an error-prone non-homologous end-joining (NHEJ) or by homologous recombination (HR).

The practice of the particular embodiments will employ, unless indicated specifically to the contrary, conventional methods of chemistry, biochemistry, organic chemistry, molecular biology, microbiology, recombinant DNA techniques, genetics, immunology, and cell biology that are within the skill of the art, many of which are described below for the purpose of illustration. Such techniques are explained fully in the literature. See, e.g., Sambrook, et al., Molecular Cloning: A Laboratory Manual (3rd Edition, 2001); Sambrook, et al., Molecular Cloning: A Laboratory Manual (2nd Edition, 1989); Maniatis et al., Molecular Cloning: A Laboratory Manual (1982); Ausubel et al., Current Protocols in Molecular Biology (John Wiley and Sons, updated July 2008); Short Protocols in Molecular Biology: A Compendium of Methods from Current Protocols in Molecular Biology, Greene Pub. Associates and Wiley-Interscience; Glover, DNA Cloning: A Practical Approach, vol. I & II (IRL Press, Oxford, 1985); Anand, Techniques for the Analysis of Complex Genomes, (Academic Press, New York, 1992); Transcription and Translation (B. Hames & S. Higgins, Eds., 1984); Perbal, A Practical Guide to Molecular Cloning (1984); Harlow and Lane, Antibodies, (Cold Spring Harbor Laboratory Press, Cold Spring Harbor, N.Y., 1998) Current Protocols in Immunology Q. E. Coligan, A. M. Kruisbeek, D. H. Margulies, E. M. Shevach and W. Strober, eds., 1991); Annual Review of Immunology; as well as monographs in journals such as Advances in Immunology.

### II. Definitions

As used herein to characterize a cell, the term “hypoimmunogenic” generally means that such cell is less prone to immune rejection by a subject into which such cells are transplanted. For example, relative to an unaltered or unmodified wild-type cell, such a hypoimmunogenic cell may be about 2.5%, 5%, 10%, 20%, 30%, 40%, 50%, 60%, 70%, 80%, 90%, 95%, 97.5%, 99% or more less prone to immune rejection by a subject into which such cells are transplanted. In some aspects, genome editing technologies are used to modulate the expression of MHC I and MHC II genes, and thus, generate a hypoimmunogenic cell. In some embodiments, a hypoimmunogenic cell evades immune rejection in an MHC-mismatched allogenic recipient. In some instance, differentiated cells produced from the hypoimmunogenic stem cells outlined herein evade immune rejection when administered (e.g., transplanted or grafted) to an MHC-mismatched allogenic recipient. In some embodiments, a hypoimmunogenic cell is protected from T cell-mediated adaptive immune rejection and/or innate immune cell rejection.

Hypoimmunogencity of a cell can be determined by evaluating the immunogenicity of the cell such as the cell's ability to elicit adaptive and innate immune responses. Such immune response can be measured using assays recognized by those skilled in the art. In some embodiments, an immune response assay measures the effect of a hypoimmunogenic cell on T cell proliferation, T cell activation, T cell killing, NK cell proliferation, NK cell activation, and macrophage activity. In some cases, hypoimmunogenic cells and derivatives thereof undergo decreased killing by T cells and/or NK cells upon administration to a subject. In some instances, the cells and derivatives thereof show decreased macrophage engulfment compared to an unmodified or wildtype cell. In some embodiments, a hypoimmunogenic cell elicits a reduced or diminished immune response in a recipient subject compared to a corresponding unmodified wild-type cell. In some embodiments, a hypoimmunogenic cell is nonimmunogenic or fails to elicit an immune response in a recipient subject.

“Immunosuppressive factor” or “immune regulatory factor” or “tolerogenic factor” as used herein include hypoimmunity factors, complement inhibitors, and other factors that modulate or affect the ability of a cell to be recognized by the immune system of a host or recipient subject upon administration, transplantation, or engraftment.

“Immune signaling factor” as used herein refers to, in some cases, a molecule, protein, peptide and the like that activates immune signaling pathways.

“Safe harbor locus” as used herein refers to a gene locus that allows safe expression of a transgene or an exogenous gene. Exemplary “safe harbor” loci include a CCR5 gene, a CXCR4 gene, a PPP1R12C (also known as AAVS1) gene, an albumin gene, a SHS231 locus, a CLYBL gene, and a Rosa gene (e.g., ROSA26).

A “gene,” for the purposes of the present disclosure, includes a DNA region encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

“Gene expression” refers to the conversion of the information, contained in a gene, into a gene product. A gene product can be the direct transcriptional product of a gene (e.g., mRNA, tRNA, rRNA, antisense RNA, ribozyme, structural RNA or any other type of RNA) or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristoylation, and glycosylation.

“Modulation” of gene expression refers to a change in the expression level of a gene. Modulation of expression can include, but is not limited to, gene activation and gene repression. Modulation may also be complete, i.e. wherein gene expression is totally inactivated or is activated to wildtype levels or beyond; or it may be partial, wherein gene expression is partially reduced, or partially activated to some fraction of wildtype levels.

The term “operatively linked” or “operably linked” are used interchangeably with reference to a juxtaposition of two or more components (such as sequence elements), in which the components are arranged such that both components function normally and allow the possibility that at least one of the components can mediate a function that is exerted upon at least one of the other components. By way of illustration, a transcriptional regulatory sequence, such as a promoter, is operatively linked to a coding sequence if the transcriptional regulatory sequence controls the level of transcription of the coding sequence in response to the presence or absence of one or more transcriptional regulatory factors. A transcriptional regulatory sequence is generally operatively linked in cis with a coding sequence, but need not be directly adjacent to it. For example, an enhancer is a transcriptional regulatory sequence that is operatively linked to a coding sequence, even though they are not contiguous.

A “vector” or “construct” is capable of transferring gene sequences to target cells. Typically, “vector construct,” “expression vector,” and “gene transfer vector,” mean any nucleic acid construct capable of directing the expression of a gene of interest and which can transfer gene sequences to target cells. Thus, the term includes cloning, and expression vehicles, as well as integrating vectors. Methods for the introduction of vectors or constructs into cells are known to those of skill in the art and include, but are not limited to, lipid-mediated transfer (i.e., liposomes, including neutral and cationic lipids), electroporation, direct injection, cell fusion, particle bombardment, calcium phosphate co-precipitation, DEAE-dextran-mediated transfer and viral vector-mediated transfer.

“Pluripotent stem cells” as used herein have the potential to differentiate into any of the three germ layers: endoderm (e.g., the stomach linking, gastrointestinal tract, lungs, etc.), mesoderm (e.g., muscle, bone, blood, urogenital tissue, etc.) or ectoderm (e.g. epidermal tissues and nervous system tissues). The term “pluripotent stem cells,” as used herein, also encompasses “induced pluripotent stem cells”, or “iPSCs”, or a type of pluripotent stem cell derived from a non-pluripotent cell. In some embodiments, a pluripotent stem cell is produced or generated from a cell that is not a pluripotent cell. In other words, pluripotent stem cells can be direct or indirect progeny of a non-pluripotent cell. Examples of parent cells include somatic cells that have been reprogrammed to induce a pluripotent, undifferentiated phenotype by various means. Such iPS” or “iPSC” cells can be created by inducing the expression of certain regulatory genes or by the exogenous application of certain proteins. Methods for the induction of iPS cells are known in the art and are further described below. (See, e.g., Zhou et al., Stem Cells 27 (11): 2667-74 (2009); Huangfu et al., Nature Biotechnol. 26 (7): 795 (2008); Woltjen et al., Nature 458 (7239): 766-770 (2009); and Zhou et al., Cell Stem Cell 8:381-384 (2009); each of which is incorporated by reference herein in their entirety.) The generation of induced pluripotent stem cells (iPSCs) is outlined below. As used herein, “hiPSCs” are human induced pluripotent stem cells.

By “HLA” or “human leukocyte antigen” complex is a gene complex encoding the major histocompatibility complex (MHC) proteins in humans. These cell-surface proteins that make up the HLA complex are responsible for the regulation of the immune response to antigens. In humans, there are two MHCs, class I and class II, “HLA-I” and “HLA-II”. HLA-I includes three proteins, HLA-A, HLA-B and HLA-C, which present peptides from the inside of the cell, and antigens presented by the HLA-I complex attract killer T-cells (also known as CD8+ T-cells or cytotoxic T cells). The HLA-I proteins are associated with β-2 microglobulin (B2M). HLA-II includes five proteins, HLA-DP, HLA-DM, HLA-DOB, HLA-DQ and HLA-DR, which present antigens from outside the cell to T lymphocytes. This stimulates CD4+ cells (also known as T-helper cells). It should be understood that the use of either “MHC” or “HLA” is not meant to be limiting, as it depends on whether the genes are from humans (HLA) or murine (MHC). Thus, as it relates to mammalian cells, these terms may be used interchangeably herein.

As used herein, the terms “grafting”, “administering,” “introducing”, “implanting” and “transplanting” as well as grammatical variations thereof are used interchangeably in the context of the placement of cells (e.g. cells described herein) into a subject, by a method or route which results in at least partial localization of the introduced cells at a desired site. The cells can be implanted directly to the desired site, or alternatively be administered by any appropriate route which results in delivery to a desired location in the subject where at least a portion of the implanted cells or components of the cells remain viable. The period of viability of the cells after administration to a subject can be as short as a few hours, e. g. twenty-four hours, to a few days, to as long as several years. In some embodiments, the cells can also be administered (e.g., injected) a location other than the desired site, such as in the brain or subcutaneously, for example, in a capsule to maintain the implanted cells at the implant location and avoid migration of the implanted cells.

The terms “treat”, “treating”, “treatment”, etc., as applied to an isolated cell, include subjecting the cell to any kind of process or condition or performing any kind of manipulation or procedure on the cell. As applied to a subject, the terms refer to administering a cell or population of cells in which a target polynucleotide sequence (e.g., B2M) has been altered ex vivo according to the methods described herein to an individual. The individual is usually ill or injured, or at increased risk of becoming ill relative to an average member of the population and in need of such attention, care, or management.

As used herein, the term “treating” and “treatment” refers to administering to a subject an effective amount of cells with target polynucleotide sequences altered ex vivo according to the methods described herein so that the subject has a reduction in at least one symptom of the disease or an improvement in the disease, for example, beneficial or desired clinical results. For purposes of this invention, beneficial or desired clinical results include, but are not limited to, alleviation of one or more symptoms, diminishment of extent of disease, stabilized (i.e., not worsening) state of disease, delay or slowing of disease progression, amelioration or palliation of the disease state, and remission (whether partial or total), whether detectable or undetectable. Treating can refer to prolonging survival as compared to expected survival if not receiving treatment. Thus, one of skill in the art realizes that a treatment may improve the disease condition, but may not be a complete cure for the disease. As used herein, the term “treatment” includes prophylaxis. Alternatively, treatment is “effective” if the progression of a disease is reduced or halted. “Treatment” can also mean prolonging survival as compared to expected survival if not receiving treatment. Those in need of treatment include those already diagnosed with a disorder associated with expression of a polynucleotide sequence, as well as those likely to develop such a disorder due to genetic susceptibility or other factors.

By “treatment”, “prevention” or “amelioration” of a disease or disorder is meant delaying or preventing the onset of such a disease or disorder, reversing, alleviating, ameliorating, inhibiting, slowing down or stopping the progression, aggravation or deterioration the progression or severity of a condition associated with such a disease or disorder. In one embodiment, the symptoms of a disease or disorder are alleviated by at least 5%, at least 10%, at least 20%, at least 30%, at least 40%, or at least 50%.

The term “cancer” as used herein is defined as a hyperproliferation of cells whose unique trait (e.g., loss of normal controls) results in unregulated growth, lack of differentiation, local tissue invasion, and metastasis. With respect to the inventive methods, the cancer can be any cancer, including any of acute lymphocytic cancer, acute myeloid leukemia, alveolar rhabdomyosarcoma, bladder cancer, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia, chronic myeloid cancer, colon cancer, esophageal cancer, cervical cancer, fibrosarcoma, gastrointestinal carcinoid tumor, Hodgkin lymphoma, hypopharynx cancer, kidney cancer, larynx cancer, leukemia, liquid tumors, liver cancer, lung cancer, lymphoma, malignant mesothelioma, mastocytoma, melanoma, multiple myeloma, nasopharynx cancer, non-Hodgkin lymphoma, ovarian cancer, pancreatic cancer, peritoneum, omentum, and mesentery cancer, pharynx cancer, prostate cancer, rectal cancer, renal cancer, skin cancer, small intestine cancer, soft tissue cancer, solid tumors, stomach cancer, testicular cancer, thyroid cancer, ureter cancer, and urinary bladder cancer. As used herein, the term “tumor” refers to an abnormal growth of cells or tissues of the malignant type, unless otherwise specifically indicated and does not include a benign type tissue.

The term “chronic infectious disease” refers to a disease caused by an infectious agent wherein the infection has persisted. Such a disease may include hepatitis (A, B, or C), herpes virus (e.g., VZV, HSV-1, HSV-6, HSV-II, CMV, and EBV), and HIV/AIDS. Non-viral examples may include chronic fungal diseases such Aspergillosis, Candidiasis, Coccidioidomycosis, and diseases associated with Cryptococcus and Histoplasmosis. None limiting examples of chronic bacterial infectious agents may be Chlamydia pneumoniae, Listeria monocytogenes, and Mycobacterium tuberculosis. In some embodiments, the disorder is human immunodeficiency virus (HIV) infection. In some embodiments, the disorder is acquired immunodeficiency syndrome (AIDS).

The term “autoimmune disease” refers to any disease or disorder in which the subject mounts a destructive immune response against its own tissues. Autoimmune disorders can affect almost every organ system in the subject (e.g., human), including, but not limited to, diseases of the nervous, gastrointestinal, and endocrine systems, as well as skin and other connective tissues, eyes, blood and blood vessels. Examples of autoimmune diseases include, but are not limited to Hashimoto's thyroiditis, Systemic lupus erythematosus, Sjogren's syndrome, Graves' disease, Scleroderma, Rheumatoid arthritis, Multiple sclerosis, Myasthenia gravis and Diabetes.

In additional or alternative aspects, the present invention contemplates altering target polynucleotide sequences in any manner which is available to the skilled artisan, e.g., utilizing a nuclease system such as a TAL effector nuclease (TALEN) or zinc finger nuclease (ZFN) system. It should be understood that although examples of methods utilizing CRISPR/Cas (e.g., Cas9 and Cpf1) and TALEN are described in detail herein, the invention is not limited to the use of these methods/systems. Other methods of targeting, e.g., B2M, to reduce or ablate expression in target cells known to the skilled artisan can be utilized herein.

The methods of the present invention can be used to alter a target polynucleotide sequence in a cell. The present invention contemplates altering target polynucleotide sequences in a cell for any purpose. In some embodiments, the target polynucleotide sequence in a cell is altered to produce a mutant cell. As used herein, a “mutant cell” refers to a cell with a resulting genotype that differs from its original genotype. In some instances, a “mutant cell” exhibits a mutant phenotype, for example when a normally functioning gene is altered using the CRISPR/Cas systems of the present invention. In other instances, a “mutant cell” exhibits a wild-type phenotype, for example when a CRISPR/Cas system of the present invention is used to correct a mutant genotype. In some embodiments, the target polynucleotide sequence in a cell is altered to correct or repair a genetic mutation (e.g., to restore a normal phenotype to the cell). In some embodiments, the target polynucleotide sequence in a cell is altered to induce a genetic mutation (e.g., to disrupt the function of a gene or genomic element).

In some embodiments, the alteration is an indel. As used herein, “indel” refers to a mutation resulting from an insertion, deletion, or a combination thereof. As will be appreciated by those skilled in the art, an indel in a coding region of a genomic sequence will result in a frameshift mutation, unless the length of the indel is a multiple of three. In some embodiments, the alteration is a point mutation. As used herein, “point mutation” refers to a substitution that replaces one of the nucleotides. A CRISPR/Cas system of the present invention can be used to induce an indel of any length or a point mutation in a target polynucleotide sequence.

As used herein, “knock out” includes deleting all or a portion of the target polynucleotide sequence in a way that interferes with the function of the target polynucleotide sequence. For example, a knock out can be achieved by altering a target polynucleotide sequence by inducing an indel in the target polynucleotide sequence in a functional domain of the target polynucleotide sequence (e.g., a DNA binding domain). Those skilled in the art will readily appreciate how to use the CRISPR/Cas systems of the present invention to knock out a target polynucleotide sequence or a portion thereof based upon the details described herein.

In some embodiments, the alteration results in a knock out of the target polynucleotide sequence or a portion thereof. Knocking out a target polynucleotide sequence or a portion thereof using a CRISPR/Cas system of the present invention can be useful for a variety of applications. For example, knocking out a target polynucleotide sequence in a cell can be performed in vitro for research purposes. For ex vivo purposes, knocking out a target polynucleotide sequence in a cell can be useful for treating or preventing a disorder associated with expression of the target polynucleotide sequence (e.g., by knocking out a mutant allele in a cell ex vivo and introducing those cells comprising the knocked out mutant allele into a subject).

By “knock in” herein is meant a process that adds a genetic function to a host cell. This causes increased levels of the knocked in gene product, e.g., an RNA or encoded protein. As will be appreciated by those in the art, this can be accomplished in several ways, including adding one or more additional copies of the gene to the host cell or altering a regulatory component of the endogenous gene increasing expression of the protein is made. This may be accomplished by modifying the promoter, adding a different promoter, adding an enhancer, or modifying other gene expression sequences.

In some embodiments, an alteration or modification described herein results in reduced expression of a target or selected polynucleotide sequence. In some embodiments, an alteration or modification described herein results in reduced expression of a target or selected polypeptide sequence.

In some embodiments, an alteration or modification described herein results in increased expression of a target or selected polynucleotide sequence. In some embodiments, an alteration or modification described herein results in increased expression of a target or selected polypeptide sequence.

The terms “decrease,” “reduced,” “reduction,” and “decrease” are all used herein generally to mean a decrease by a statistically significant amount. However, for avoidance of doubt, decrease,” “reduced,” “reduction,” “decrease” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

As used herein, the term “exogenous” in intended to mean that the referenced molecule or the referenced polypeptide is introduced into the cell of interest. The polypeptide can be introduced, for example, by introduction of an encoding nucleic acid into the genetic material of the cells such as by integration into a chromosome or as non-chromosomal genetic material such as a plasmid or expression vector. Therefore, the term as it is used in reference to expression of an encoding nucleic acid refers to introduction of the encoding nucleic acid in an expressible form into the cell. An “exogenous” molecule is a molecule, construct, factor and the like that is not normally present in a cell, but can be introduced into a cell by one or more genetic, biochemical or other methods. “Normal presence in the cell” is determined with respect to the particular developmental stage and environmental conditions of the cell. Thus, for example, a molecule that is present only during embryonic development of neurons is an exogenous molecule with respect to an adult neuron cell. An exogenous molecule can comprise, for example, a functioning version of a malfunctioning endogenous molecule or a malfunctioning version of a normally-functioning endogenous molecule.

An exogenous molecule or factor can be, among other things, a small molecule, such as is generated by a combinatorial chemistry process, or a macromolecule such as a protein, nucleic acid, carbohydrate, lipid, glycoprotein, lipoprotein, polysaccharide, any modified derivative of the above molecules, or any complex comprising one or more of the above molecules. Nucleic acids include DNA and RNA, can be single- or double-stranded; can be linear, branched or circular; and can be of any length. Nucleic acids include those capable of forming duplexes, as well as triplex-forming nucleic acids. See, for example, U.S. Pat. Nos. 5,176,996 and 5,422,251. Proteins include, but are not limited to, DNA-binding proteins, transcription factors, chromatin remodeling factors, methylated DNA binding proteins, polymerases, methylases, demethylases, acetylases, deacetylases, kinases, phosphatases, integrases, recombinases, ligases, topoisomerases, gyrases and helicases.

The term “endogenous” refers to a referenced molecule or polypeptide that is present in the cell. Similarly, the term when used in reference to expression of an encoding nucleic acid refers to expression of an encoding nucleic acid contained within the cell and not exogenously introduced.

The term percent “identity,” in the context of two or more nucleic acid or polypeptide sequences, refers to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared. For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information.

The terms “subject” and “individual” are used interchangeably herein, and refer to an animal, for example, a human from whom cells can be obtained and/or to whom treatment, including prophylactic treatment, with the cells as described herein, is provided. For treatment of those infections, conditions or disease states, which are specific for a specific animal such as a human subject, the term subject refers to that specific animal. The “non-human animals” and “non-human mammals” as used interchangeably herein, includes mammals such as rats, mice, rabbits, sheep, cats, dogs, cows, pigs, and non-human primates. The term “subject” also encompasses any vertebrate including but not limited to mammals, reptiles, amphibians and fish. However, advantageously, the subject is a mammal such as a human, or other mammals such as a domesticated mammal, e.g. dog, cat, horse, and the like, or production mammal, e.g. cow, sheep, pig, and the like.

It is noted that the claims may be drafted to exclude any optional element. As such, this statement is intended to serve as antecedent basis for use of such exclusive terminology as “solely,” “only,” and the like in connection with the recitation of claim elements, or use of a “negative” limitation. As will be apparent to those of skill in the art upon reading this disclosure, each of the individual embodiments described and illustrated herein has discrete components and features readily separated from or combined with the features of any of the other several embodiments without departing from the scope or spirit of the invention. Any recited method may be carried out in the order of events recited or in any other order that is logically possible. Although any methods and materials similar or equivalent to those described herein may also be used in the practice or testing of the invention, representative illustrative methods and materials are now described.

As described in the present invention, the following terms will be employed, and are defined as indicated below.

Before the invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention. Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number, which, in the context presented, provides the substantial equivalent of the specifically recited number.

All publications, patents, and patent applications cited in this specification are incorporated herein by reference to the same extent as if each individual publication, patent, or patent application were specifically and individually indicated to be incorporated by reference. Furthermore, each cited publication, patent, or patent application is incorporated herein by reference to disclose and describe the subject matter in connection with which the publications are cited. The citation of any publication is for its disclosure prior to the filing date and should not be construed as an admission that the invention described herein is not entitled to antedate such publication by virtue of prior invention. Further, the dates of publication provided might be different from the actual publication dates, which may need to be independently confirmed.

### III. Detailed Description of the Embodiments

### A. Hypoimmunogenic Cells

Provided herein are cells comprising a modification for increasing expression of CD24 and a modification of one or more targeted polynucleotide sequences that regulates the expression of MHC class I and/or MHC class II human leukocyte antigens. In some embodiments, the cells comprise an exogenous CD24 polypeptide. In some embodiments, the cells also include a modification to increase expression of one or more polypeptides selected from the group consisting of CD47, DUX4, CD27, CD35, CD46, CD55, CD59, CD200, HLA-C, HLA-E, HLA-E heavy chain, HLA-G, PD-L1, IDO1, CTLA4, C1-Inhibitor, IL-10, IL-35, FASL, CCL21, Mfge8, and Serpinb95. In some embodiments, the cells described comprise exogenous CD24 and CD47 polypeptides, exogenous CD24 and DUX4 polypeptides, exogenous CD24 and CD27 polypeptides, exogenous CD24 and CD35 polypeptides, exogenous CD24 and CD46 polypeptides, exogenous CD24 and CD55 polypeptides, exogenous CD24 and CD59 polypeptides, exogenous CD24 and CD200 polypeptides, exogenous CD24 and HLA-C polypeptides, exogenous CD24 and HLA-E polypeptides, exogenous CD24 and HLA-E heavy chain polypeptides, exogenous CD24 and HLA-G polypeptides, exogenous CD24 and PD-L1 polypeptides, exogenous CD24 and IDO1 polypeptides, exogenous CD24 and CTLA4 polypeptides, exogenous CD24 and C1-Inhibitor polypeptides, exogenous CD24 and IL-10 polypeptides, exogenous CD24 and IL-35 polypeptides, exogenous CD24 and FASL polypeptides, exogenous CD24 and CCL21 polypeptides, exogenous CD24 and Mfge8 polypeptides, and exogenous CD24 and Serpinb95 polypeptides, and the like.

In some embodiments, the cells comprise a genomic modification of one or more targeted polynucleotide sequences that regulates the expression of MHC I and/or MHC II. In some aspects, a genetic editing system is used to modify one or more targeted polynucleotide sequences. In some embodiments, the targeted polynucleotide sequence is one or more selected from the group consisting of B2M, CIITA, and NLRC5. In certain embodiments, the genome of the cells have been altered to reduce or delete critical components of HLA expression.

In some aspects, the present disclosure provides a cell (e.g., a stem cell, pluripotent stem cell, induced pluripotent stem cell, differentiated cell derived or produced from such a stem cell, hematopoietic stem cell, primary T cell, chimeric antigen receptor (CAR) T cell, and any progeny thereof) or population thereof comprising a genome in which a gene has been edited to delete a contiguous stretch of genomic DNA, thereby reducing or eliminating surface expression of MHC class I molecules in the cell or population thereof. In certain aspects, the present disclosure provides a cell (e.g., a stem cell, pluripotent stem cell, induced pluripotent stem cell, differentiated cell derived or produced from such a stem cell, hematopoietic stem cell, primary T cell, chimeric antigen receptor (CAR) T cell, and any progeny thereof) or population thereof comprising a genome in which a gene has been edited to delete a contiguous stretch of genomic DNA, thereby reducing or eliminating surface expression of MHC class II molecules in the cell or population thereof. In particular aspects, the present disclosure provides a cell (e.g., a stem cell, pluripotent stem cell, induced pluripotent stem cell, differentiated cell derived or produced from such a stem cell, hematopoietic stem cell, primary T cell, chimeric antigen receptor (CAR) T cell, and any progeny thereof) or population thereof comprising a genome in which one or more genes has been edited to delete a contiguous stretch of genomic DNA, thereby reducing or eliminating surface expression of MHC class I and II molecules in the cell or population thereof. In particular aspects, the present disclosure provides a stem cell (e.g., pluripotent stem cell or induced pluripotent stem cell) or population thereof comprising a genome in which one or more genes has been edited to delete a contiguous stretch of genomic DNA, thereby reducing or eliminating surface expression of MHC class I and II molecules in the cell or population thereof.

In certain embodiments, the expression of MHC I or MHC II is modulated by targeting and deleting a contiguous stretch of genomic DNA thereby reducing or eliminating expression of a target gene selected from the group consisting of B2M, CIITA, and NLRC5.

In some embodiments, the cells and methods described herein include genomically editing human cells to cleave CIITA gene sequences as well as editing the genome of such cells to alter one or more additional target polynucleotide sequences such as, but not limited to, B2M and NLRC5. In some embodiments, the cells and methods described herein include genomically editing human cells to cleave B2M gene sequences as well as editing the genome of such cells to alter one or more additional target polynucleotide sequences such as, but not limited to, CIITA and NLRC5. In some embodiments, the cells and methods described herein include genomically editing human cells to cleave NLRC5 gene sequences as well as editing the genome of such cells to alter one or more additional target polynucleotide sequences such as, but not limited to, B2M and CIITA.

In certain embodiments, the expression of MHC I is modulated by overexpressing or increasing the expression of DUX4. In some cases, the polynucleotide sequence encoding DUX4 is a codon altered sequence comprising one or more base substitutions to reduce the total number of CpG sites while preserving the DUX4 protein sequence. In some instances, the codon altered sequence is SEQ ID NO:1. In other cases, the polynucleotide sequence encoding DUX4 is a nucleotide sequence encoding a polypeptide sequence having at least 90% (e.g., 90%, 91%, 92%, 93%, 94%, 95%, 96%, 97%, 98%, 99%, or more) sequence identity to a sequence selected from the group consisting of SEQ ID NOS:2-25. In some cases, the polynucleotide sequence encoding DUX4 is a nucleotide sequence encoding a polypeptide having a sequence selected from the group consisting of SEQ ID NOS:2-25. In some embodiments, cells described herein that have increased expression of DUX4 also overexpression CD24.

In some embodiments, the cells described herein include, but are not limited to, pluripotent stem cells, induced pluripotent stem cells, differentiated cells derived or produced from such stem cells, hematopoietic stem cells, primary T cells, chimeric antigen receptor (CAR) T cells, and any progeny thereof. In some embodiments, the present disclosure provides a stem cell (e.g., a hypoimmunogenic stem cell, a pluripotent stem cell, an adult stem cell, and a hematopoietic stem cell) or a population thereof that has been modified as described herein.

In some embodiments, the primary T cells are selected from a group that includes cytotoxic T-cells, helper T-cells, memory T-cells, regulatory T-cells, tumor infiltrating lymphocytes, and combinations thereof.

In some embodiments, the primary T cells are from a pool of primary T cells from one or more donor subjects that are different than the recipient subject (e.g., the patient administered the cells). The primary T cells can be obtained from 1, 2, 3, 4, 5, 6, 7, 8, 9, 10, 20, 50, 100 or more donor subjects and pooled together. In some embodiments, the primary T cells are harvested from one or a plurality of individuals, and in some instances, the primary T cells or the pool of primary T cells are cultured in vitro. In some embodiments, the primary T cells or the pool of primary T cells are engineered to exogenously express CD24, and in some instances, also CD47 and cultured in vitro.

In certain embodiments, the primary T cells or the pool of primary T cells are engineered to express a chimeric antigen receptor (CAR). The CAR can be any known to those skilled in the art. Useful CARs include those that bind an antigen selected from a group that includes CD19, CD38, CD123, CD138, and BCMA. In some cases, the CAR is the same or equivalent to those used in FDA-approved CAR-T cell therapies such as, but not limited to, those used in tisagenlecleucel and axicabtagene ciloleucel, or others under investigation in clinical trials.

In some embodiments, the primary T cells or the pool of primary T cells are engineered to exhibit reduced expression of an endogenous T cell receptor compared to unmodified primary T cells. In certain embodiments, the primary T cells or the pool of primary T cells are engineered to exhibit reduced expression of CTLA4, PD1, or both CTLA4 and PD1, as compared to unmodified primary T cells. Methods of genetically modifying a cell including a T cell are described in detail, for example, in WO2016183041, the disclosure is herein incorporated by reference in its entirety including the tables, appendices, sequence listing and figures.

In some embodiments, the CAR T cells comprise a CAR selected from a group including: (a) a first generation CAR comprising an antigen binding domain, a transmembrane domain, and a signaling domain; (b) a second generation CAR comprising an antigen binding domain, a transmembrane domain, and at least two signaling domains; (c) a third generation CAR comprising an antigen binding domain, a transmembrane domain, and at least three signaling domains; and (d) a fourth generation CAR comprising an antigen binding domain, a transmembrane domain, three or four signaling domains, and a domain which upon successful signaling of the CAR induces expression of a cytokine gene.

In some embodiments, the antigen binding domain of the CAR is selected from a group including, but not limited to, (a) an antigen binding domain targets an antigen characteristic of a neoplastic cell; (b) an antigen binding domain that targets an antigen characteristic of a T cell; (c) an antigen binding domain targets an antigen characteristic of an autoimmune or inflammatory disorder; (d) an antigen binding domain that targets an antigen characteristic of senescent cells; (e) an antigen binding domain that targets an antigen characteristic of an infectious disease; and (0 an antigen binding domain that binds to a cell surface antigen of a cell.

In some embodiments, the antigen binding domain is selected from a group that includes an antibody, an antigen-binding portion or fragment thereof, an scFv, and a Fab. In some embodiments, the antigen binding domain binds to CD19 or BCMA. In some embodiments, the antigen binding domain is an anti-CD19 scFv such as but not limited to FMC63.

In some embodiments, the transmembrane domain of the CAR comprises one selected from a group that includes a transmembrane region of TCRα, TCRβ, TCRζ, CD3ε, CD3γ, CD3δ, CD3ζ, CD4, CD5, CD8α, CD8β, CD9, CD16, CD28, CD45, CD22, CD33, CD34, CD37, CD40, CD40L/CD154, CD45, CD64, CD80, CD86, OX40/CD134, 4-1BB/CD137, CD154, FcεRIγ, VEGFR2, FAS, FGFR2B, and functional variant thereof.

In some embodiments, the signaling domain(s) of the CAR comprises a costimulatory domain(s). For instance, a signaling domain can contain a costimulatory domain. Or, a signaling domain can contain one or more costimulatory domains. In certain embodiments, the signaling domain comprises a costimulatory domain. In other embodiments, the signaling domains comprise costimulatory domains. In some cases, when the CAR comprises two or more costimulatory domains, two costimulatory domains are not the same. In some embodiments, the costimulatory domains comprise two costimulatory domains that are not the same. In some embodiments, the costimulatory domain enhances cytokine production, CAR T cell proliferation, and/or CAR T cell persistence during T cell activation. In some embodiments, the costimulatory domains enhance cytokine production, CAR T cell proliferation, and/or CAR T cell persistence during T cell activation.

As described herein, a fourth generation CAR can contain an antigen binding domain, a transmembrane domain, three or four signaling domains, and a domain which upon successful signaling of the CAR induces expression of a cytokine gene. In some instances, the cytokine gene is an endogenous or exogenous cytokine gene of the hypoimmunogenic cells. In some cases, the cytokine gene encodes a pro-inflammatory cytokine. In some embodiments, the pro-inflammatory cytokine is selected from a group that includes IL-1, IL-2, IL-9, IL-12, IL-18, TNF, IFN-gamma, and a functional fragment thereof. In some embodiments, the domain which upon successful signaling of the CAR induces expression of the cytokine gene comprises a transcription factor or functional domain or fragment thereof.

In some embodiments, the CAR comprises a CD3 zeta domain or an immunoreceptor tyrosine-based activation motif (ITAM), or functional variant thereof. In some embodiments, the CAR comprises (i) a CD3 zeta domain, or an immunoreceptor tyrosine-based activation motif (ITAM), or functional variant thereof; and (ii) a CD28 domain, or a 4-1BB domain, or functional variant thereof. In other embodiments, the CAR comprises (i) a CD3 zeta domain, or an immunoreceptor tyrosine-based activation motif (ITAM), or functional variant thereof; (ii) a CD28 domain or functional variant thereof; and (iii) a 4-1BB domain, or a CD134 domain, or functional variant thereof. In certain embodiments, the CAR comprises (i) a CD3 zeta domain, or an immunoreceptor tyrosine-based activation motif (ITAM), or functional variant thereof; (ii) a CD28 domain or functional variant thereof; (iii) a 4-1BB domain, or a CD134 domain, or functional variant thereof; and (iv) a cytokine or costimulatory ligand transgene. In some embodiments, the CAR comprises a (i) an anti-CD19 scFv; (ii) a CD8a hinge and transmembrane domain or functional variant thereof; (iii) a 4-1BB costimulatory domain or functional variant thereof; and (iv) a CD3 signaling domain or functional variant thereof.

Methods for introducing a CAR construct or producing a CAR-T cells are well known to those skilled in the art. Detailed descriptions are found, for example, in Vormittag et al., Curr Opin Biotechnol, 2018, 53, 162-181; and Eyquem et al., Nature, 2017, 543, 113-117.

In some embodiments, the cells derived from primary T cells comprise reduced expression of an endogenous T cell receptor, for example by disruption of an endogenous T cell receptor gene (e.g., T cell receptor alpha constant region (TRAC) or T cell receptor beta constant region (TRBC)). In some embodiments, an exogenous nucleic acid encoding a polypeptide as disclosed herein (e.g., a chimeric antigen receptor, CD24, CD47, or another tolerogenic factor disclosed herein) is inserted at the disrupted T cell receptor gene.

In some embodiments, the cells derived from primary T cells comprise reduced expression of cytotoxic T-lymphocyte-associated protein 4 (CTLA4) and/or programmed cell death (PD1). Methods of reducing or eliminating expression of CTLA4, PD1 and both CTLA4 and PD1 can include any recognized by those skilled in the art, such as but not limited to, genetic modification technologies that utilize rare-cutting endonucleases and RNA silencing or RNA interference technologies. Non-limiting examples of a rare-cutting endonuclease include any Cas protein, TALEN, zinc finger nuclease, meganuclease, and homing endonuclease.

In some embodiments, the population of engineered cells described elicits a reduced level of immune activation or no immune activation upon administration to a recipient subject. In some embodiments, the cells elicit a reduced level of systemic TH1 activation or no systemic TH1 activation in a recipient subject. In some embodiments, the cells elicit a reduced level of immune activation of peripheral blood mononuclear cells (PBMCs) or no immune activation of PBMCs in a recipient subject. In some embodiments, the cells elicit a reduced level of donor-specific IgG antibodies or no donor specific IgG antibodies against the cells upon administration to a recipient subject. In some embodiments, the cells elicits a reduced level of IgM and IgG antibody production or no IgM and IgG antibody production against the cells in a recipient subject. In some embodiments, the cells elicit a reduced level of cytotoxic T cell killing of the cells upon administration to a recipient subject.

### B. CD24

In some aspects, the present disclosure provides a stem cell (e.g., pluripotent stem cell or induced pluripotent stem cell) or population thereof that has been modified to express the tolerogenic factor (e.g., immunomodulatory polypeptide) CD24. In some aspects, the present disclosure provides a method for altering a stem cell genome to express CD24. In some embodiments, the stem cell expresses exogenous CD24. In some instances, the stem cell expresses an expression vector comprising a nucleotide sequence encoding a human CD24 polypeptide.

CD24 which is also referred to as a heat stable antigen or small-cell lung cancer cluster 4 antigen is a glycosylated glycosylphosphatidylinositol-anchored surface protein (Pirruccello et al., J Immunol, 1986, 136, 3779-3784; Chen et al., Glycobiology, 2017, 57, 800-806). It binds to Siglec-10 on innate immune cells. Recently it has been shown that CD24 via Siglec-10 acts as an innate immune checkpoint (Barkal et al., Nature, 2019, 572, 392-396).

In some embodiments, the cell outlined herein comprises a nucleotide sequence encoding a CD24 polypeptide has at least 90% sequence identity (e.g., 90%, 91%, 92%, 93%, 94%, 95%, 96%, 97%, 98%, 99%, or more) to a sequence selected from the group consisting of SEQ ID NO:28, SEQ ID NO:29, SEQ ID NO:30, and SEQ ID NO:31. In some embodiments, the cell outlined herein comprises a nucleotide sequence encoding a CD24 polypeptide has at least 95% sequence identity (e.g., 95%, 96%, 97%, 98%, 99%, or more) to a sequence selected from the group consisting of SEQ ID NO:28, SEQ ID NO:29, SEQ ID NO:30, and SEQ ID NO:31. In some embodiments, the cell outlined herein comprises a nucleotide sequence encoding a CD24 polypeptide having a sequence selected from the group consisting of SEQ ID NO:28, SEQ ID NO:29, SEQ ID NO:30, and SEQ ID NO:31.

In some embodiments, the cell outlined herein comprises a nucleotide sequence encoding a CD24 polypeptide has at least 90% sequence identity (e.g., 90%, 91%, 92%, 93%, 94%, 95%, 96%, 97%, 98%, 99%, or more) to an amino acid sequence set forth in NCBI Ref. Nos. NP_001278666.1, NP_001278667.1, NP_001278668.1, and NP_037362.1. In some instances, the CD24 polypeptide has at least 95% sequence identity (e.g., 95%, 96%, 97%, 98%, 99%, or more) to an amino acid sequence set forth in NCBI Ref. Nos. NP_001278666.1, NP_001278667.1, NP_001278668.1, and NP_037362.1. In some embodiments, the cell outlined herein comprises a nucleotide sequence encoding a CD24 polypeptide having an amino acid sequence set forth in NCBI Ref. Nos. NP_001278666.1, NP_001278667.1, NP_001278668.1, and NP_037362.1.

In some embodiments, the cell comprises a nucleotide sequence having at least 85% sequence identity (e.g., 85%, 86%, 87%, 88%, 89%, 90%, 91%, 92%, 93%, 94%, 95%, 96%, 97%, 98%, 99%, or more) to the sequence set forth in NCBI Ref. Nos. NM_00129737.1, NM_00129738.1, NM_001291739.1, and NM_013230.3. In some embodiments, the cell comprises a nucleotide sequence as set forth in NCBI Ref. Nos. NM_00129737.1, NM_00129738.1, NM_001291739.1, and NM_013230.3.

In another embodiment, CD24 protein expression is detected using a Western blot of cells lysates probed with antibodies to the CD24 protein. In another embodiment, reverse transcriptase polymerase chain reactions (RT-PCR) are used to confirm the presence of the exogenous CD24 mRNA.

### C. CIITA

In certain aspects, the inventions disclosed herein modulate (e.g., reduce or eliminate) the expression of MHC II genes by targeting and modulating (e.g., reducing or eliminating) Class II transactivator (CIITA) expression. In some aspects, the modulation occurs using a CRISPR/Cas system. CIITA is a member of the LR or nucleotide binding domain (NBD) leucine-rich repeat (LRR) family of proteins and regulates the transcription of MHC II by associating with the MHC enhanceosome.

In some embodiments, the target polynucleotide sequence of the present invention is a variant of CIITA. In some embodiments, the target polynucleotide sequence is a homolog of CIITA. In some embodiments, the target polynucleotide sequence is an ortholog of CIITA.

In some aspects, reduced or eliminated expression of CIITA reduces or eliminates expression of one or more of the following MHC class II are HLA-DP, HLA-DM, HLA-DOA, HLA-DOB, HLA-DQ, and HLA-DR.

In some embodiments, the cells outlined herein comprise a genetic modification targeting the CIITA gene. In some embodiments, the genetic modification targeting the CIITA gene by the rare-cutting endonuclease comprises a Cas protein or a polynucleotide encoding a Cas protein, and at least one guide ribonucleic acid sequence for specifically targeting the CIITA gene. In some embodiments, the at least one guide ribonucleic acid sequence for specifically targeting the CIITA gene is selected from the group consisting of SEQ ID NOS:5184-36352 of Appendix 1 or Table 12 of WO2016183041, the disclosure is herein incorporated by reference in its entirety.

Assays to test whether the CIITA gene has been inactivated are known and described herein. In one embodiment, the resulting genetic modification of the CIITA gene by PCR and the reduction of HLA-II expression can be assays by FACS analysis. In another embodiment, CIITA protein expression is detected using a Western blot of cells lysates probed with antibodies to the CIITA protein. In another embodiment, reverse transcriptase polymerase chain reactions (RT-PCR) are used to confirm the presence of the inactivating genetic modification.

### D. B2M

In certain embodiments, the inventions disclosed herein modulate (e.g., reduce or eliminate) the expression of MHC-I genes by targeting and modulating (e.g., reducing or eliminating) expression of the accessory chain B2M. In some aspects, the modulation occurs using a CRISPR/Cas system. By modulating (e.g., reducing or deleting) expression of B2M, surface trafficking of MHC-I molecules is blocked and such cells exhibit immune tolerance when engrafted into a recipient subject. In some embodiments, the cell is considered hypoimmunogenic, e.g., in a recipient subject or patient upon administration.

In some embodiments, the target polynucleotide sequence of the present invention is a variant of B2M. In some embodiments, the target polynucleotide sequence is a homolog of B2M. In some embodiments, the target polynucleotide sequence is an ortholog of B2M.

In some aspects, decreased or eliminated expression of B2M reduces or eliminates expression of one or more of the following MHC I molecules—HLA-A, HLA-B, and HLA-C.

In some embodiments, the hypoimmunogenic cells outlined herein comprise a genetic modification targeting the B2M gene. In some embodiments, the genetic modification targeting the B2M gene by the rare-cutting endonuclease comprises a Cas protein or a polynucleotide encoding a Cas protein, and at least one guide ribonucleic acid sequence for specifically targeting the B2M gene. In some embodiments, the at least one guide ribonucleic acid sequence for specifically targeting the B2M gene is selected from the group consisting of SEQ ID NOS:81240-85644 of Appendix 2 or Table 15 of WO2016/183041, the disclosure is herein incorporated by reference in its entirety.

Assays to test whether the B2M gene has been inactivated are known and described herein. In one embodiment, the resulting genetic modification of the B2M gene by PCR and the reduction of HLA-I expression can be assays by FACS analysis. In another embodiment, B2M protein expression is detected using a Western blot of cells lysates probed with antibodies to the B2M protein. In another embodiment, reverse transcriptase polymerase chain reactions (RT-PCR) are used to confirm the presence of the inactivating genetic modification.

### E. NLRC5

In certain aspects, the inventions disclosed herein modulate (e.g., reduce or eliminate) the expression of MHC-I genes by targeting and modulating (e.g., reducing or eliminating) expression of the NLR family, CARD domain containing 5/NOD27/CLR16.1 (NLRC5). In some aspects, the modulation occurs using a CRISPR/Cas system. NLRC5 is a critical regulator of MHC-I-mediated immune responses and, similar to CIITA, NLRC5 is highly inducible by IFN-γ and can translocate into the nucleus. NLRC5 activates the promoters of MHC-I genes and induces the transcription of MHC-I as well as related genes involved in MHC-I antigen presentation.

In some embodiments, the target polynucleotide sequence of the present invention is a variant of NLRC5. In some embodiments, the target polynucleotide sequence is a homolog of NLRC5. In some embodiments, the target polynucleotide sequence is an ortholog of NLRC5.

In some aspects, decreased or eliminated expression of NLRC5 reduces or eliminates expression of one or more of the following MHC I molecules— HLA-A, HLA-B, and HLA-C.

In some embodiments, the cells outlined herein comprise a genetic modification targeting the NLRC5 gene. In some embodiments, the genetic modification targeting the NLRC5 gene by the rare-cutting endonuclease comprises a Cas protein or a polynucleotide encoding a Cas protein, and at least one guide ribonucleic acid sequence for specifically targeting the NLRC5 gene. In some embodiments, the at least one guide ribonucleic acid sequence for specifically targeting the NLRC5 gene is selected from the group consisting of SEQ ID NOS:36353-81239 of Appendix 3 or Table 14 of WO2016183041, the disclosure is herein incorporated by reference in its entirety.

Assays to test whether the NLRC5 gene has been inactivated are known and described herein. In one embodiment, the resulting genetic modification of the NLRC5 gene by PCR and the reduction of HLA-I expression can be assays by FACS analysis. In another embodiment, NLRC5 protein expression is detected using a Western blot of cells lysates probed with antibodies to the NLRC5 protein. In another embodiment, reverse transcriptase polymerase chain reactions (RT-PCR) are used to confirm the presence of the inactivating genetic modification.

### F. CD47

In some embodiments, the cell comprises an exogenous CD24 polypeptide and an exogenous CD47 polypeptide. In some embodiments, the pluripotent cell or differentiated cell generated from the pluripotent cell comprises an exogenous CD24 polypeptide and an exogenous CD47 polypeptide.

In some aspects, the present disclosure provides a cell or population thereof that has been modified to express the tolerogenic factor (e.g., immunomodulatory polypeptide) CD47. In some aspects, the present disclosure provides a method for altering a cell genome to express CD47. In some embodiments, the stem cell expresses exogenous CD47 polynucleotides and/or polypeptides. In some instances, the cell expresses an expression vector comprising a nucleotide sequence encoding a human CD47 polypeptide.

CD47 is a leukocyte surface antigen and has a role in cell adhesion and modulation of integrins. It is expressed on the surface of a cell and signals to circulating macrophages not to eat the cell.

In some embodiments, the cell outlined herein comprises a nucleotide sequence encoding a CD47 polypeptide has at least 95% sequence identity (e.g., 95%, 96%, 97%, 98%, 99%, or more) to an amino acid sequence as set forth in NCBI Ref. Sequence Nos. NP_001768.1 and NP_942088.1, and SEQ ID NOS:32 and 33. In some embodiments, the cell outlined herein comprises a nucleotide sequence encoding a CD47 polypeptide having an amino acid sequence as set forth in NCBI Ref. Sequence Nos. NP_001768.1 and NP_942088.1, and SEQ ID NOS:32 and 33. In some embodiments, the cell comprises a nucleotide sequence for CD47 having at least 85% sequence identity (e.g., 85%, 86%, 87%, 88%, 89%, 90%, 91%, 92%, 93%, 94%, 95%, 96%, 97%, 98%, 99%, or more) to the sequence set forth in NCBI Ref. Nos. NM_001777.3 and NM_198793.2. In some embodiments, the cell comprises a nucleotide sequence for CD47 as set forth in NCBI Ref. Sequence Nos. NM_001777.3 and NM_198793.2.

In some embodiments, the cell comprises a CD47 polypeptide having at least 95% sequence identity (e.g., 95%, 96%, 97%, 98%, 99%, or more) to an amino acid sequence as set forth in NCBI Ref. Sequence Nos. NP_001768.1 and NP_942088.1, and SEQ ID NOS:32 and 33. In some embodiments, the cell outlined herein comprises a CD47 polypeptide having an amino acid sequence as set forth in NCBI Ref. Sequence Nos. NP_001768.1 and NP_942088.1, and SEQ ID NOS:32 and 33.

In some embodiments, a gene editing system such as a CRISPR/Cas system is used to facilitate the insertion of tolerogenic factors, such as the insertion of tolerogenic factors into a safe harbor locus, such as the AAVS1 locus. In some cases, the polynucleotide sequence encoding CD47 is inserted into a safe harbor locus, such as but not limited to, an AAVS1, CCR5, CLYBL, ROSA26, or SHS231 locus.

In another embodiment, CD47 protein expression is detected using a Western blot of cell lysates probed with antibodies against the CD47 protein. In another embodiment, reverse transcriptase polymerase chain reactions (RT-PCR) are used to confirm the presence of the exogenous CD47 mRNA.

### G. DUX4

In some aspects, the present disclosure provides a cell (e.g., a stem cell, pluripotent stem cell, induced pluripotent stem cell, differentiated cell derived or produced from such a stem cell, hematopoietic stem cell, primary T cell, chimeric antigen receptor (CAR) T cell, and any progeny thereof) or population thereof comprising a genome modified to increase expression of a tolerogenic or immunosuppressive factor such as DUX4. In some aspects, the present disclosure provides a method for altering a cell's genome to provide increased expression of DUX4. In one aspect, the disclosure provides a cell or population thereof comprising exogenously expressed DUX4 proteins. In some aspects, increased expression of DUX4 suppresses, reduces or eliminates expression of one or more of the following MHC I molecules—HLA-A, HLA-B, and HLA-C.

In some embodiments, the cell comprises an exogenous CD24 polypeptide and an exogenous DUX4 polypeptide. In some embodiments, the pluripotent cell or differentiated cell generated from the pluripotent cell comprises an exogenous CD24 polypeptide and an exogenous DUX4 polypeptide.

DUX4 is a transcription factor that is active in embryonic tissues and induced pluripotent stem cells, and is silent in normal, healthy somatic tissues (Feng et al., 2015, ELife4; De Iaco et al., 2017, Nat Genet, 49, 941-945; Hendrickson et al., 2017, Nat Genet, 49, 925-934; Snider et al., 2010, PLoS Genet, e1001181; Whiddon et al., 2017, Nat Genet). DUX4 expression acts to block IFN-gamma mediated induction of major histocompatibility complex (MHC) class I gene expression (e.g., expression of B2M, HLA-A, HLA-B, and HLA-C). DUX4 expression has been implicated in suppressed antigen presentation by MHC class I (Chew et al., Developmental Cell, 2019, 50, 1-14). DUX4 functions as a transcription factor in the cleavage-stage gene expression (transcriptional) program. Its target genes include, but are not limited to, coding genes, noncoding genes, and repetitive elements.

There are at least two isoforms of DUX4, with the longest isoform comprising the DUX4 C-terminal transcription activation domain. The isoforms are produced by alternative splicing. See, e.g., Geng et al., 2012, Dev Cell, 22, 38-51; Snider et al., 2010, PLoS Genet, e1001181. Active isoforms for DUX4 comprise its N-terminal DNA-binding domains and its C-terminal activation domain. See, e.g., Choi et al., 2016, Nucleic Acid Res, 44, 5161-5173.

It has been shown that reducing the number of CpG motifs of DUX4 decreases silencing of a DUX4 transgene (Jagannathan et al., Human Molecular Genetics, 2016, 25(20):4419-4431). SEQ ID NO:1 represents a codon altered sequence of DUX4 comprising one or more base substitutions to reduce the total number of CpG sites while preserving the DUX4 protein sequence. The nucleic acid sequence is commercially available from Addgene, Catalog No. 99281.

In certain aspects, at least one or more polynucleotides may be utilized to facilitate the insertion of DUX4 into a cell, e.g., a stem cell, induced pluripotent stem cell, differentiated cell, hematopoietic stem cell, primary T cell or CAR-T cell.

In some embodiments, a gene editing system such as the CRISPR/Cas system is used to facilitate the insertion of tolerogenic factors, such as the insertion of tolerogenic factors into a safe harbor locus, such as the AAVS1 locus. In some cases, the polynucleotide sequence encoding DUX4 is inserted into a safe harbor locus, such as but not limited to, an AAVS1, CCR5, CLYBL, ROSA26, or SHS231 locus.

In some cases, the polynucleotide sequence encoding DUX4 is SEQ ID NO:1. In some embodiments, the polynucleotide sequence encoding DUX4 is a nucleotide sequence encoding a polypeptide sequence having at least 90% (e.g., 90%, 91%, 92%, 93%, 94%, 95%, 96%, 97%, 98%, 99%, or more) sequence identity to a sequence selected from the group consisting of SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, and SEQ ID NO:25. In some embodiments, the polynucleotide sequence encoding DUX4 is a nucleotide sequence encoding a polypeptide sequence is selected from the group consisting of SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, and SEQ ID NO:25.

In other embodiments, expression of tolerogenic factors is facilitated using an expression vector. In some embodiments, the expression vector comprises a polynucleotide sequence encoding DUX4 is a codon altered sequence comprising one or more base substitutions to reduce the total number of CpG sites while preserving the DUX4 protein sequence. In some cases, the codon altered sequence of DUX4 is SEQ ID NO:1. In other embodiments, the expression vector comprises a polynucleotide sequence encoding DUX4 is SEQ ID NO:1. In some embodiments, the expression vector comprises a polynucleotide sequence encoding DUX4 is a nucleotide sequence encoding a polypeptide sequence having at least 90% (e.g., 90%, 91%, 92%, 93%, 94%, 95%, 96%, 97%, 98%, 99%, or more) sequence identity to a sequence selected from the group consisting of SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, and SEQ ID NO:25. In some embodiments, the expression vector comprises a polynucleotide sequence encoding DUX4 is a nucleotide sequence encoding a polypeptide sequence is selected from the group consisting of SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, and SEQ ID NO:25.

An increase of DUX4 expression can be assayed using known techniques, such as Western blots, ELISA assays, FACS assays, immunoassays, and the like.

### H. Additional Tolerogenic Factors

In certain embodiments, one or more tolerogenic factors can be inserted or reinserted into genome-edited cells to create immune-privileged universal donor cells, such as universal donor stem cells, universal donor T cells, or universal donor cells. In certain embodiments, the hypoimmunogenic cells disclosed herein have been further modified to express one or more tolerogenic factors. Exemplary tolerogenic factors include, without limitation, one or more of CD24, CD47, DUX4, CD27, CD35, CD46, CD55, CD59, CD200, HLA-C, HLA-E, HLA-E heavy chain, HLA-G, PD-L1, IDO1, CTLA4-Ig, C1-Inhibitor, IL-10, IL-35, FASL, CCL21, Mfge8, and Serpinb9. In some embodiments, the tolerogenic factors are selected from the group consisting of CD47, DUX4, CD27, CD35, CD46, CD55, CD59, CD200, HLA-C, HLA-E, HLA-E heavy chain, HLA-G, PD-L1, IDO1, CTLA4-Ig, C1-Inhibitor, IL-10, IL-35, FASL, CCL21, Mfge8, and Serpinb9.

In some embodiments, a gene editing system such as the CRISPR/Cas system is used to facilitate the insertion of tolerogenic factors, such as the insertion of tolerogenic factors into a safe harbor locus, such as the AAVS1 locus. In some cases, the polynucleotide sequence encoding any tolerogenic factor described herein is inserted into a safe harbor locus, such as but not limited to, an AAVS1, CCR5, CLYBL, ROSA26, or SHS231 locus.

In some aspects, the present disclosure provides a cell (e.g., a stem cell, pluripotent stem cell, induced pluripotent stem cell, differentiated cell derived or produced from such a stem cell, hematopoietic stem cell, primary T cell, chimeric antigen receptor (CAR) T cell, and any progeny thereof) or population thereof comprising a genome in which the cell genome has been modified to express CD47. In some aspects, the present disclosure provides a method for altering a cell genome to express CD47. In certain aspects at least one ribonucleic acid or at least one pair of ribonucleic acids may be utilized to facilitate the insertion of CD47 into a cell line. In certain embodiments, the at least one ribonucleic acid or the at least one pair of ribonucleic acids is selected from the group consisting of SEQ ID NOS:200784-231885 of Table 29 of WO2016183041, which is herein incorporated by reference.

In some aspects, the present disclosure provides a cell (e.g., a stem cell, pluripotent stem cell, induced pluripotent stem cell, differentiated cell derived or produced from such a stem cell, hematopoietic stem cell, primary T cell, chimeric antigen receptor (CAR) T cell, and any progeny thereof) or population thereof comprising a genome in which the cell genome has been modified to express HLA-C. In some aspects, the present disclosure provides a method for altering a cell genome to express HLA-C. In certain aspects at least one ribonucleic acid or at least one pair of ribonucleic acids may be utilized to facilitate the insertion of HLA-C into a cell line. In certain embodiments, the at least one ribonucleic acid or the at least one pair of ribonucleic acids is selected from the group consisting of SEQ ID NOS:3278-5183 of Table 10 of WO2016183041, which is herein incorporated by reference.

In some aspects, the present disclosure provides a cell (e.g., a stem cell, pluripotent stem cell, induced pluripotent stem cell, differentiated cell derived or produced from such a stem cell, hematopoietic stem cell, primary T cell, chimeric antigen receptor (CAR) T cell, and any progeny thereof) or population thereof comprising a genome in which the cell genome has been modified to express HLA-E. In some aspects, the present disclosure provides a method for altering a cell genome to express HLA-E. In certain aspects at least one ribonucleic acid or at least one pair of ribonucleic acids may be utilized to facilitate the insertion of HLA-E into a cell line. In certain embodiments, the at least one ribonucleic acid or the at least one pair of ribonucleic acids is selected from the group consisting of SEQ ID NOS:189859-193183 of Table 19 of WO2016183041, which is herein incorporated by reference.

In some aspects, the present disclosure provides a cell (e.g., a stem cell, pluripotent stem cell, induced pluripotent stem cell, differentiated cell derived or produced from such a stem cell, hematopoietic stem cell, primary T cell, chimeric antigen receptor (CAR) T cell, and any progeny thereof) or population thereof comprising a genome in which the cell genome has been modified to express HLA-F. In some aspects, the present disclosure provides a method for altering a cell genome to express HLA-F. In certain aspects at least one ribonucleic acid or at least one pair of ribonucleic acids may be utilized to facilitate the insertion of HLA-F into a cell line. In certain embodiments, the at least one ribonucleic acid or the at least one pair of ribonucleic acids is selected from the group consisting of SEQ ID NOS: 688808-399754 of Table 45 of WO2016183041, which is herein incorporated by reference.

In some aspects, the present disclosure provides a cell (e.g., a stem cell, pluripotent stem cell, induced pluripotent stem cell, differentiated cell derived or produced from such a stem cell, hematopoietic stem cell, primary T cell, chimeric antigen receptor (CAR) T cell, and any progeny thereof) or population thereof comprising a genome in which the cell genome has been modified to express HLA-G. In some aspects, the present disclosure provides a method for altering a cell genome to express HLA-G. In certain aspects at least one ribonucleic acid or at least one pair of ribonucleic acids may be utilized to facilitate the insertion of HLA-G into a stem cell line. In certain embodiments, the at least one ribonucleic acid or the at least one pair of ribonucleic acids is selected from the group consisting of SEQ ID NOS:188372-189858 of Table 18 of WO2016183041, which is herein incorporated by reference.

In some aspects, the present disclosure provides a cell (e.g., a stem cell, pluripotent stem cell, induced pluripotent stem cell, differentiated cell derived or produced from such a stem cell, hematopoietic stem cell, primary T cell, chimeric antigen receptor (CAR) T cell, and any progeny thereof) or population thereof comprising a genome in which the cell genome has been modified to express PD-L1. In some aspects, the present disclosure provides a method for altering a cell genome to express PD-L1. In certain aspects at least one ribonucleic acid or at least one pair of ribonucleic acids may be utilized to facilitate the insertion of PD-L1 into a stem cell line. In certain embodiments, the at least one ribonucleic acid or the at least one pair of ribonucleic acids is selected from the group consisting of SEQ ID NOS:193184-200783 of Table 21 of WO2016183041, which is herein incorporated by reference.

In some aspects, the present disclosure provides a cell (e.g., a stem cell, pluripotent stem cell, induced pluripotent stem cell, differentiated cell derived or produced from such a stem cell, hematopoietic stem cell, primary T cell, chimeric antigen receptor (CAR) T cell, and any progeny thereof) or population thereof comprising a genome in which the cell genome has been modified to express CTLA4-Ig. In some aspects, the present disclosure provides a method for altering a cell genome to express CTLA4-Ig. In certain aspects at least one ribonucleic acid or at least one pair of ribonucleic acids may be utilized to facilitate the insertion of CTLA4-Ig into a stem cell line. In certain embodiments, the at least one ribonucleic acid or the at least one pair of ribonucleic acids is selected from any one disclosed in WO2016183041, including the sequence listing.

In some aspects, the present disclosure provides a cell (e.g., a stem cell, pluripotent stem cell, induced pluripotent stem cell, differentiated cell derived or produced from such a stem cell, hematopoietic stem cell, primary T cell, chimeric antigen receptor (CAR) T cell, and any progeny thereof) or population thereof comprising a genome in which the cell genome has been modified to express CI-inhibitor. In some aspects, the present disclosure provides a method for altering a cell genome to express CI-inhibitor. In certain aspects at least one ribonucleic acid or at least one pair of ribonucleic acids may be utilized to facilitate the insertion of CI-inhibitor into a stem cell line. In certain embodiments, the at least one ribonucleic acid or the at least one pair of ribonucleic acids is selected from any one disclosed in WO2016183041, including the sequence listing.

In some aspects, the present disclosure provides a cell (e.g., a stem cell, pluripotent stem cell, induced pluripotent stem cell, differentiated cell derived or produced from such a stem cell, hematopoietic stem cell, primary T cell, chimeric antigen receptor (CAR) T cell, and any progeny thereof) or population thereof comprising a genome in which the cell genome has been modified to express IL-35. In some aspects, the present disclosure provides a method for altering a cell genome to express IL-35. In certain aspects at least one ribonucleic acid or at least one pair of ribonucleic acids may be utilized to facilitate the insertion of IL-35 into a stem cell line. In certain embodiments, the at least one ribonucleic acid or the at least one pair of ribonucleic acids is selected from any one disclosed in WO2016183041, including the sequence listing.

In some embodiments, the tolerogenic factors are expressed in a cell using an expression vector. For example, the expression vector for expressing CD47 in a cell comprises a polynucleotide sequence encoding a CD47 polypeptide. In some embodiments, the CD47 polypeptide comprises the amino acid sequence of SEQ ID NO:32 or SEQ ID NO:33. The expression vector can be an inducible expression vector. The expression vector can be a viral vector, such as but not limited to, a lentiviral vector.

In some embodiments, the present disclosure provides a cell (e.g., a stem cell, pluripotent stem cell, induced pluripotent stem cell, differentiated cell derived or produced from such a stem cell, hematopoietic stem cell, primary T cell, chimeric antigen receptor (CAR) T cell, and any progeny thereof) or population thereof comprising a genome in which the cell genome has been modified to express any one of the polypeptides selected from the group consisting of HLA-A, HLA-B, HLA-C, RFX-ANK, CIITA, NFY-A, NLRC5, B2M, RFX5, RFX-AP, HLA-G, HLA-E, NFY-B, PD-L1, NFY-C, IRF1, TAP1, GITR, 4-1BB, CD28, B7-1, CD47, B7-2, OX40, CD27, HVEM, SLAM, CD226, ICOS, LAG3, TIGIT, TIM3, CD160, BTLA, CD244, LFA-1, ST2, HLA-F, CD30, B7-H3, VISTA, TLT, PD-L2, CD58, CD2, HELIOS, and IDO1. In some aspects, the present disclosure provides a method for altering a cell genome to express any one of the polypeptides selected from the group consisting of HLA-A, HLA-B, HLA-C, RFX-ANK, CIITA, NFY-A, NLRC5, B2M, RFX5, RFX-AP, HLA-G, HLA-E, NFY-B, PD-L1, NFY-C, IRF1, TAP1, GITR, 4-1BB, CD28, B7-1, CD47, B7-2, OX40, CD27, HVEM, SLAM, CD226, ICOS, LAG3, TIGIT, TIM3, CD160, BTLA, CD244, LFA-1, ST2, HLA-F, CD30, B7-H3, VISTA, TLT, PD-L2, CD58, CD2, HELIOS, and IDO1. In certain aspects at least one ribonucleic acid or at least one pair of ribonucleic acids may be utilized to facilitate the insertion of the selected polypeptide into a stem cell line. In certain embodiments, the at least one ribonucleic acid or the at least one pair of ribonucleic acids is selected from any one disclosed in Appendices 1-47 and the sequence listing of WO2016183041, the disclosure is incorporated herein by references.

### I. Exemplary Embodiments

In some embodiments, the cells and populations thereof exhibit increased expression of CD24 and reduced expression of one or more molecules of the MHC class I complex. In some embodiments, the cells and populations thereof exhibit increased expression of CD24 and reduced expression of one or more molecules of the MHC class II complex. In some embodiments, the cells and populations thereof exhibit increased expression of CD24 and reduced expression of one or more molecules of the MHC class II and MHC class II complexes.

In some embodiments, the cells and populations thereof exhibit increased expression of CD24 and reduced expression of B2M. In some embodiments, the cells and populations thereof exhibit increased expression of CD24 and reduced expression of CIITA. In some embodiments, the cells and populations thereof exhibit increased expression of CD24 and reduced expression of NLRC5. In some embodiments, the cells and populations thereof exhibit increased expression of CD24 and reduced expression of one or more molecules of B2M and CIITA. In some embodiments, the cells and populations thereof exhibit increased expression of CD24 and reduced expression of one or more molecules of B2M and NLRC5. In some embodiments, the cells and populations thereof exhibit increased expression of CD24 and reduced expression of one or more molecules of CIITA and NLRC5. In some embodiments, the cells and populations thereof exhibit increased expression of CD24 and reduced expression of one or more molecules of B2M, CIITA and NLRC5. Any of the cells described herein can also exhibit increased expression of one or more factors selected from the group including, but not limited to, CD47, CD27, CD35, CD46, CD55, CD59, CD200, HLA-C, HLA-E, HLA-E heavy chain, HLA-G, PD-L1, IDO1, CTLA4-Ig, C1-Inhibitor, IL-10, IL-35, FASL, CCL21, Mfge8, and Serpinb9.

In some embodiments, the cells and populations thereof exhibit increased expression of CD24 and CD47 and reduced expression of one or more molecules of the MHC class I complex. In some embodiments, the cells and populations thereof exhibit increased expression of CD24 and CD47 and reduced expression of one or more molecules of the MHC class II complex. In some embodiments, the cells and populations thereof exhibit increased expression of CD24 and CD47 and reduced expression of one or more molecules of the MHC class II and MHC class II complexes. In some embodiments, the cells and populations thereof exhibit increased expression of CD24 and CD47 and reduced expression of B2M. In some embodiments, the cells and populations thereof exhibit increased expression of CD24 and CD47 and reduced expression of CIITA. In some embodiments, the cells and populations thereof exhibit increased expression of CD24 and CD47 and reduced expression of NLRC5. In some embodiments, the cells and populations thereof exhibit increased expression of CD24 and CD47 and reduced expression of one or more molecules of B2M and CIITA. In some embodiments, the cells and populations thereof exhibit increased expression of CD24 and CD47 and reduced expression of one or more molecules of B2M and NLRC5. In some embodiments, the cells and populations thereof exhibit increased expression of CD24 and CD47 and reduced expression of one or more molecules of CIITA and NLRC5. In some embodiments, the cells and populations thereof exhibit increased expression of CD24 and CD47 and reduced expression of one or more molecules of B2M, CIITA and NLRC5. Any of the cells described herein can also exhibit increased expression of one or more selected from the group including, but not limited to, CD27, CD35, CD46, CD55, CD59, CD200, HLA-C, HLA-E, HLA-E heavy chain, HLA-G, PD-L1, IDO1, CTLA4-Ig, C1-Inhibitor, IL-10, IL-35, FASL, CCL21, Mfge8, and Serpinb9.

One skilled in the art will appreciate that levels of expression such as increased or reduced expression of a gene, protein or molecule in an engineered or modified can be referenced or compared to a comparable unengineered or unmodified cell. In some embodiments, an engineered stem cell having increased expression of CD24 refers to a modified stem cell having a higher level of CD24 protein compared to an unmodified stem cell.

### J. Methods of Genetic Modifications

In some embodiments, the rare-cutting endonuclease is introduced into a cell containing the target polynucleotide sequence in the form of a nucleic acid encoding a rare-cutting endonuclease. The process of introducing the nucleic acids into cells can be achieved by any suitable technique. Suitable techniques include calcium phosphate or lipid-mediated transfection, electroporation, and transduction or infection using a viral vector. In some embodiments, the nucleic acid comprises DNA. In some embodiments, the nucleic acid comprises a modified DNA, as described herein. In some embodiments, the nucleic acid comprises mRNA. In some embodiments, the nucleic acid comprises a modified mRNA, as described herein (e.g., a synthetic, modified mRNA).

The present invention contemplates altering target polynucleotide sequences in any manner which is available to the skilled artisan utilizing a CRISPR/Cas system of the present invention. Any CRISPR/Cas system that is capable of altering a target polynucleotide sequence in a cell can be used. Such CRISPR-Cas systems can employ a variety of Cas proteins (Haft et al. PLoS Comput Biol. 2005; 1(6)e60). The molecular machinery of such Cas proteins that allows the CRISPR/Cas system to alter target polynucleotide sequences in cells include RNA binding proteins, endo- and exo-nucleases, helicases, and polymerases. In some embodiments, the CRISPR/Cas system is a CRISPR type I system. In some embodiments, the CRISPR/Cas system is a CRISPR type II system. In some embodiments, the CRISPR/Cas system is a CRISPR type V system.

The CRISPR/Cas systems of the present invention can be used to alter any target polynucleotide sequence in a cell. Those skilled in the art will readily appreciate that desirable target polynucleotide sequences to be altered in any particular cell may correspond to any genomic sequence for which expression of the genomic sequence is associated with a disorder or otherwise facilitates entry of a pathogen into the cell. For example, a desirable target polynucleotide sequence to alter in a cell may be a polynucleotide sequence corresponding to a genomic sequence which contains a disease associated single polynucleotide polymorphism. In such example, the CRISPR/Cas systems of the present invention can be used to correct the disease associated SNP in a cell by replacing it with a wild-type allele. As another example, a polynucleotide sequence of a target gene which is responsible for entry or proliferation of a pathogen into a cell may be a suitable target for deletion or insertion to disrupt the function of the target gene to prevent the pathogen from entering the cell or proliferating inside the cell.

In some embodiments, the target polynucleotide sequence is a genomic sequence. In some embodiments, the target polynucleotide sequence is a human genomic sequence. In some embodiments, the target polynucleotide sequence is a mammalian genomic sequence. In some embodiments, the target polynucleotide sequence is a vertebrate genomic sequence.

In some embodiments, a CRISPR/Cas system of the present invention includes a Cas protein and at least one to two ribonucleic acids that are capable of directing the Cas protein to and hybridizing to a target motif of a target polynucleotide sequence. As used herein, “protein” and “polypeptide” are used interchangeably to refer to a series of amino acid residues joined by peptide bonds (i.e., a polymer of amino acids) and include modified amino acids (e.g., phosphorylated, glycated, glycosylated, etc.) and amino acid analogs. Exemplary polypeptides or proteins include gene products, naturally occurring proteins, homologs, paralogs, fragments and other equivalents, variants, and analogs of the above.

In some embodiments, a Cas protein comprises one or more amino acid substitutions or modifications. In some embodiments, the one or more amino acid substitutions comprises a conservative amino acid substitution. In some instances, substitutions and/or modifications can prevent or reduce proteolytic degradation and/or extend the half-life of the polypeptide in a cell. In some embodiments, the Cas protein can comprise a peptide bond replacement (e.g., urea, thiourea, carbamate, sulfonyl urea, etc.). In some embodiments, the Cas protein can comprise a naturally occurring amino acid. In some embodiments, the Cas protein can comprise an alternative amino acid (e.g., D-amino acids, beta-amino acids, homocysteine, phosphoserine, etc.). In some embodiments, a Cas protein can comprise a modification to include a moiety (e.g., PEGylation, glycosylation, lipidation, acetylation, end-capping, etc.).

In some embodiments, a Cas protein comprises a core Cas protein. Exemplary Cas core proteins include, but are not limited to Cas1, Cas2, Cas3, Cas4, Cas5, Cas6, Cas7, Cas8 and Cas9. In some embodiments, a Cas protein comprises a Cas protein of an E. coli subtype (also known as CASS2). Exemplary Cas proteins of the E. Coli subtype include, but are not limited to Cse1, Cse2, Cse3, Cse4, and Cas5e. In some embodiments, a Cas protein comprises a Cas protein of the Ypest subtype (also known as CASS3). Exemplary Cas proteins of the Ypest subtype include, but are not limited to Csy1, Csy2, Csy3, and Csy4. In some embodiments, a Cas protein comprises a Cas protein of the Nmeni subtype (also known as CASS4). Exemplary Cas proteins of the Nmeni subtype include, but are not limited to Csn1 and Csn2. In some embodiments, a Cas protein comprises a Cas protein of the Dvulg subtype (also known as CASS1). Exemplary Cas proteins of the Dvulg subtype include Csd1, Csd2, and Cas5d. In some embodiments, a Cas protein comprises a Cas protein of the Tneap subtype (also known as CASS7). Exemplary Cas proteins of the Tneap subtype include, but are not limited to, Cst1, Cst2, Cas5t. In some embodiments, a Cas protein comprises a Cas protein of the Hmari subtype. Exemplary Cas proteins of the Hmari subtype include, but are not limited to Csh1, Csh2, and Cas5h. In some embodiments, a Cas protein comprises a Cas protein of the Apern subtype (also known as CASS5). Exemplary Cas proteins of the Apern subtype include, but are not limited to Csa1, Csa2, Csa3, Csa4, Csa5, and Cas5a. In some embodiments, a Cas protein comprises a Cas protein of the Mtube subtype (also known as CASS6). Exemplary Cas proteins of the Mtube subtype include, but are not limited to Csm1, Csm2, Csm3, Csm4, and Csm5. In some embodiments, a Cas protein comprises a RAMP module Cas protein. Exemplary RAMP module Cas proteins include, but are not limited to, Cmr1, Cmr2, Cmr3, Cmr4, Cmr5, and Cmr6. See, e.g., Klompe et al., Nature 571, 219-225 (2019); Strecker et al., Science 365, 48-53 (2019).

In some embodiments, a Cas protein comprises any one of the Cas proteins described herein or a functional portion thereof. As used herein, “functional portion” refers to a portion of a peptide which retains its ability to complex with at least one ribonucleic acid (e.g., guide RNA (gRNA)) and cleave a target polynucleotide sequence. In some embodiments, the functional portion comprises a combination of operably linked Cas9 protein functional domains selected from the group consisting of a DNA binding domain, at least one RNA binding domain, a helicase domain, and an endonuclease domain. In some embodiments, the functional portion comprises a combination of operably linked Cas12a (also known as Cpf1) protein functional domains selected from the group consisting of a DNA binding domain, at least one RNA binding domain, a helicase domain, and an endonuclease domain. In some embodiments, the functional domains form a complex. In some embodiments, a functional portion of the Cas9 protein comprises a functional portion of a RuvC-like domain. In some embodiments, a functional portion of the Cas9 protein comprises a functional portion of the HNH nuclease domain. In some embodiments, a functional portion of the Cas12a protein comprises a functional portion of a RuvC-like domain.

In some embodiments, exogenous Cas protein can be introduced into the cell in polypeptide form. In certain embodiments, Cas proteins can be conjugated to or fused to a cell-penetrating polypeptide or cell-penetrating peptide. As used herein, “cell-penetrating polypeptide” and “cell-penetrating peptide” refers to a polypeptide or peptide, respectively, which facilitates the uptake of molecule into a cell. The cell-penetrating polypeptides can contain a detectable label.

In certain embodiments, Cas proteins can be conjugated to or fused to a charged protein (e.g., that carries a positive, negative or overall neutral electric charge). Such linkage may be covalent. In some embodiments, the Cas protein can be fused to a superpositively charged GFP to significantly increase the ability of the Cas protein to penetrate a cell (Cronican et al. ACS Chem Biol. 2010; 5(8):747-52). In certain embodiments, the Cas protein can be fused to a protein transduction domain (PTD) to facilitate its entry into a cell. Exemplary PTDs include Tat, oligoarginine, and penetratin. In some embodiments, the Cas9 protein comprises a Cas9 polypeptide fused to a cell-penetrating peptide. In some embodiments, the Cas9 protein comprises a Cas9 polypeptide fused to a PTD. In some embodiments, the Cas9 protein comprises a Cas9 polypeptide fused to a tat domain. In some embodiments, the Cas9 protein comprises a Cas9 polypeptide fused to an oligoarginine domain. In some embodiments, the Cas9 protein comprises a Cas9 polypeptide fused to a penetratin domain. In some embodiments, the Cas9 protein comprises a Cas9 polypeptide fused to a superpositively charged GFP. In some embodiments, the Cas12a protein comprises a Cas12a polypeptide fused to a cell-penetrating peptide. In some embodiments, the Cas12a protein comprises a Cas12a polypeptide fused to a PTD. In some embodiments, the Cas12a protein comprises a Cas12a polypeptide fused to a tat domain. In some embodiments, the Cas12a protein comprises a Cas12a polypeptide fused to an oligoarginine domain. In some embodiments, the Cas12a protein comprises a Cas12a polypeptide fused to a penetratin domain. In some embodiments, the Cas12a protein comprises a Cas12a polypeptide fused to a superpositively charged GFP.

In some embodiments, the Cas protein can be introduced into a cell containing the target polynucleotide sequence in the form of a nucleic acid encoding the Cas protein. The process of introducing the nucleic acids into cells can be achieved by any suitable technique. Suitable techniques include calcium phosphate or lipid-mediated transfection, electroporation, and transduction or infection using a viral vector. In some embodiments, the nucleic acid comprises DNA. In some embodiments, the nucleic acid comprises a modified DNA, as described herein. In some embodiments, the nucleic acid comprises mRNA. In some embodiments, the nucleic acid comprises a modified mRNA, as described herein (e.g., a synthetic, modified mRNA).

In some embodiments, the Cas protein is complexed with one to two ribonucleic acids. In some embodiments, the Cas protein is complexed with two ribonucleic acids. In some embodiments, the Cas protein is complexed with one ribonucleic acid. In some embodiments, the Cas protein is encoded by a modified nucleic acid, as described herein (e.g., a synthetic, modified mRNA).

The methods of the present invention contemplate the use of any ribonucleic acid that is capable of directing a Cas protein to and hybridizing to a target motif of a target polynucleotide sequence. In some embodiments, at least one of the ribonucleic acids comprises tracrRNA. In some embodiments, at least one of the ribonucleic acids comprises CRISPR RNA (crRNA). In some embodiments, a single ribonucleic acid comprises a guide RNA that directs the Cas protein to and hybridizes to a target motif of the target polynucleotide sequence in a cell. In some embodiments, at least one of the ribonucleic acids comprises a guide RNA that directs the Cas protein to and hybridizes to a target motif of the target polynucleotide sequence in a cell. In some embodiments, both of the one to two ribonucleic acids comprise a guide RNA that directs the Cas protein to and hybridizes to a target motif of the target polynucleotide sequence in a cell. The ribonucleic acids of the present invention can be selected to hybridize to a variety of different target motifs, depending on the particular CRISPR/Cas system employed, and the sequence of the target polynucleotide, as will be appreciated by those skilled in the art. The one to two ribonucleic acids can also be selected to minimize hybridization with nucleic acid sequences other than the target polynucleotide sequence. In some embodiments, the one to two ribonucleic acids hybridize to a target motif that contains at least two mismatches when compared with all other genomic nucleotide sequences in the cell. In some embodiments, the one to two ribonucleic acids hybridize to a target motif that contains at least one mismatch when compared with all other genomic nucleotide sequences in the cell. In some embodiments, the one to two ribonucleic acids are designed to hybridize to a target motif immediately adjacent to a deoxyribonucleic acid motif recognized by the Cas protein. In some embodiments, each of the one to two ribonucleic acids are designed to hybridize to target motifs immediately adjacent to deoxyribonucleic acid motifs recognized by the Cas protein which flank a mutant allele located between the target motifs.

In some embodiments, each of the one to two ribonucleic acids comprises guide RNAs that directs the Cas protein to and hybridizes to a target motif of the target polynucleotide sequence in a cell.

In some embodiments, one or two ribonucleic acids (e.g., guide RNAs) are complementary to and/or hybridize to sequences on the same strand of a target polynucleotide sequence. In some embodiments, one or two ribonucleic acids (e.g., guide RNAs) are complementary to and/or hybridize to sequences on the opposite strands of a target polynucleotide sequence. In some embodiments, the one or two ribonucleic acids (e.g., guide RNAs) are not complementary to and/or do not hybridize to sequences on the opposite strands of a target polynucleotide sequence. In some embodiments, the one or two ribonucleic acids (e.g., guide RNAs) are complementary to and/or hybridize to overlapping target motifs of a target polynucleotide sequence. In some embodiments, the one or two ribonucleic acids (e.g., guide RNAs) are complementary to and/or hybridize to offset target motifs of a target polynucleotide sequence.

In some embodiments, nucleic acids encoding Cas protein and nucleic acids encoding the at least one to two ribonucleic acids are introduced into a cell via viral transduction (e.g., lentiviral transduction). In some embodiments, the Cas protein is complexed with 1-2 ribonucleic acids. In some embodiments, the Cas protein is complexed with two ribonucleic acids. In some embodiments, the Cas protein is complexed with one ribonucleic acid. In some embodiments, the Cas protein is encoded by a modified nucleic acid, as described herein (e.g., a synthetic, modified mRNA).

Exemplary gRNA sequences useful for CRISPR/Cas-based targeting of genes described herein are provided in Table 1. The sequences can be found in WO2016183041 filed May 9, 2016, the disclosure including the Tables, Appendices, and Sequence Listing is incorporated herein by reference in its entirety.

TABLE 1
Exemplary gRNA sequences useful for targeting genes

| Gene Name | SEQ ID NO: (WO2016183041) | WO2016183041 |
|---|---|---|
| HLA-A | SEQ ID NOs: 2-1418 | Table 8, Appendix 1 |
| HLA-B | SEQ ID NOs: 1419-3277 | Table 9, Appendix 2 |
| HLA-C | SEQ ID NOS: 3278-5183 | Table 10, Appendix 3 |
| RFX-ANK | SEQ ID NOs: 95636-102318 | Table 11, Appendix 4 |
| NFY-A | SEQ ID NOs: 102319-121796 | Table 13, Appendix 6 |
| RFX5 | SEQ ID NOs: 85645-90115 | Table 16, Appendix 9 |
| RFX-AP | SEQ ID NOs: 90116-95635 | Table 17, Appendix 10 |
| NFY-B | SEQ ID NOs: 121797-135112 | Table 20, Appendix 13 |
| NFY-C | SEQ ID NOs: 135113-176601 | Table 22, Appendix 15 |
| IRF1 | SEQ ID NOs: 176602-182813 | Table 23, Appendix 16 |
| TAP1 | SEQ ID NOs: 182814-188371 | Table 24, Appendix 17 |
| CIITA | SEQ ID NOS: 5184-36352 | Table 12, Appendix 5 |
| B2M | SEQ ID NOS: 81240-85644 | Table 15, Appendix 8 |
| NLRC5 | SEQ ID NOS: 36353-81239 | Table 14, Appendix 7 |
| CD47 | SEQ ID NOS: 200784-231885 | Table 29, Appendix 22 |
| HLA-E | SEQ ID NOS: 189859-193183 | Table 19, Appendix 12 |
| HLA-F | SEQ ID NOS: 688808-699754 | Table 45, Appendix 38 |
| HLA-G | SEQ ID NOS: 188372-189858 | Table 18, Appendix 11 |
| PD-L1 | SEQ ID NOS: 193184-200783 | Table 21, Appendix 14 |

In some embodiments, the cells of the invention are made using Transcription Activator-Like Effector Nucleases (TALEN) methodologies.

By a “TALE-nuclease” (TALEN) is intended a fusion protein consisting of a nucleic acid-binding domain typically derived from a Transcription Activator Like Effector (TALE) and one nuclease catalytic domain to cleave a nucleic acid target sequence. The catalytic domain is preferably a nuclease domain and more preferably a domain having endonuclease activity, like for instance I-TevI, ColE7, NucA and Fok-I. In a particular embodiment, the TALE domain can be fused to a meganuclease like for instance I-CreI and I-OnuI or functional variant thereof. In a more preferred embodiment, said nuclease is a monomeric TALE-Nuclease. A monomeric TALE-Nuclease is a TALE-Nuclease that does not require dimerization for specific recognition and cleavage, such as the fusions of engineered TAL repeats with the catalytic domain of I-TevI described in WO2012138927. Transcription Activator like Effector (TALE) are proteins from the bacterial species Xanthomonas comprise a plurality of repeated sequences, each repeat comprising di-residues in position 12 and 13 (RVD) that are specific to each nucleotide base of the nucleic acid targeted sequence. Binding domains with similar modular base-per-base nucleic acid binding properties (MBBBD) can also be derived from new modular proteins recently discovered by the applicant in a different bacterial species. The new modular proteins have the advantage of displaying more sequence variability than TAL repeats. Preferably, RVDs associated with recognition of the different nucleotides are HD for recognizing C, NG for recognizing T, NI for recognizing A, NN for recognizing G or A, NS for recognizing A, C, G or T, HG for recognizing T, IG for recognizing T, NK for recognizing G, HA for recognizing C, ND for recognizing C, HI for recognizing C, HN for recognizing G, NA for recognizing G, SN for recognizing G or A and YG for recognizing T, TL for recognizing A, VT for recognizing A or G and SW for recognizing A. In another embodiment, critical amino acids 12 and 13 can be mutated towards other amino acid residues in order to modulate their specificity towards nucleotides A, T, C and G and in particular to enhance this specificity. TALEN kits are sold commercially.

In some embodiments, the cells are manipulated using zinc finger nuclease (ZFN). A “zinc finger binding protein” is a protein or polypeptide that binds DNA, RNA and/or protein, preferably in a sequence-specific manner, as a result of stabilization of protein structure through coordination of a zinc ion. The term zinc finger binding protein is often abbreviated as zinc finger protein or ZFP. The individual DNA binding domains are typically referred to as “fingers.” A ZFP has least one finger, typically two fingers, three fingers, or six fingers. Each finger binds from two to four base pairs of DNA, typically three or four base pairs of DNA. A ZFP binds to a nucleic acid sequence called a target site or target segment. Each finger typically comprises an approximately 30 amino acid, zinc-chelating, DNA-binding subdomain. Studies have demonstrated that a single zinc finger of this class consists of an alpha helix containing the two invariant histidine residues coordinated with zinc along with the two cysteine residues of a single beta turn (see, e.g., Berg & Shi, Science 271:1081-1085 (1996)).

In some embodiments, the cells of the invention are made using a homing endonuclease. Such homing endonucleases are well-known to the art (Stoddard 2005). Homing endonucleases recognize a DNA target sequence and generate a single- or double-strand break. Homing endonucleases are highly specific, recognizing DNA target sites ranging from 12 to 45 base pairs (bp) in length, usually ranging from 14 to 40 bp in length. The homing endonuclease according to the invention may for example correspond to a LAGLIDADG endonuclease, to a HNH endonuclease, or to a GIY-YIG endonuclease. Preferred homing endonuclease according to the present invention can be an I-CreI variant.

In some embodiments, the cells of the invention are made using a meganuclease. Meganucleases are by definition sequence-specific endonucleases recognizing large sequences (Chevalier, B. S. and B. L. Stoddard, Nucleic Acids Res., 2001, 29, 3757-3774). They can cleave unique sites in living cells, thereby enhancing gene targeting by 1000-fold or more in the vicinity of the cleavage site (Puchta et al., Nucleic Acids Res., 1993, 21, 5034-5040; Rouet et al., Mol. Cell. Biol., 1994, 14, 8096-8106; Choulika et al., Mol. Cell. Biol., 1995, 15, 1968-1973; Puchta et al., Proc. Natl. Acad. Sci. USA, 1996, 93, 5055-5060; Sargent et al., Mol. Cell. Biol., 1997, 17, 267-77; Donoho et al., Mol. Cell. Biol, 1998, 18, 4070-4078; Elliott et al., Mol. Cell. Biol., 1998, 18, 93-101; Cohen-Tannoudji et al., Mol. Cell. Biol., 1998, 18, 1444-1448).

In some embodiments, the cells of the invention are made using RNA silencing or RNA interference (RNAi) to knockdown (e.g., decrease, eliminate, or inhibit) the expression of a polypeptide such as a tolerogenic factor. Useful RNAi methods include those that utilize synthetic RNAi molecules, short interfering RNAs (siRNAs), PIWI-interacting NRAs (piRNAs), short hairpin RNAs (shRNAs), microRNAs (miRNAs), and other transient knockdown methods recognized by those skilled in the art. Reagents for RNAi including sequence specific shRNAs, siRNA, miRNAs and the like are commercially available. For instance, CIITA can be knocked down in a pluripotent stem cell by introducing a CIITA siRNA or transducing a CIITA shRNA-expressing virus into the cell. In some embodiments, RNA interference is employed to reduce or inhibit the expression of at least one selected from the group consisting of CIITA, B2M, and NLRC5.

In some embodiments, cells of the present invention are genetically modified to reduce expression of one or more immune factors (including target polypeptides) to create immune-privileged or hypoimmunogenic cells. In certain embodiments, the cells (e.g., stem cells, induced pluripotent stem cells, differentiated cells, hematopoietic stem cells, primary T cells and CAR-T cells) disclosed herein comprise one or more genetic modifications to reduce expression of one or more target polynucleotides. Non-limiting examples of such target polynucleotides and polypeptides include CIITA, B2M, NLRC5, CTLA4, PD1, HLA-A, HLA-BM, HLA-C, RFX-ANK, NFY-A, RFX5, RFX-AP, NFY-B, NFY-C, IRF1, and TAP1.

In some aspects, the genetic modification occurs using a CRISPR/Cas system. By modulating (e.g., reducing or deleting) expression of one or a plurality of the target polynucleotides, such cells exhibit decreased immune activation when engrafted into a recipient subject. In some embodiments, the cell is considered hypoimmunogenic, e.g., in a recipient subject or patient upon administration.

### K. Methods of Overexpression of Tolerogenic Factors

Provided herein are cells that do not trigger or activate an immune response upon administration to a recipient subject. As described above, in some embodiments, the cells are modified to increase expression of genes and tolerogenic (e.g., immune) factors that affect immune recognition and tolerance in a recipient.

In certain embodiments, any of the cells (e.g., stem cells, induced pluripotent stem cells, differentiated cells, hematopoietic stem cells, primary T cells and CAR-T cells) disclosed herein that harbor a genomic modification that modulates expression of one or more target proteins listed herein are also modified to express one or more tolerogenic factors. Exemplary tolerogenic factors include, without limitation, one or more of CD24, CD47, DUX4, CD27, CD35, CD46, CD55, CD59, CD200, HLA-C, HLA-E, HLA-E heavy chain, HLA-G, PD-L1, IDO1, CTLA4-Ig, C1-Inhibitor, IL-10, IL-35, FasL, CCL21, CCL22, Mfge8, and Serpinb9. In some embodiments, the tolerogenic factors are selected from a group including CD24, CD47, CD27, CD35, CD46, CD55, CD59, CD200, HLA-C, HLA-E, HLA-E heavy chain, HLA-G, PD-L1, IDO1, CTLA4-Ig, C1-Inhibitor, IL-10, IL-35, FasL, CCL21, CCL22, Mfge8, and Serpinb9.

Useful genomic, polynucleotide and polypeptide information about human CD27 (which is also known as CD27L receptor, Tumor Necrosis Factor Receptor Superfamily Member 7, TNFSF7, T Cell Activation Antigen 5152, Tp55, and T14) are provided in, for example, the GeneCard Identifier GC12P008144, HGNC No. 11922, NCBI Gene ID 939, Uniprot No. P26842, and NCBI RefSeq Nos. NM_001242.4 and NP_001233.1.

Useful genomic, polynucleotide and polypeptide information about human CD46 are provided in, for example, the GeneCard Identifier GC01P207752, HGNC No. 6953, NCBI Gene ID 4179, Uniprot No. P15529, and NCBI RefSeq Nos. NM_002389.4, NM_153826.3, NM_172350.2, NM_172351.2, NM_172352.2 NP_758860.1, NM_172353.2, NM_172359.2, NM_172361.2, NP_002380.3, NP_722548.1, NP_758860.1, NP_758861.1, NP_758862.1, NP_758863.1, NP_758869.1, and NP_758871.1.

Useful genomic, polynucleotide and polypeptide information about human CD55 (also known as complement decay-accelerating factor) are provided in, for example, the GeneCard Identifier GC01P207321, HGNC No. 2665, NCBI Gene ID 1604, Uniprot No. P08174, and NCBI RefSeq Nos. NM_000574.4, NM_001114752.2, NM_001300903.1, NM_001300904.1, NP_000565.1, NP_001108224.1, NP_001287832.1, and NP_001287833.1.

Useful genomic, polynucleotide and polypeptide information about human CD59 are provided in, for example, the GeneCard Identifier GC11M033704, HGNC No. 1689, NCBI Gene ID 966, Uniprot No. P13987, and NCBI RefSeq Nos. NP_000602.1, NM_000611.5, NP_001120695.1, NM_001127223.1, NP_001120697.1, NM_001127225.1, NP_001120698.1, NM_001127226.1, NP_001120699.1, NM_001127227.1, NP_976074.1, NM_203329.2, NP_976075.1, NM_203330.2, NP_976076.1, and NM_203331.2.

Useful genomic, polynucleotide and polypeptide information about human CD200 are provided in, for example, the GeneCard Identifier GC03P112332, HGNC No. 7203, NCBI Gene ID 4345, Uniprot No. P41217, and NCBI RefSeq Nos. NP_001004196.2, NM_001004196.3, NP_001305757.1, NM_001318828.1, NP_005935.4, NM_005944.6, XP 005247539.1, and XM_005247482.2.

Useful genomic, polynucleotide and polypeptide information about human HLA-C are provided in, for example, the GeneCard Identifier GC06M031272, HGNC No. 4933, NCBI Gene ID 3107, UniprotNo. P10321, and NCBI RefSeq Nos. NP_002108.4 and NM_002117.5.

Useful genomic, polynucleotide and polypeptide information about human HLA-E are provided in, for example, the GeneCard Identifier GC06P047281, HGNC No. 4962, NCBI Gene ID 3133, UniprotNo. P13747, and NCBI RefSeq Nos. NP_005507.3 and NM_005516.5.

Useful genomic, polynucleotide and polypeptide information about human HLA-G are provided in, for example, the GeneCard Identifier GC06P047256, HGNC No. 4964, NCBI Gene ID 3135, UniprotNo. P17693, and NCBI RefSeq Nos. NP_002118.1 and NM_002127.5.

Useful genomic, polynucleotide and polypeptide information about human PD-L1 or CD274 are provided in, for example, the GeneCard Identifier GC09P005450, HGNC No. 17635, NCBI Gene ID 29126, Uniprot No. Q9NZQ7, and NCBI RefSeq Nos. NP_001254635.1, NM_001267706.1, NP_054862.1, and NM_014143.3.

Useful genomic, polynucleotide and polypeptide information about human IDO1 are provided in, for example, the GeneCard Identifier GC08P039891, HGNC No. 6059, NCBI Gene ID 3620, Uniprot No. P14902, and NCBI RefSeq Nos. NP_002155.1 and NM_002164.5.

Useful genomic, polynucleotide and polypeptide information about human IL-10 are provided in, for example, the GeneCard Identifier GC01M206767, HGNC No. 5962, NCBI Gene ID 3586, UniprotNo. P22301, and NCBI RefSeq Nos. NP_000563.1 and NM_000572.2.

Useful genomic, polynucleotide and polypeptide information about human Fas ligand (which is known as FasL, FASLG, CD178, TNFSF6, and the like) are provided in, for example, the GeneCard Identifier GC01P172628, HGNC No. 11936, NCBI Gene ID 356, UniprotNo. P48023, and NCBI RefSeq Nos. NP_000630.1, NM_000639.2, NP_001289675.1, and NM_001302746.1.

Useful genomic, polynucleotide and polypeptide information about human CCL21 are provided in, for example, the GeneCard Identifier GC09M034709, HGNC No. 10620, NCBI Gene ID 6366, UniprotNo. 000585, and NCBI RefSeq Nos. NP_002980.1 and NM_002989.3.

Useful genomic, polynucleotide and polypeptide information about human CCL22 are provided in, for example, the GeneCard Identifier GC16P057359, HGNC No. 10621, NCBI Gene ID 6367, UniprotNo. 000626, and NCBI RefSeq Nos. NP_002981.2, NM_002990.4, XP 016879020.1, and XM_017023531.1.

Useful genomic, polynucleotide and polypeptide information about human Mfge8 are provided in, for example, the GeneCard Identifier GC15M088898, HGNC No. 7036, NCBI Gene ID 4240, Uniprot No. Q08431, and NCBI RefSeq Nos. NP_001108086.1, NM_001114614.2, NP_001297248.1, NM_001310319.1, NP_001297249.1, NM_001310320.1, NP_001297250.1, NM_001310321.1, NP_005919.2, and NM_005928.3.

Useful genomic, polynucleotide and polypeptide information about human SerpinB9 are provided in, for example, the GeneCard Identifier GC06M002887, HGNC No. 8955, NCBI Gene ID 5272, Uniprot No. P50453, and NCBI RefSeq Nos. NP_004146.1, NM_004155.5, XP 005249241.1, and XM_005249184.4.

Useful genomic, polynucleotide and polypeptide information about human CD35 (also known as complement receptor type 1 (CR1), C3b/C4b receptor (C3BR), C4Br, knops blood group antigen, and C3-binding domain) are provided in, for example, the GeneCard Identifier GC01P207496, HGNC No. 2334, NCBI Gene ID 1378, Uniprot No. P17927, and NCBI RefSeq Nos. NP_000564.2, NM_000573.3, NP_000642.3, and NM_000651.4.

Methods for modulating expression of genes and factors (proteins) include genome editing technologies, and, RNA or protein expression technologies and the like. For all of these technologies, well known recombinant techniques are used, to generate recombinant nucleic acids as outlined herein.

In certain embodiments, the recombinant nucleic acids encoding a tolerogenic factor may be operably linked to one or more regulatory nucleotide sequences in an expression construct. Regulatory nucleotide sequences will generally be appropriate for the host cell and recipient subject to be treated. Numerous types of appropriate expression vectors and suitable regulatory sequences are known in the art for a variety of host cells. Typically, the one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are also contemplated. The promoters may be either naturally occurring promoters, or hybrid promoters that combine elements of more than one promoter. An expression construct may be present in a cell on an episome, such as a plasmid, or the expression construct may be inserted in a chromosome. In a specific embodiment, the expression vector includes a selectable marker gene to allow the selection of transformed host cells. Certain embodiments include an expression vector comprising a nucleotide sequence encoding a variant polypeptide operably linked to at least one regulatory sequence. Regulatory sequence for use herein include promoters, enhancers, and other expression control elements. In certain embodiments, an expression vector is designed for the choice of the host cell to be transformed, the particular variant polypeptide desired to be expressed, the vector's copy number, the ability to control that copy number, or the expression of any other protein encoded by the vector, such as antibiotic markers.

Examples of suitable mammalian promoters include, for example, promoters from the following genes: ubiquitin/S27a promoter of the hamster (WO 97/15664), Simian vacuolating virus 40 (SV40) early promoter, adenovirus major late promoter, mouse metallothionein-I promoter, the long terminal repeat region of Rous Sarcoma Virus (RSV), mouse mammary tumor virus promoter (MMTV), Moloney murine leukemia virus Long Terminal repeat region, and the early promoter of human Cytomegalovirus (CMV). Examples of other heterologous mammalian promoters are the actin, immunoglobulin or heat shock promoter(s). In additional embodiments, promoters for use in mammalian host cells can be obtained from the genomes of viruses such as polyoma virus, fowlpox virus (UK 2,211,504 published 5 Jul. 1989), bovine papilloma virus, avian sarcoma virus, cytomegalovirus, a retrovirus, hepatitis-B virus and Simian Virus 40 (SV40). In further embodiments, heterologous mammalian promoters are used. Examples include the actin promoter, an immunoglobulin promoter, and heat-shock promoters. The early and late promoters of SV40 are conveniently obtained as an SV40 restriction fragment which also contains the SV40 viral origin of replication (Fiers et al, Nature 273: 113-120 (1978)). The immediate early promoter of the human cytomegalovirus is conveniently obtained as a Hindlll E restriction fragment (Greenaway et al, Gene 18: 355-360 (1982)). The foregoing references are incorporated by reference in their entirety.

In some embodiments, expression of a target gene (e.g., CD24, CD47, or another tolerogenic factor) is increased by expression of fusion protein or a protein complex containing (1) a site-specific binding domain specific for the endogenous CD24, CD47, or other gene and (2) a transcriptional activator.

In some embodiments, the regulatory factor is comprised of a site specific DNA-binding nucleic acid molecule, such as a guide RNA (gRNA). In some embodiments, the method is achieved by site specific DNA-binding targeted proteins, such as zinc finger proteins (ZFP) or fusion proteins containing ZFP, which are also known as zinc finger nucleases (ZFNs).

In some aspects, the regulatory factor comprises a site-specific binding domain, such as using a DNA binding protein or DNA-binding nucleic acid, which specifically binds to or hybridizes to the gene at a targeted region. In some aspects, the provided polynucleotides or polypeptides are coupled to or complexed with a site-specific nuclease, such as a modified nuclease. For example, in some embodiments, the administration is effected using a fusion comprising a DNA-targeting protein of a modified nuclease, such as a meganuclease or an RNA-guided nuclease such as a clustered regularly interspersed short palindromic nucleic acid (CRISPR)-Cas system, such as CRISPR-Cas9 system. In some embodiments, the nuclease is modified to lack nuclease activity. In some embodiments, the modified nuclease is a catalytically dead dCas9.

In some embodiments, the site specific binding domain may be derived from a nuclease. For example, the recognition sequences of homing endonucleases and meganucleases such as I-SceI, I-CeuI, PI-PspI, PI-Sce, I-SceIV, I-CsmI, I-PanI, I-SceII, I-Ppol, I-SceIII, I-CreI, I-TevI, I-TevII and I-TevIII. See also U.S. Pat. Nos. 5,420,032; 6,833,252; Belfort et al., (1997) Nucleic Acids Res. 25:3379-3388; Dujon et al., (1989) Gene 82:115-118; Perler et al, (1994) Nucleic Acids Res. 22, 1125-1127; Jasin (1996) Trends Genet. 12:224-228; Gimble et al., (1996) J. Mol. Biol. 263:163-180; Argast et al, (1998) J. Mol. Biol. 280:345-353 and the New England Biolabs catalogue. In addition, the DNA-binding specificity of homing endonucleases and meganucleases can be engineered to bind non-natural target sites. See, for example, Chevalier et al, (2002) Molec. Cell 10:895-905; Epinat et al, (2003) Nucleic Acids Res. 31:2952-2962; Ashworth et al, (2006) Nature 441:656-659; Paques et al, (2007) Current Gene Therapy 7:49-66; U.S. Patent Publication No. 2007/0117128.

Zinc finger, TALE, and CRISPR system binding domains can be “engineered” to bind to a predetermined nucleotide sequence, for example via engineering (altering one or more amino acids) of the recognition helix region of a naturally occurring zinc finger or TALE protein. Engineered DNA binding proteins (zinc fingers or TALEs) are proteins that are non-naturally occurring. Rational criteria for design include application of substitution rules and computerized algorithms for processing information in a database storing information of existing ZFP and/or TALE designs and binding data. See, for example, U.S. Pat. Nos. 6,140,081; 6,453,242; and 6,534,261; see also WO 98/53058; WO 98/53059; WO 98/53060; WO 02/016536 and WO 03/016496 and U.S. Publication No. 20110301073.

In some embodiments, the site-specific binding domain comprises one or more zinc-finger proteins (ZFPs) or domains thereof that bind to DNA in a sequence-specific manner. A ZFP or domain thereof is a protein or domain within a larger protein that binds DNA in a sequence-specific manner through one or more zinc fingers, regions of amino acid sequence within the binding domain whose structure is stabilized through coordination of a zinc ion.

Among the ZFPs are artificial ZFP domains targeting specific DNA sequences, typically 9-18 nucleotides long, generated by assembly of individual fingers. ZFPs include those in which a single finger domain is approximately 30 amino acids in length and contains an alpha helix containing two invariant histidine residues coordinated through zinc with two cysteines of a single beta turn, and having two, three, four, five, or six fingers. Generally, sequence-specificity of a ZFP may be altered by making amino acid substitutions at the four helix positions (−1, 2, 3 and 6) on a zinc finger recognition helix. Thus, in some embodiments, the ZFP or ZFP-containing molecule is non-naturally occurring, e.g., is engineered to bind to a target site of choice. See, for example, Beerli et al. (2002) Nature Biotechnol. 20:135-141; Pabo et al. (2001) Ann. Rev. Biochem. 70:313-340; Isalan et al. (2001) Nature Biotechnol. 19:656-660; Segal et al. (2001) Curr. Opin. Biotechnol. 12:632-637; Choo et al. (2000) Curr. Opin. Struct. Biol. 10:411-416; U.S. Pat. Nos. 6,453,242; 6,534,261; 6,599,692; 6,503,717; 6,689,558; 7,030,215; 6,794,136; 7,067,317; 7,262,054; 7,070,934; 7,361,635; 7,253,273; and U.S. Patent Publication Nos. 2005/0064474; 2007/0218528; 2005/0267061, all incorporated herein by reference in their entireties.

Many gene-specific engineered zinc fingers are available commercially. For example, Sangamo Biosciences (Richmond, Calif., USA) has developed a platform (CompoZr) for zinc-finger construction in partnership with Sigma-Aldrich (St. Louis, Mo., USA), allowing investigators to bypass zinc-finger construction and validation altogether, and provides specifically targeted zinc fingers for thousands of proteins (Gaj et al., Trends in Biotechnology, 2013, 31(7), 397-405). In some embodiments, commercially available zinc fingers are used or are custom designed.

In some embodiments, the site-specific binding domain comprises a naturally occurring or engineered (non-naturally occurring) transcription activator-like protein (TAL) DNA binding domain, such as in a transcription activator-like protein effector (TALE) protein, See, e.g., U.S. Patent Publication No. 20110301073, incorporated by reference in its entirety herein.

In some embodiments, the site-specific binding domain is derived from the CRISPR/Cas system. In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system, or a “targeting sequence”), and/or other sequences and transcripts from a CRISPR locus.

In general, a guide sequence includes a targeting domain comprising a polynucleotide sequence having sufficient complementarity with a target polynucleotide sequence to hybridize with the target sequence and direct sequence-specific binding of the CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. In some examples, the targeting domain of the gRNA is complementary, e.g., at least 80, 85, 90, 95, 98 or 99% complementary, e.g., fully complementary, to the target sequence on the target nucleic acid.

In some embodiments, the target site is upstream of a transcription initiation site of the target gene. In some aspects, the target site is adjacent to a transcription initiation site of the gene. In some aspects, the target site is adjacent to an RNA polymerase pause site downstream of a transcription initiation site of the gene.

In some embodiments, the targeting domain is configured to target the promoter region of the target gene to promote transcription initiation, binding of one or more transcription enhancers or activators, and/or RNA polymerase. One or more gRNA can be used to target the promoter region of the gene. In some embodiments, one or more regions of the gene can be targeted. In certain aspects, the target sites are within 600 base pairs on either side of a transcription start site (TSS) of the gene.

It is within the level of a skilled artisan to design or identify a gRNA sequence that is or comprises a sequence targeting a gene, including the exon sequence and sequences of regulatory regions, including promoters and activators. A genome-wide gRNA database for CRISPR genome editing is publicly available, which contains exemplary single guide RNA (sgRNA) target sequences in constitutive exons of genes in the human genome or mouse genome (see e.g., genescript.com/gRNA-database.html; see also, Sanjana et al. (2014) Nat. Methods, 11:783-4; www.e-crisp.org/E-CRISP/; crispr.mit.edu/). In some embodiments, the gRNA sequence is or comprises a sequence with minimal off-target binding to a non-target gene.

In some embodiments, the regulatory factor further comprises a functional domain, e.g., a transcriptional activator.

A In some embodiments, the transcriptional activator is or contains one or more regulatory elements, such as one or more transcriptional control elements of a target gene, whereby a site-specific domain as provided above is recognized to drive expression of such gene. In some embodiments, the transcriptional activator drives expression of the target gene. In some cases, the transcriptional activator, can be or contain all or a portion of an heterologous transactivation domain. For example, in some embodiments, the transcriptional activator is selected from Herpes simplex-derived transactivation domain, Dnmt3a methyltransferase domain, p65, VP16, and VP64.

In some embodiments, the regulatory factor is a zinc finger transcription factor (ZF-TF). In some embodiments, the regulatory factor is VP64-p65-Rta (VPR).

In certain embodiments, the regulatory factor further comprises a transcriptional regulatory domain. Common domains include, e.g., transcription factor domains (activators, repressors, co-activators, co-repressors), silencers, oncogenes (e.g., myc, jun, fos, myb, max, mad, rel, ets, bcl, myb, mos family members etc.); DNA repair enzymes and their associated factors and modifiers; DNA rearrangement enzymes and their associated factors and modifiers; chromatin associated proteins and their modifiers (e.g. kinases, acetylases and deacetylases); and DNA modifying enzymes (e.g., methyltransferases such as members of the DNMT family (e.g., DNMT1, DNMT3A, DNMT3B, DNMT3L, etc., topoisomerases, helicases, ligases, kinases, phosphatases, polymerases, endonucleases) and their associated factors and modifiers. See, e.g., U.S. Publication No. 2013/0253040, incorporated by reference in its entirety herein.

Suitable domains for achieving activation include the HSV VP 16 activation domain (see, e.g., Hagmann et al, J. Virol. 71, 5952-5962 (1 97)) nuclear hormone receptors (see, e.g., Torchia et al., Curr. Opin. Cell. Biol. 10:373-383 (1998)); the p65 subunit of nuclear factor kappa B (Bitko & Bank, J. Virol. 72:5610-5618 (1998) and Doyle & Hunt, Neuroreport 8:2937-2942 (1997)); Liu et al., Cancer Gene Ther. 5:3-28 (1998)), or artificial chimeric functional domains such as VP64 (Beerli et al., (1998) Proc. Natl. Acad. Sci. USA 95:14623-33), and degron (Molinari et al., (1999) EMBO J. 18, 6439-6447). Additional exemplary activation domains include, Oct 1, Oct-2A, Sp1, AP-2, and CTF1 (Seipel etal, EMBOJ. 11, 4961-4968 (1992) as well as p300, CBP, PCAF, SRC1 PvALF, AtHD2A and ERF-2. See, for example, Robyr et al, (2000) Mol. Endocrinol. 14:329-347; Collingwood et al, (1999) J. Mol. Endocrinol 23:255-275; Leo et al, (2000) Gene 245:1-11; Manteuffel-Cymborowska (1999) Acta Biochim. Pol. 46:77-89; McKenna et al, (1999) J. Steroid Biochem. Mol. Biol. 69:3-12; Malik et al, (2000) Trends Biochem. Sci. 25:277-283; and Lemon et al, (1999) Curr. Opin. Genet. Dev. 9:499-504. Additional exemplary activation domains include, but are not limited to, OsGAI, HALF-1, Cl, AP1, ARF-5, -6, -1, and -8, CPRF1, CPRF4, MYC-RP/GP, and TRAB1, See, for example, Ogawa et al, (2000) Gene 245:21-29; Okanami et al, (1996) Genes Cells 1:87-99; Goff et al, (1991) Genes Dev. 5:298-309; Cho et al, (1999) Plant Mol Biol 40:419-429; Ulmason et al, (1999) Proc. Natl. Acad. Sci. USA 96:5844-5849; Sprenger-Haussels et al, (2000) Plant J. 22:1-8; Gong et al, (1999) Plant Mol. Biol. 41:33-44; and Hobo et al., (1999) Proc. Natl. Acad. Sci. USA 96:15,348-15,353.

Exemplary repression domains that can be used to make genetic repressors include, but are not limited to, KRAB A/B, KOX, TGF-beta-inducible early gene (TIEG), v-erbA, SID, MBD2, MBD3, members of the DNMT family (e.g., DNMT1, DNMT3A, DNMT3B, DNMT3L, etc.), Rb, and MeCP2. See, for example, Bird et al, (1999) Cell 99:451-454; Tyler et al, (1999) Cell 99:443-446; Knoepfler et al, (1999) Cell 99:447-450; and Robertson et al, (2000) Nature Genet. 25:338-342. Additional exemplary repression domains include, but are not limited to, ROM2 and AtHD2A. See, for example, Chem et al, (1996) Plant Cell 8:305-321; and Wu et al, (2000) Plant J. 22:19-27.

In some instances, the domain is involved in epigenetic regulation of a chromosome. In some embodiments, the domain is a histone acetyltransferase (HAT), e.g. type-A, nuclear localized such as MYST family members MOZ, Ybf2/Sas3, MOF, and Tip60, GNAT family members Gcn5 or pCAF, the p300 family members CBP, p300 or Rtt109 (Bemdsen and Denu (2008) Curr Opin Struct Biol 18(6):682-689). In other instances the domain is a histone deacetylase (HD AC) such as the class I (HDAC-1, 2, 3, and 8), class II (HDAC IIA (HDAC-4, 5, 7 and 9), HD AC IIB (HDAC 6 and 10)), class IV (HDAC-11), class III (also known as sirtuins (SIRTs); SIRT1-7) (see Mottamal et al., (2015) Molecules 20(3):3898-3941). Another domain that is used in some embodiments is a histone phosphorylase or kinase, where examples include MSK1, MSK2, ATR, ATM, DNA-PK, Bubl, VprBP, IKK-a, PKCpi, Dik/Zip, JAK2, PKCS, WSTF and CK2. In some embodiments, a methylation domain is used and may be chosen from groups such as Ezh2, PRMT1/6, PRMTS/7, PRMT 2/6, CARM1, set7/9, MLL, ALL-1, Suv 39h, G9a, SETDB1, Ezh2, Set2, Dot1, PRMT 1/6, PRMT 5/7, PR-Set7 and Suv4-20h, Domains involved in sumoylation and biotinylation (Lys9, 13, 4, 18 and 12) may also be used in some embodiments (review see Kousarides (2007) Cell 128:693-705).

Fusion molecules are constructed by methods of cloning and biochemical conjugation that are well known to those of skill in the art. Fusion molecules comprise a DNA-binding domain and a functional domain (e.g., a transcriptional activation or repression domain). Fusion molecules also optionally comprise nuclear localization signals (such as, for example, that from the SV40 medium T-antigen) and epitope tags (such as, for example, FLAG and hemagglutinin). Fusion proteins (and nucleic acids encoding them) are designed such that the translational reading frame is preserved among the components of the fusion.

Fusions between a polypeptide component of a functional domain (or a functional fragment thereof) on the one hand, and a non-protein DNA-binding domain (e.g., antibiotic, intercalator, minor groove binder, nucleic acid) on the other, are constructed by methods of biochemical conjugation known to those of skill in the art. See, for example, the Pierce Chemical Company (Rockford, Ill.) Catalogue. Methods and compositions for making fusions between a minor groove binder and a polypeptide have been described. Mapp et al, (2000) Proc. Natl. Acad. Sci. USA 97:3930-3935. Likewise, CRISPR/Cas TFs and nucleases comprising a sgRNA nucleic acid component in association with a polypeptide component function domain are also known to those of skill in the art and detailed herein.

The process of introducing the polynucleotides described herein into cells can be achieved by any suitable technique. Suitable techniques include calcium phosphate or lipid-mediated transfection, electroporation, and transduction or infection using a viral vector. In some embodiments, the polynucleotides are introduced into a cell via viral transduction (e.g., lentiviral transduction).

Once altered, the presence of expression of any of the molecule described herein can be assayed using known techniques, such as Western blots, ELISA assays, FACS assays, and the like.

In some embodiments, the invention provides hypoimmunogenic pluripotent cells that comprise a “suicide gene” or “suicide switch”. These are incorporated to function as a “safety switch” that can cause the death of the hypoimmunogenic pluripotent cells should they grow and divide in an undesired manner. The “suicide gene” ablation approach includes a suicide gene in a gene transfer vector encoding a protein that results in cell killing only when activated by a specific compound. A suicide gene may encode an enzyme that selectively converts a nontoxic compound into highly toxic metabolites. The result is specifically eliminating cells expressing the enzyme. In some embodiments, the suicide gene is the herpesvirus thymidine kinase (HSV-tk) gene and the trigger is ganciclovir. In other embodiments, the suicide gene is the Escherichia coli cytosine deaminase (EC-CD) gene and the trigger is 5-fluorocytosine (5-FC) (Barese et al., Mol. Therap. 20(10): 1932-1943 (2012), Xu et al, Cell Res. 8:73-8 (1998), both incorporated herein by reference in their entirety.)

In other embodiments, the suicide gene is an inducible Caspase protein. An inducible Caspase protein comprises at least a portion of a Caspase protein capable of inducing apoptosis. In preferred embodiments, the inducible Caspase protein is iCasp9. It comprises the sequence of the human FK506-binding protein, FKBP12, with an F36V mutation, connected through a series of amino acids to the gene encoding human caspase 9. FKBP12-F36V binds with high affinity to a small-molecule dimerizing agent, AP1903. Thus, the suicide function of iCasp9 in the instant invention is triggered by the administration of a chemical inducer of dimerization (CID). In some embodiments, the CID is the small molecule drug API 903. Dimerization causes the rapid induction of apoptosis. (See WO2011146862; Stasi et al, N. Engl. J. Med 365; 18 (2011); Tey et al, Biol. Blood Marrow Transplant. 13:913-924 (2007), each of which are incorporated by reference herein in their entirety.)

### L. Generation of Induced Pluripotent Stem Cells

The invention provides methods of producing hypoimmunogenic pluripotent cells. In some embodiments, the method comprises generating pluripotent stem cells. The generation of mouse and human pluripotent stem cells (generally referred to as iPSCs; miPSCs for murine cells or hiPSCs for human cells) is generally known in the art. As will be appreciated by those in the art, there are a variety of different methods for the generation of iPCSs. The original induction was done from mouse embryonic or adult fibroblasts using the viral introduction of four transcription factors, Oct3/4, Sox2, c-Myc and Klf4; see Takahashi and Yamanaka Cell 126:663-676 (2006), hereby incorporated by reference in its entirety and specifically for the techniques outlined therein. Since then, a number of methods have been developed; see Seki et al, World J. Stem Cells 7(1): 116-125 (2015) for a review, and Lakshmipathy and Vermuri, editors, Methods in Molecular Biology: Pluripotent Stem Cells, Methods and Protocols, Springer 2013, both of which are hereby expressly incorporated by reference in their entirety, and in particular for the methods for generating hiPSCs (see for example Chapter 3 of the latter reference).

Generally, iPSCs are generated by the transient expression of one or more reprogramming factors” in the host cell, usually introduced using episomal vectors. Under these conditions, small amounts of the cells are induced to become iPSCs (in general, the efficiency of this step is low, as no selection markers are used). Once the cells are “reprogrammed”, and become pluripotent, they lose the episomal vector(s) and produce the factors using the endogeneous genes.

As is also appreciated by those of skill in the art, the number of reprogramming factors that can be used or are used can vary. Commonly, when fewer reprogramming factors are used, the efficiency of the transformation of the cells to a pluripotent state goes down, as well as the “pluripotency”, e.g., fewer reprogramming factors may result in cells that are not fully pluripotent but may only be able to differentiate into fewer cell types.

In some embodiments, a single reprogramming factor, OCT4, is used. In other embodiments, two reprogramming factors, OCT4 and KLF4, are used. In other embodiments, three reprogramming factors, OCT4, KLF4 and SOX2, are used. In other embodiments, four reprogramming factors, OCT4, KLF4, SOX2 and c-Myc, are used. In other embodiments, 5, 6 or 7 reprogramming factors can be used selected from SOKMNLT; SOX2, OCT4 (POU5F1), KLF4, MYC, NANOG, LIN28, and SV40L T antigen. In general, these reprogramming factor genes are provided on episomal vectors such as are known in the art and commercially available.

In general, as is known in the art, iPSCs are made from non-pluripotent cells such as, but not limited to, blood cells, fibroblasts, etc., by transiently expressing the reprogramming factors as described herein.

### M. Assays for Hypoimmunogenicity Phenotypes and Retention of Pluripotency

Once the hypoimmunogenic cells have been generated, they may be assayed for their hypoimmunogenicity and/or retention of pluripotency as is described in WO2016183041 and WO2018132783.

In some embodiments, hypoimmunogenicity is assayed using a number of techniques as exemplified in FIG. 13 and FIG. 15 of WO2018132783. These techniques include transplantation into allogeneic hosts and monitoring for hypoimmunogenic pluripotent cell growth (e.g. teratomas) that escape the host immune system. In some instances, hypoimmunogenic pluripotent cell derivatives are transduced to express luciferase and can then followed using bioluminescence imaging. Similarly, the T cell and/or B cell response of the host animal to such cells are tested to confirm that the cells do not cause an immune reaction in the host animal. T cell function can be assessed by Elispot, ELISA, FACS, PCR, or mass cytometry (CYTOF). B cell response or antibody response is assessed using FACS or Luminex. Additionally or alternatively, the cells may be assayed for their ability to avoid innate immune responses, e.g., NK cell killing, as is generally shown in FIGS. 14 and 15 of WO2018132783.

In some embodiments, the immunogenicity of the cells is evaluated using T cell immunoassays such as T cell proliferation assays, T cell activation assays, and T cell killing assays recognized by those skilled in the art. In some cases, the T cell proliferation assay includes pretreating the cells with interferon-gamma and coculturing the cells with labelled T cells and assaying the presence of the T cell population (or the proliferating T cell population) after a preselected amount of time. In some cases, the T cell activation assay includes coculturing T cells with the cells outlined herein and determining the expression levels of T cell activation markers in the T cells.

In vivo assays can be performed to assess the immunogenicity of the cells outlined herein. In some embodiments, the survival and immunogenicity of hypoimmunogenic cells is determined using an allogenic humanized immunodeficient mouse model. In some instances, the hypoimmunogenic pluripotent stem cells are transplanted into an allogenic humanized NSG-SGM3 mouse and assayed for cell rejection, cell survival, and teratoma formation. In some instances, grafted hypoimmunogenic pluripotent stem cells or differentiated cells thereof display long-term survival in the mouse model.

Additional techniques for determining immunogenicity including hypoimmunogenicity of the cells are described in, for example, Deuse et al., Nature Biotechnology, 2019, 37, 252-258 and Han et al., Proc Natl Acad Sci USA, 2019, 116(21), 10441-10446, the disclosures including the figures, figure legends, and description of methods are incorporated herein by reference in their entirety.

Similarly, the retention of pluripotency is tested in a number of ways. In one embodiment, pluripotency is assayed by the expression of certain pluripotency-specific factors as generally described herein and shown in FIG. 29 of WO2018132783. Additionally or alternatively, the pluripotent cells are differentiated into one or more cell types as an indication of pluripotency.

As will be appreciated by those in the art, the successful reduction of the MHC I function (HLA I when the cells are derived from human cells) in the pluripotent cells can be measured using techniques known in the art and as described below; for example, FACS techniques using labeled antibodies that bind the HLA complex; for example, using commercially available HLA-A, B, C antibodies that bind to the alpha chain of the human major histocompatibility HLA Class I antigens.

In addition, the cells can be tested to confirm that the HLA I complex is not expressed on the cell surface. This may be assayed by FACS analysis using antibodies to one or more HLA cell surface components as discussed above.

The successful reduction of the MHC II function (HLA II when the cells are derived from human cells) in the pluripotent cells or their derivatives can be measured using techniques known in the art such as Western blotting using antibodies to the protein, FACS techniques, RT-PCR techniques, etc.

In addition, the cells can be tested to confirm that the HLA II complex is not expressed on the cell surface. Again, this assay is done as is known in the art (See FIG. 21 of WO2018132783, for example) and generally is done using either Western Blots or FACS analysis based on commercial antibodies that bind to human HLA Class II HLA-DR, DP and most DQ antigens.

In addition to the reduction of HLA I and II (or MHC I and II), the hypoimmunogenic cells of the invention have a reduced susceptibility to macrophage phagocytosis and NK cell killing. The resulting hypoimmunogenic cells “escape” the immune macrophage and innate pathways due to the expression of one or more CD24 transgenes.

### N. Maintenance of Hypoimmunogenic Pluripotent Stem Cells

Once the hypoimmunogenic pluripotent stem cells have been generated, they can be maintained an undifferentiated state as is known for maintaining iPSCs. For example, the cells can be cultured on Matrigel using culture media that prevents differentiation and maintains pluripotency. In addition, they can be in culture medium under conditions to maintain pluripotency.

### O. Differentiation of Pluripotent Stem Cells

The invention provides pluripotent stem cells that may be differentiated into different cell types for subsequent transplantation into recipient subjects. Differentiation can be assayed as is known in the art, generally by evaluating the presence of cell-specific markers. As will be appreciated by those in the art, the differentiated hypoimmunogenic pluripotent cell derivatives can be transplanted using techniques known in the art that depends on both the cell type and the ultimate use of these cells.

1. Cardiac Cells Differentiated from Pluripotent Stem Cells

The invention provides pluripotent stem cells that may be differentiated into different cardiac cell types for subsequent transplantation or engraftment into subjects (e.g., recipients). As will be appreciated by those in the art, the methods for differentiation depend on the desired cell type using known techniques. Exemplary cardiac cell types include, but are not limited to, a cardiomyocyte, nodal cardiomyocyte, conducting cardiomyocyte, working cardiomyocyte, cardiomyocyte precursor cell, cardiac stem cell, atrial cardiac stem cell, ventricular cardiac stem cell, epicardial cell, hematopoietic cell, vascular endothelial cell, endocardial endothelial cell, cardiac valve interstitial cell, cardiac pacemarker cell, and the like.

In some embodiments, the cardiomyocyte precursor includes a cell that is capable (without dedifferentiation or reprogramming) of giving rise to progeny that include mature (end-stage) cardiomyocytes. Cardiomyocyte precursor cells can often be identified using one or more markers selected from GATA-4, Nkx2.5, and the MEF-2 family of transcription factors. In some instances, cardiomyocytes refer to immature cardiomyocytes or mature cardiomyocytes that express one or more markers (sometimes at least 3 or 5 markers) from the following list: cardiac troponin I (cTnl), cardiac troponin T (cTnT), sarcomeric myosin heavy chain (MHC), GATA-4, Nkx2.5, N-cadherin, (32- adrenoceptor, ANF, the MEF-2 family of transcription factors, creatine kinase MB (CK-MB), myoglobin, or atrial natriuretic factor (ANF). In some embodiments, the cardiac cells demonstrate spontaneous periodic contractile activity. In some cases, when that cardiac cells are cultured in a suitable tissue culture environment with an appropriate Ca²⁺ concentration and electrolyte balance, the cells can be observed to contract in a periodic fashion across one axis of the cell, and then release from contraction, without having to add any additional components to the culture medium. In some embodiments, the cardiac cells are hypoimmunogenic cardiac cells.

In some embodiments, cardiac cells described herein are administered to a recipient subject to treat a cardiac disorder selected from the group consisting of pediatric cardiomyopathy, age-related cardiomyopathy, dilated cardiomyopathy, hypertrophic cardiomyopathy, restrictive cardiomyopathy, chronic ischemic cardiomyopathy, peripartum cardiomyopathy, inflammatory cardiomyopathy, idiopathic cardiomyopathy, other cardiomyopathy, myocardial ischemic reperfusion injury, ventricular dysfunction, heart failure, congestive heart failure, coronary artery disease, end stage heart disease, atherosclerosis, ischemia, hypertension, restenosis, angina pectoris, rheumatic heart, arterial inflammation, cardiovascular disease, myocardial infarction, myocardial ischemia, congestive heart failure, myocardial infarction, cardiac ischemia, cardiac injury, myocardial ischemia, vascular disease, acquired heart disease, congenital heart disease, atherosclerosis, coronary artery disease, dysfunctional conduction systems, dysfunctional coronary arteries, pulmonary hypertension, cardiac arrhythmias, muscular dystrophy, muscle mass abnormality, muscle degeneration, myocarditis, infective myocarditis, drug- or toxin-induced muscle abnormalities, hypersensitivity myocarditis, and autoimmune endocarditis.

In some embodiments, the method of producing a population of hypoimmunogenic cardiac cells from a population of hypoimmunogenic pluripotent (HIP) cells by in vitro differentiation comprises: (a) culturing a population of HIP cells in a culture medium comprising a GSK inhibitor; (b) culturing the population of HIP cells in a culture medium comprising a WNT antagonist to produce a population of pre-cardiac cells; and (c) culturing the population of pre-cardiac cells in a culture medium comprising insulin to produce a population of hypoimmune cardiac cells. In some embodiments, the GSK inhibitor is CHIR-99021, a derivative thereof, or a variant thereof. In some instances, the GSK inhibitor is at a concentration ranging from about 2 mM to about 10 mM. In some embodiments, the WNT antagonist is IWR1, a derivative thereof, or a variant thereof. In some instances, the WNT antagonist is at a concentration ranging from about 2 mM to about 10 mM.

In some embodiments, the population of hypoimmunogenic cardiac cells is isolated from non-cardiac cells. In some embodiments, the isolated population of hypoimmunogenic cardiac cells are expanded prior to administration. In certain embodiments, the isolated population of hypoimmunogenic cardiac cells are expanded and cryopreserved prior to administration.

Other useful methods for differentiating induced pluripotent stem cells or pluripotent stem cells into cardiac cells are described, for example, in US2017/0152485; US2017/0058263; US2017/0002325; US2016/0362661; US2016/0068814; U.S. Pat. Nos. 9,062,289; 7,897,389; and 7,452,718. Additional methods for producing cardiac cells from induced pluripotent stem cells or pluripotent stem cells are described in, for example, Xu et al, Stem Cells and Development, 2006, 15(5): 631-9, Burridge et al, Cell Stem Cell, 2012, 10: 16-28, and Chen et al, Stem Cell Res, 2015, 15(2):365-375.

In various embodiments, hypoimmunogenic cardiac cells can be cultured in culture medium comprising a BMP pathway inhibitor, a WNT signaling activator, a WNT signaling inhibitor, a WNT agonist, a WNT antagonist, a Src inhibitor, a EGFR inhibitor, a PCK activator, a cytokine, a growth factor, a cardiotropic agent, a compound, and the like.

The WNT signaling activator includes, but is not limited to, CHIR99021. The PCK activator includes, but is not limited to, PMA. The WNT signaling inhibitor includes, but is not limited to, a compound selected from KY02111, 503031 (KY01-I), SO2031 (KY02-I), and S03042 (KY03-D, and XAV939. The Src inhibitor includes, but is not limited to, A419259. The EGFR inhibitor includes, but is not limited to, AG1478.

Non-limiting examples of an agent for generating a cardiac cell from an iPSC include activin A, BMP-4, Wnt3a, VEGF, soluble frizzled protein, cyclosporin A, angiotensin II, phenylephrine, ascorbic acid, dimethylsulfoxide, 5-aza-2′-deoxycytidine, and the like.

The cells of the present invention can be cultured on a surface, such as a synthetic surface to support and/or promote differentiation of hypoimmunogenic pluripotent cells into cardiac cells. In some embodiments, the surface comprises a polymer material including, but not limited to, a homopolymer or copolymer of selected one or more acrylate monomers. Non-limiting examples of acrylate monomers and methacrylate monomers include tetra(ethylene glycol) diacrylate, glycerol dimethacrylate, 1,4-butanediol dimethacrylate, poly(ethylene glycol) diacrylate, di(ethylene glycol) dimethacrylate, tetra(ethyiene glycol) dimethacrylate, 1,6-hexanediol propoxylate diacrylate, neopentyl glycol diacrylate, trimethylolpropane benzoate diacrylate, trimethylolpropane eihoxylate (1 EO/QH) methyl, tricyclo[5.2.1.0^{2,6}] decane dimethanol diacrylate, neopentyl glycol exhoxylate diacrylate, and trimethylolpropane triacrylate. Acrylate synthesized as known in the art or obtained from a commercial vendor, such as Polysciences, Inc., Sigma Aldrich, Inc. and Sartomer, Inc.

The polymeric material can be dispersed on the surface of a support material. Useful support materials suitable for culturing cells include a ceramic substance, a glass, a plastic, a polymer or co-polymer, any combinations thereof, or a coating of one material on another. In some instances, a glass includes soda-lime glass, pyrex glass, vycor glass, quartz glass, silicon, or derivatives of these or the like.

In some instances, plastics or polymers including dendritic polymers include poly(vinyl chloride), poly(vinyl alcohol), poly(methyl methacrylate), poly(vinyl acetate-maleic anhydride), poly(dimethylsiloxane) monomethacrylate, cyclic olefin polymers, fluorocarbon polymers, polystyrenes, polypropylene, polyethyleneimine or derivatives of these or the like. In some instances, copolymers include poly(vinyl acetate-co-maleic anhydride), poly(styrene-co-maleic anhydride), poly(ethylene-co-acrylic acid) or derivatives of these or the like.

The efficacy of cardiac cells prepared as described herein can be assessed in animal models for cardiac cryoinjury, which causes 55% of the left ventricular wall tissue to become scar tissue without treatment (Li et al, Ann. Thorac. Surg. 62:654, 1996; Sakai et al, Ann. Thorac. Surg. 8:2074, 1999, Sakai et al., Thorac. Cardiovasc. Surg. 118:715, 1999). Successful treatment can reduce the area of the scar, limit scar expansion, and improve heart function as determined by systolic, diastolic, and developed pressure. Cardiac injury can also be modeled using an embolization coil in the distal portion of the left anterior descending artery (Watanabe et al., Cell Transplant. 7:239, 1998), and efficacy of treatment can be evaluated by histology and cardiac function.

In some embodiments, the administration comprises implantation into the subject's heart tissue, intravenous injection, intraarterial injection, intracoronary injection, intramuscular injection, intraperitoneal injection, intramyocardial injection, trans-endocardial injection, trans-epicardial injection, or infusion.

In some embodiments, the patient administered the engineered cardiac cells is also administered a cardiac drug. Illustrative examples of cardiac drugs that are suitable for use in combination therapy include, but are not limited to, growth factors, polynucleotides encoding growth factors, angiogenic agents, calcium channel blockers, antihypertensive agents, antimitotic agents, inotropic agents, anti-atherogenic agents, anti-coagulants, beta-blockers, anti-arhythmic agents, anti-inflammatory agents, vasodilators, thrombolytic agents, cardiac glycosides, antibiotics, antiviral agents, antifungal agents, agents that inhibit protozoans, nitrates, angiotensin converting enzyme (ACE) inhibitors, angiotensin II receptor antagonist, brain natriuretic peptide (BNP); antineoplastic agents, steroids, and the like.

The effects of therapy according to the methods of the invention can be monitored in a variety of ways. For instance, an electrocardiogram (ECG) or holier monitor can be utilized to determine the efficacy of treatment. An ECG is a measure of the heart rhythms and electrical impulses, and is a very effective and non-invasive way to determine if therapy has improved or maintained, prevented, or slowed degradation of the electrical conduction in a subject's heart. The use of a holier monitor, a portable ECG that can be worn for long periods of time to monitor heart abnormalities, arrhythmia disorders, and the like, is also a reliable method to assess the effectiveness of therapy. An ECG or nuclear study can be used to determine improvement in ventricular function.

2. Neural Cells Differentiated from Pluripotent Stem Cells

The invention provides pluripotent stem cells that may be differentiated into different neural cell types for subsequent transplantation or engraftment into recipient subjects. As will be appreciated by those in the art, the methods for differentiation depend on the desired cell type using known techniques. Exemplary neural cell types include, but are not limited to, cerebral endothelial cells, neurons, glial cells, and the like.

In some embodiments, neural cells are administered to a subject to treat Parkinson's disease, Huntington disease, multiple sclerosis, other neurodegenerative disease or condition, attention deficit hyperactivity disorder (ADHD), Tourette Syndrome (TS), schizophrenia, psychosis, depression, other neuropsychiatric disorder. In some embodiments, neural cells described herein are administered to a subject to treat or ameliorate stroke. In some embodiments, the neurons and glial cells are administered to a subject with amyotrophic lateral sclerosis (ALS). In some embodiments, cerebral endothelial cells are administered to alleviate the symptoms or effects of cerebral hemorrhage. In some embodiments, dopaminergic neurons are administered to a patient with Parkinson's disease. In some embodiments, noradrenergic neurons, GABAergic interneurons are administered to a patient who has experienced an epileptic seizure. In some embodiments, motor neurons, interneurons, Schwann cells, oligodendrocytes, and microglia are administered to a patient who has experienced a spinal cord injury.

In some embodiments, cerebral endothelial cells (ECs), precursors, and progenitors thereof are differentiated from pluripotent stem cells (e.g., induced pluripotent stem cells) on a surface by culturing the cells in a medium comprising one or more factors that promote the generation of cerebral ECs or neural cell. In some instances, the medium includes one or more of the following: CHIR-99021, VEGF, basic FGF, and Y-27632. In some embodiments, the medium includes a supplement designed to promote survival and functionality for neural cells.

In some embodiments, cerebral endothelial cells (ECs), precursors, and progenitors thereof are differentiated from pluripotent stem cells on a surface by culturing the cells in an unconditioned or conditioned medium. In some instances, the medium comprises factors or small molecules that promote or facilitate differentiation. In some embodiments, the medium comprises one or more factors or small molecules selected from the group consisting of VEGR, FGF, SDF-1, CHIR-99021, Y-27632, SB 431542, and any combination thereof. In some embodiments, the surface for differentiation comprises one or more extracellular matrix proteins. The surface can be coated with the one or more extracellular matrix proteins. The cells can be differentiated in suspension and then put into a gel matrix form, such as matrigel, gelatin, or fibrin/thrombin forms to facilitate cell survival. In some cases, differentiation is assayed as is known in the art, generally by evaluating the presence of cell-specific markers.

In some embodiments, the cerebral endothelial cells express or secrete a factor selected from the group consisting of CD31, VE cadherin, and a combination thereof. In certain embodiments, the cerebral endothelial cells express or secrete one or more of the factors selected from the group consisting of CD31, CD34, CD45, CD117 (c-kit), CD146, CXCR4, VEGF, SDF-1, PDGF, GLUT-1, PECAM-1, eNOS, claudin-5, occludin, ZO-1, p-glycoprotein, von Willebrand factor, VE-cadherin, low density lipoprotein receptor LDLR, low density lipoprotein receptor-related protein 1 LRP1, insulin receptor INSR, leptin receptor LEPR, basal cell adhesion molecule BCAM, transferrin receptor TFRC, advanced glycation endproduct-specific receptor AGER, receptor for retinol uptake STRA6, large neutral amino acids transporter small subunit 1 SLC7A5, excitatory amino acid transporter 3 SLC1A1, sodium-coupled neutral amino acid transporter 5 SLC38A5, solute carrier family 16 member 1 SLC16A1, ATP-dependent translocase ABCB1, ATP-ABCC2 binding cassette transporter ABCG2, multidrug resistance-associated protein 1 ABCC1, canalicular multispecific organic anion transporter 1 ABCC2, multidrug resistance-associated protein 4 ABCC4, and multidrug resistance-associated protein 5 ABCC5.

In some embodiments, the cerebral ECs are characterized with one or more of the features selected from the group consisting of high expression of tight junctions, high electrical resistance, low fenestration, small perivascular space, high prevalence of insulin and transferrin receptors, and high number of mitochondria.

In some embodiments, cerebral ECs are selected or purified using a positive selection strategy. In some instances, the cerebral ECs are sorted against an endothelial cell marker such as, but not limited to, CD31. In other words, CD31 positive cerebral ECs are isolated. In some embodiments, cerebral ECs are selected or purified using a negative selection strategy. In some embodiments, undifferentiated or pluripotent stem cells are removed by selecting for cells that express a pluripotency marker including, but not limited to, TRA-1-60 and SSEA-1.

In some embodiments, neurons, precursors, and progenitors thereof are differentiated from pluripotent stem cells by culturing the cells in medium comprising one or more factors selected from the group consisting of GDNF, BDNF, GM-CSF, B27, basic FGF, basic EGF, NGF, CNTF, SMAD inhibitor, Wnt antagonist, SHH signaling activator, and any combination thereof. In some embodiments, the SMAD inhibitor is selected from the group consisting of SB431542, LDN-193189, Noggin PD169316, SB203580, LY364947, A77-01, A-83-01, BMP4, GW788388, GW6604, SB-505124, lerdelimumab, metelimumab, GC-I008, AP-12009, AP-11014, LY550410, LY580276, LY364947, LY2109761, SB-505124, E-616452 (RepSox ALK inhibitor), SD-208, SMI6, NPC-30345, K 26894, SB-203580, SD-093, activin-M108A, P144, soluble TBR2-Fc, DMH-1, dorsomorphin dihydrochloride and derivatives thereof. In some embodiments, the Wnt antagonist is selected from the group consisting of XAV939, DKK1, DKK-2, DKK-3, Dkk-4, SFRP-1, SFRP-2, SFRP-5, SFRP-3, SFRP-4, WIF-1, Soggy, IWP-2, IWR1, ICG-001, KY0211, Wnt-059, LGK974, IWP-L6 and derivatives thereof. In some embodiments, the SHH signaling activator is selected from the group consisting of Smoothened agonist (SAG), SAG analog, SHH, C25-SHH, C24-SHH, purmorphamine, Hg—Ag and derivatives thereof.

In some embodiments, the neurons expression one or more of the markers selected from the group consisting of glutamate ionotropic receptor NMDA type subunit 1 GRIN1, glutamate decarboxylase 1 GAD1, gamma-aminobutyric acid GABA, tyrosine hydroxylase TH, LIM homeobox transcription factor 1-alpha LMX1A, Forkhead box protein 01 FOXO1, Forkhead box protein A2 FOXA2, Forkhead box protein 04 FOXO4, FOXG1, 2′,3′-cyclic-nucleotide 3′-phosphodiesterase CNP, myelin basic protein MBP, tubulin beta chain 3 TUB3, tubulin beta chain 3 NEUN, solute carrier family 1 member 6 SLC1A6, SST, PV, calbindin, RAX, LHX6, LHX8, DLX1, DLX2, DLX5, DLX6, SOX6, MAFB, NPAS1, ASCL1, SIX6, OLIG2, NKX2.1, NKX2.2, NKX6.2, VGLUT1, MAP2, CTIP2, SATB2, TBR1, DLX2, ASCL1, ChAT, NGFI-B, c-fos, CRF, RAX, POMC, hypocretin, NADPH, NGF, Ach, VAChT, PAX6, EMX2p75, CORIN, TUJ1, NURR1, and any combination thereof. In some embodiments, the dopaminergic neurons express one or more of the markers selected from CORIN, FOXA2, TUJ1, NURR1, and any combination thereof.

In some embodiments, stem cells described herein are differentiated into dopaminergic neurons include dopaminergic progenitors. The stem cells are cultured in a differentiation medium comprising a supplement or additive to induce neuronal differentiation. In some embodiments, the cells are cultured in the presence of a supplement or additive to induce floor plate cells. In some embodiments, the supplement or additive includes BMP inhibitor LDN193189, ALK-5 inhibitor A83-01, Smoothened agonist purmorphamine, FGF8, GSK3 inhibitor CHIR99021, glial cell line-derived neurotrophic factor, GDNF, ascorbic acid, brain-derived neurotrophic factor BDNF, dibutyryladenosine cyclic monophosphate dbcAMP, ROCK inhibitor Y-27632, and the like.

In some embodiments, the method of producing a population of hypoimmunogenic dopaminergic neurons from a population of hypoimmunogenic induced pluripotent stem cells (HIP cells) by in vitro differentiation comprises (a) culturing the population of HIP cells in a first culture medium comprising one or more factors selected from the group consisting of sonic hedgehog (SHH), BDNF, EGF, bFGF, FGF8, WNT1, retinoic acid, a GSK3 inhibitor, an ALK inhibitor, and a ROCK inhibitor to produce a population of immature dopaminergic neurons; and (b) culturing the population of immature dopaminergic neurons in a second culture medium that is different than the first culture medium to produce a population of dopaminergic neurons. In some embodiments, the GSK inhibitor is CHIR-99021, a derivative thereof, or a variant thereof. In some instances, the GSK inhibitor is at a concentration ranging from about 2 mM to about 10 pM. In some embodiments, the ALK inhibitor is SB-431542, a derivative thereof, or a variant thereof. In some instances, the ALK inhibitor is at a concentration ranging from about 1 mM to about 10 mM. In some embodiments, the first culture medium and/or second culture medium are absent of animal serum.

In some embodiments, the population of hypoimmunogenic dopaminergic neurons is isolated from non-neuronal cells. In some embodiments, the isolated population of hypoimmunogenic dopaminergic neurons are expanded prior to administration. In certain embodiments, the isolated population of hypoimmunogenic dopaminergic neurons are expanded and cryopreserved prior to administration.

Methods for differentiating pluripotent stem cells are described in, e.g., Kikuchi et al., Nature, 2017, 548, 592-596; Kriks et al., Nature, 2011, 547-551; Doi et al., Stem Cell Reports, 2014, 2, 337-50; Perrier et al., Proc Natl Acad Sci USA, 2004, 101, 12543-12548; Chambers et al., Nat Biotechnol, 2009, 27, 275-280; and Kirkeby et al., Cell Reports, 2012, 1, 703-714.

Useful descriptions of neurons derived from stem cells and methods of making thereof can be found, for example, in Kirkeby et al., Cell Rep, 2012, 1:703-714; Kriks et al., Nature, 2011, 480:547-551; Wang et al., Stem Cell Reports, 2018, 11(1):171-182; Lorenz Studer, “Chapter 8—Strategies for Bringing Stem Cell-Derived Dopamine Neurons to the clinic—The NYSTEM Trial” in Progress in Brain Research, 2017, volume 230, pg. 191-212; Liu et al., Nat Protoc, 2013, 8:1670-1679; Upadhya et al., Curr Protoc Stem Cell Biol, 38, 2D.7.1-2D.7.47; US Publication Appl. No. 20160115448, and U.S. Pat. Nos. 8,252,586; 8,273,570; 9,487,752 and 10,093,897, the contents are incorporated herein by reference in their entirety.

In some embodiments, glial cells including microglia, astrocytes, oligodendrocytes, ependymal cells and Schwann cells, glial precursors, and glial progenitors thereof are produced by differentiating pluripotent stem cells into therapeutically effective glial cells and the like. Differentiation of hypoimmunogenic pluripotent stem cells produces hypoimmunogenic neural cells, such as hypoimmunogenic glial cells.

In some embodiments, glial cells, precursors, and progenitors thereof generated by culturing pluripotent stem cells in medium comprising one or more agents selected from the group consisting of retinoic acid, IL-34, M-CSF, FLT3 ligand, GM-CSF, CCL2, a TGFbeta inhibitor, a BMP signaling inhibitor, a SHH signaling activator, FGF, platelet derived growth factor PDGF, PDGFR-alpha, HGF, IGF-1, noggin, sonic hedgehog (SHH), dorsomorphin, noggin, and any combination thereof. In certain instances, the BMP signaling inhibitor is LDN193189, SB431542, or a combination thereof. In some embodiments, the glial cells express NKX2.2, PAX6, SOX10, brain derived neurotrophic factor BDNF, neutrotrophin-3 NT-3, NT-4, epidermal growth factor EGF, ciliary neurotrophic factor CNTF, nerve growth factor NGF, FGF8, EGFR, OLIG1, OLIG2, myelin basic protein MBP, GAP-43, LNGFR, nestin, GFAP, CD11b, CD11c, CX3CR1, P2RY12, IBA-1, TMEM119, CD45, and any combination thereof. Exemplary differentiation medium can include any specific factors and/or small molecules that may facilitate or enable the generation of a glial cell type as recognized by those skilled in the art.

To determine if the cells generated according to the in vitro differentiation protocol display glial cell characteristics and features, the cells can be transplanted into an animal model. In some embodiments, the glial cells are injected into an immunocompromised mouse, e.g., an immunocompromised shiverer mouse. The glial cells are administered to the brain of the mouse and after a pre-selected amount of time the engrafted cells are evaluated. In some instances, the engrafted cells in the brain are visualized by using immunostaining and imaging methods. In some embodiments, it is determined that the glial cells express known glial cell biomarkers.

Useful methods for generating glial cells, precursors, and progenitors thereof from stem cells are found, for example, in U.S. Pat. Nos. 7,579,188; 7,595,194; 8,263,402; 8,206,699; 8,252,586; 9,193,951; 9,862,925; 8,227,247; 9,709,553; US2018/0187148; US2017/0198255; US2017/0183627; US2017/0182097; US2017/253856; US2018/0236004; WO2017/172976; and WO2018/093681.

In some embodiments, differentiation of pluripotent stem cells is performed by exposing or contacting cells to specific factors which are known to produce a specific cell lineage(s), so as to target their differentiation to a specific, desired lineage and/or cell type of interest. In some embodiments, terminally differentiated cells display specialized phenotypic characteristics or features. In certain embodiments, the stem cells described herein are differentiated into a neuroectodermal, neuronal, neuroendocrine, dopaminergic, cholinergic, serotonergic (5-HT), glutamatergic, GABAergic, adrenergic, noradrenergic, sympathetic neuronal, parasympathetic neuronal, sympathetic peripheral neuronal, or glial cell population. In some instances, the glial cell population includes a microglial (e.g., amoeboid, ramified, activated phagocytic, and activated non-phagocytic) cell population or a macroglial (central nervous system cell: astrocyte, oligodendrocyte, ependymal cell, and radial glia; and peripheral nervous system cell: Schwann cell and satellite cell) cell population, or the precursors and progenitors of any of the preceding cells.

Protocols for generating different types of neural cells are described in PCT Application No. WO2010144696, U.S. Pat. Nos. 9,057,053; 9,376,664; and 10,233,422. Additional descriptions of methods for differentiating hypoimmunogenic pluripotent cells can be found, for example, in Deuse et al., Nature Biotechnology, 2019, 37, 252-258 and Han et al., Proc Natl Acad Sci USA, 2019, 116(21), 10441-10446. Methods for determining the effect of neural cell transplantation in an animal model of a neurological disorder or condition are described in the following references: for spinal cord injury—Curtis et al., Cell Stem Cell, 2018, 22, 941-950; for Parkinson's disease—Kikuchi et al., Nature, 2017, 548:592-596; for ALS—Izrael et al., Stem Cell Research, 2018, 9(1):152 and Izrael et al., IntechOpen, DOI: 10.5772/intechopen.72862; for epilepsy—Upadhya et al., PNAS, 2019, 116(1):287-296

The efficacy of neural cell transplants for spinal cord injury can be assessed in, for example, a rat model for acutely injured spinal cord, as described by McDonald, et al., Nat. Med., 1999, 5:1410) and Kim, et al., Nature, 2002, 418:50. For instance, successful transplants may show transplant-derived cells present in the lesion 2-5 weeks later, differentiated into astrocytes, oligodendrocytes, and/or neurons, and migrating along the spinal cord from the lesioned end, and an improvement in gait, coordination, and weight-bearing. Specific animal models are selected based on the neural cell type and neurological disease or condition to be treated.

The neural cells can be administered in a manner that permits them to engraft to the intended tissue site and reconstitute or regenerate the functionally deficient area. For instance, neural cells can be transplanted directly into parenchymal or intrathecal sites of the central nervous system, according to the disease being treated. In some embodiments, any of the neural cells described herein including cerebral endothelial cells, neurons, dopaminergic neurons, ependymal cells, astrocytes, microglial cells, oligodendrocytes, and Schwann cells are injected into a patient by way of intravenous, intraspinal, intracerebroventricular, intrathecal, intraarterial, intramuscular, intraperitoneal, subcutaneous, intramuscular, intra-abdominal, intraocular, retrobulbar and combinations thereof. In some embodiments, the cells are injected or deposited in the form of a bolus injection or continuous infusion. In certain embodiments, the neural cells are administered by injection into the brain, apposite the brain, and combinations thereof. The injection can be made, for example, through a burr hole made in the subject's skull. Suitable sites for administration of the neural cell to the brain include, but are not limited to, the cerebral ventricle, lateral ventricles, cisterna magna, putamen, nucleus basalis, hippocampus cortex, striatum, caudate regions of the brain and combinations thereof.

Additional descriptions of neural cells including dopaminergic neurons for use in the present invention are found in WO2020/018615, the disclosure is herein incorporated by reference in its entirety.

3. Endothelial Cells Differentiated from Pluripotent Stem Cells

The invention provides pluripotent stem cells that may be differentiated into various endothelial cell types for subsequent transplantation or engraftment into subjects (e.g., recipients). As will be appreciated by those in the art, the methods for differentiation depend on the desired cell type using known techniques. Exemplary endothelial cell types include, but are not limited to, a capillary endothelial cell, vascular endothelial cell, aortic endothelial cell, arterial endothelial cell, venous endothelial cell, renal endothelial cell, brain endothelial cell, liver endothelial cell, and the like.

The endothelial cells outlined herein can express one or more endothelial cell markers. Non-limiting examples of such markers include VE-cadherin (CD 144), ACE (angiotensin-converting enzyme) (CD 143), BNH9/BNF13, CD31, CD34, CD54 (ICAM-1), CD62E (E-Selectin), CD105 (Endoglin), CD146, Endocan (ESM-1), Endoglyx-1, Endomucin, Eotaxin-3, EPAS1 (Endothelial PAS domain protein 1), Factor VIII related antigen, FLI-1, Flk-1 (KDR, VEGFR-2), FLT-1 (VEGFR-1), GATA2, GBP-1 (guanylate-binding protein-1), GRO-alpha, HEX, ICAM-2 (intercellular adhesion molecule 2), LM02, LYVE-1, MRB (magic roundabout), Nucleolin, PAL-E (pathologische anatomie Leiden-endothelium), RTKs, sVCAM-1, TALI, TEM1 (Tumor endothelial marker 1), TEM5 (Tumor endothelial marker 5), TEM7 (Tumor endothelial marker 7), Thrombomodulin (TM, CD141), VCAM-1 (vascular cell adhesion molecule-1) (CD106), VEGF (Vascular endothelial growth factor), vWF (von Willebrand factor), ZO-1, endothelial cell-selective adhesion molecule (ESAM), CD102, CD93, CD184, CD304, and DLL4.

In some embodiments, the endothelial cells are genetically modified to express an exogenous gene encoding a protein of interest such as but not limited to an enzyme, hormone, receptor, ligand, or drug that is useful for treating a disorder/condition or ameliorating symptoms of the disorder/condition. Standard methods for genetically modifying endothelial cells are described, e.g., in U.S. Pat. No. 5,674,722.

Such endothelial cells can be used to provide constitutive synthesis and delivery of polypeptides or proteins, which are useful in prevention or treatment of disease. In this way, the polypeptide is secreted directly into the bloodstream or other area of the body (e.g., central nervous system) of the individual. In some embodiments, the endothelial cells can be modified to secrete insulin, a blood clotting factor (e.g., Factor VIII or von Willebrand Factor), alpha-1 antitrypsin, adenosine deaminase, tissue plasminogen activator, interleukins (e.g., IL-1, IL-2, IL-3), and the like.

In certain embodiments, the endothelial cells can be modified in a way that improves their performance in the context of an implanted graft. Non-limiting illustrative examples include secretion or expression of a thrombolytic agent to prevent intraluminal clot formation, secretion of an inhibitor of smooth muscle proliferation to prevent luminal stenosis due to smooth muscle hypertrophy, and expression and/or secretion of an endothelial cell mitogen or autocrine factor to stimulate endothelial cell proliferation and improve the extent or duration of the endothelial cell lining of the graft lumen.

In some embodiments, the engineered endothelial cells are utilized for delivery of therapeutic levels of a secreted product to a specific organ or limb. For example, a vascular implant lined with endothelial cells engineered (transduced) in vitro can be grafted into a specific organ or limb. The secreted product of the transduced endothelial cells will be delivered in high concentrations to the perfused tissue, thereby achieving a desired effect to a targeted anatomical location.

In other embodiments, the endothelial cells are genetically modified to contain a gene that disrupts or inhibits angiogenesis when expressed by endothelial cells in a vascularizing tumor. In some cases, the endothelial cells can also be genetically modified to express any one of the selectable suicide genes described herein which allows for negative selection of grafted endothelial cells upon completion of tumor treatment.

In some embodiments, endothelial cells described herein are administered to a recipient subject to treat a vascular disorder selected from the group consisting of vascular injury, cardiovascular disease, vascular disease, peripheral vascular disease, ischemic disease, myocardial infarction, congestive heart failure, peripheral vascular obstructive disease, hypertension, ischemic tissue injury, reperfusion injury, limb ischemia, stroke, neuropathy (e.g., peripheral neuropathy or diabetic neuropathy), organ failure (e.g., liver failure, kidney failure, and the like), diabetes, rheumatoid arthritis, osteoporosis, cerebrovascular disease, hypertension, angina pectoris and myocardial infarction due to coronary artery disease, renal vascular hypertension, renal failure due to renal artery stenosis, claudication of the lower extremities, other vascular condition or disease.

In some embodiments, the hypoimmunogenic pluripotent cells are differentiated into endothelial colony forming cells (ECFCs) to form new blood vessels to address peripheral arterial disease. Techniques to differentiate endothelial cells are known. See, e.g., Prasain et al., doi: 10.1038/nbt.3048, incorporated herein by reference in its entirety and specifically for the methods and reagents for the generation of endothelial cells from human pluripotent stem cells, and also for transplantation techniques. Differentiation can be assayed as is known in the art, generally by evaluating the presence of endothelial cell associated or specific markers or by measuring functionally.

In some embodiments, the method of producing a population of hypoimmunogenic endothelial cells from a population of hypoimmunogenic pluripotent cells by in vitro differentiation comprises: (a) culturing a population of HIP cells in a first culture medium comprising a GSK inhibitor; (b) culturing the population of HIP cells in a second culture medium comprising VEGF and bFGF to produce a population of pre-endothelial cells; and (c) culturing the population of pre-endothelial cells in a third culture medium comprising a ROCK inhibitor and an ALK inhibitor to produce a population of hypoimmunogenic endothelial cells.

In some embodiments, the GSK inhibitor is CHIR-99021, a derivative thereof, or a variant thereof. In some instances, the GSK inhibitor is at a concentration ranging from about 1 mM to about 10 mM. In some embodiments, the ROCK inhibitor is Y-27632, a derivative thereof, or a variant thereof. In some instances, the ROCK inhibitor is at a concentration ranging from about 1 pM to about 20 pM. In some embodiments, the ALK inhibitor is SB-431542, a derivative thereof, or a variant thereof. In some instances, the ALK inhibitor is at a concentration ranging from about 0.5 pM to about 10 pM.

In some embodiments, the first culture medium comprises from 2 pM to about 10 pM of CHIR-99021. In some embodiments, the second culture medium comprises 50 ng/ml VEGF and 10 ng/ml bFGF. In other embodiments, the second culture medium further comprises Y-27632 and SB-431542. In various embodiments, the third culture medium comprises 10 pM Y-27632 and 1 pM SB-431542. In certain embodiments, the third culture medium further comprises VEGF and bFGF. In particular instances, the first culture medium and/or the second medium is absent of insulin.

The cells of the present invention can be cultured on a surface, such as a synthetic surface to support and/or promote differentiation of hypoimmunogenic pluripotent cells into cardiac cells. In some embodiments, the surface comprises a polymer material including, but not limited to, a homopolymer or copolymer of selected one or more acrylate monomers. Non-limiting examples of acrylate monomers and methacrylate monomers include tetra(ethylene glycol) diacrylate, glycerol dimethacrylate, 1,4-butanediol dimethacrylate, poly(ethylene glycol) diacrylate, di(ethylene glycol) dimethacrylate, tetra(ethyiene glycol) dimethacrylate, 1,6-hexanediol propoxylate diacrylate, neopentyl glycol diacrylate, trimethylolpropane benzoate diacrylate, trimethylolpropane eihoxylate (1 EO/QH) methyl, tricyclo[5.2.1.0^{2,6}] decane dimethanol diacrylate, neopentyl glycol exhoxylate diacrylate, and trimethylolpropane triacrylate. Acrylate synthesized as known in the art or obtained from a commercial vendor, such as Polysciences, Inc., Sigma Aldrich, Inc. and Sartomer, Inc.

In some embodiments, the endothelial cells may be seeded onto a polymer matrix. In some cases, the polymer matrix is biodegradable. Suitable biodegradable matrices are well known in the art and include collagen-GAG, collagen, fibrin, PLA, PGA, and PLA/PGA copolymers. Additional biodegradable materials include poly(anhydrides), poly(hydroxy acids), poly(ortho esters), poly(propylfumerates), poly(caprolactones), polyamides, polyamino acids, polyacetals, biodegradable polycyanoacrylates, biodegradable polyurethanes and polysaccharides.

Non-biodegradable polymers may also be used as well. Other non-biodegradable, yet biocompatible polymers include polypyrrole, polyanibnes, polythiophene, polystyrene, polyesters, non-biodegradable polyurethanes, polyureas, poly(ethylene vinyl acetate), polypropylene, polymethacrylate, polyethylene, polycarbonates, and poly(ethylene oxide). The polymer matrix may be formed in any shape, for example, as particles, a sponge, a tube, a sphere, a strand, a coiled strand, a capillary network, a film, a fiber, a mesh, or a sheet. The polymer matrix can be modified to include natural or synthetic extracellular matrix materials and factors.

The polymeric material can be dispersed on the surface of a support material. Useful support materials suitable for culturing cells include a ceramic substance, a glass, a plastic, a polymer or co-polymer, any combinations thereof, or a coating of one material on another. In some instances, a glass includes soda-lime glass, pyrex glass, vycor glass, quartz glass, silicon, or derivatives of these or the like.

In some instances, plastics or polymers including dendritic polymers include poly(vinyl chloride), poly(vinyl alcohol), poly(methyl methacrylate), poly(vinyl acetate-maleic anhydride), poly(dimethylsiloxane) monomethacrylate, cyclic olefin polymers, fluorocarbon polymers, polystyrenes, polypropylene, polyethyleneimine or derivatives of these or the like. In some instances, copolymers include poly(vinyl acetate-co-maleic anhydride), poly(styrene-co-maleic anhydride), poly(ethylene-co-acrylic acid) or derivatives of these or the like.

In some embodiments, the population of hypoimmunogenic endothelial cells is isolated from non-endothelial cells. In some embodiments, the isolated population of hypoimmunogenic endothelial cells are expanded prior to administration. In certain embodiments, the isolated population of hypoimmunogenic endothelial cells are expanded and cryopreserved prior to administration.

Additional descriptions of endothelial cells for use in the present invention are found in WO2020/018615, the disclosure is herein incorporated by reference in its entirety.

4. Thyroid Cells Differentiated from Pluripotent Stem Cells

In some embodiments, the pluripotent stem cells may be differentiated into thyroid progenitor cells and thyroid follicular organoids that can secrete thyroid hormones to address autoimmune thyroiditis. Techniques to differentiate thyroid cells are known the art. See, e.g. Kurmann et al., Cell Stem Cell, 2015 Nov. 5; 17(5):527-42, incorporated herein by reference in its entirety and specifically for the methods and reagents for the generation of thyroid cells from human pluripotent stem cells, and also for transplantation techniques. Differentiation can be assayed as is known in the art, generally by evaluating the presence of thyroid cell associated or specific markers or by measuring functionally.

5. Hepatocytes Differentiated from Pluripotent Stem Cells

In some embodiments, the pluripotent stem cells may be differentiated into hepatocytes to address loss of the hepatocyte functioning or cirrhosis of the liver. There are a number of techniques that can be used to differentiate HIP cells into hepatocytes; see for example Pettinato et al, doi: 10.1038/spre32888, Snykers et al, Methods Mol Biol 698:305-314 (2011), Si-Tayeb et al, Hepatology 51:297-305 (2010) and Asgari et al, Stem Cell Rev (:493-504 (2013), all of which are incorporated herein by reference in their entirety and specifically for the methodologies and reagents for differentiation. Differentiation is assayed as is known in the art, generally by evaluating the presence of hepatocyte associated and/or specific markers, including, but not limited to, albumin, alpha fetoprotein, and fibrinogen. Differentiation can also be measured functionally, such as the metabolization of ammonia, LDL storage and uptake, ICG uptake and release and glycogen storage.

6. Pancreatic Islet Cells Differentiated from Pluripotent Stem Cells

The invention provides pluripotent stem cells that may be differentiated into various pancreatic islet cell types for subsequent transplantation or engraftment into subjects (e.g., recipients). As will be appreciated by those in the art, the methods for differentiation depend on the desired cell type using known techniques. Exemplary pancreatic islet cell types include, but are not limited to, pancreatic islet progenitor cell, immature pancreatic islet cell, mature pancreatic islet cell, and the like. In some embodiments, pancreatic cells described herein are administered to a subject to treat diabetes.

In some embodiments, pancreatic islet cells are derived from the hypoimmunogenic pluripotent cells described herein. Useful method for differentiating pluripotent stem cells into pancreatic islet cells are described, for example, in U.S. Pat. Nos. 9,683,215; 9,157,062; and 8,927,280.

In some embodiments, the pancreatic islet cells produced by the methods as disclosed herein secretes insulin. In some embodiments, a pancreatic islet cell exhibits at least two characteristics of an endogenous pancreatic islet cell, for example, but not limited to, secretion of insulin in response to glucose, and expression of beta cell markers.

Exemplary beta cell markers or beta cell progenitor markers include, but are not limited to, c-peptide, Pdxl, glucose transporter 2 (Glut2), HNF6, VEGF, glucokinase (GCK), prohormone convertase (PC 1/3), Cdcpl, NeuroD, Ngn3, Nkx2.2, Nkx6.1, Nkx6.2, Pax4, Pax6, Ptfla, Isll, Sox9, Sox17, and FoxA2.

In some embodiments, the isolated pancreatic islet cells produce insulin in response to an increase in glucose. In various embodiments, the isolated pancreatic islet cells secrete insulin in response to an increase in glucose. In some embodiments, the cells have a distinct morphology such as a cobblestone cell morphology and/or a diameter of about 17 pm to about 25 pm.

In some embodiments, the hypoimmunogenic pluripotent cells are differentiated into beta-like cells or islet organoids for transplantation to address type I diabetes mellitus (T1DM). Cell systems are a promising way to address T1DM, see, e.g., Ellis et al, Nat Rev Gastroenterol Hepatol. 2017 October; 14(10):612-628, incorporated herein by reference. Additionally, Pagliuca et al. (Cell, 2014, 159(2):428-39) reports on the successful differentiation of β-cells from hiPSCs, the contents incorporated herein by reference in its entirety and in particular for the methods and reagents outlined there for the large-scale production of functional human β cells from human pluripotent stem cells). Furthermore, Vegas et al. shows the production of human β cells from human pluripotent stem cells followed by encapsulation to avoid immune rejection by the host; Vegas et al., Nat Med, 2016, 22(3):306-11, incorporated herein by reference in its entirety and in particular for the methods and reagents outlined there for the large-scale production of functional human β cells from human pluripotent stem cells.

In some embodiments, the method of producing a population of hypoimmunogenic pancreatic islet cells from a population of hypoimmunogenic pluripotent cells by in vitro differentiation comprises: (a) culturing the population of HIP cells in a first culture medium comprising one or more factors selected from the group consisting insulin-like growth factor (IGF), transforming growth factor (TGF), fibroblast growth factor (EGF), epidermal growth factor (EGF), hepatocyte growth factor (HGF), sonic hedgehog (SHH), and vascular endothelial growth factor (VEGF), transforming growth factor-b (TORb) superfamily, bone morphogenic protein-2 (BMP2), bone morphogenic protein-7 (BMP7), a GSK inhibitor, an ALK inhibitor, a BMP type 1 receptor inhibitor, and retinoic acid to produce a population of immature pancreatic islet cells; and (b) culturing the population of immature pancreatic islet cells in a second culture medium that is different than the first culture medium to produce a population of hypoimmune pancreatic islet cells. In some embodiments, the GSK inhibitor is CHIR-99021, a derivative thereof, or a variant thereof. In some instances, the GSK inhibitor is at a concentration ranging from about 2 mM to about 10 mM. In some embodiments, the ALK inhibitor is SB-431542, a derivative thereof, or a variant thereof. In some instances, the ALK inhibitor is at a concentration ranging from about 1 pM to about 10 pM. In some embodiments, the first culture medium and/or second culture medium are absent of animal serum.

In some embodiments, the population of hypoimmunogenic pancreatic islet cells is isolated from non-pancreatic islet cells. In some embodiments, the isolated population of hypoimmunogenic pancreatic islet cells are expanded prior to administration. In certain embodiments, the isolated population of hypoimmunogenic pancreatic islet cells are expanded and cryopreserved prior to administration.

Differentiation is assayed as is known in the art, generally by evaluating the presence of β cell associated or specific markers, including but not limited to, insulin. Differentiation can also be measured functionally, such as measuring glucose metabolism, see generally Muraro et al., Cell Syst. 2016 Oct. 26; 3(4): 385-394.e3, hereby incorporated by reference in its entirety, and specifically for the biomarkers outlined there. Once the beta cells are generated, they can be transplanted (either as a cell suspension or within a gel matrix as discussed herein) into the portal vein/liver, the omentum, the gastrointestinal mucosa, the bone marrow, a muscle, or subcutaneous pouches.

Additional descriptions of pancreatic islet cells including dopaminergic neurons for use in the present invention are found in WO2020/018615, the disclosure is herein incorporated by reference in its entirety.

7. Retinal Pigmented Epithelium (RPE) Cells Differentiated from Pluripotent Stem Cells

The invention provides hypoimmunogenic pluripotent cells that may be differentiated into various RPE cell types for subsequent transplantation or engraftment into subjects (e.g., recipients). As will be appreciated by those in the art, the methods for differentiation depend on the desired cell type using known techniques. Exemplary RPE cell types include, but are not limited to, retinal pigmented epithelium (RPE) cell, RPE progenitor cell, immature RPE cell, mature RPE cell, functional RPE cell, and the like.

Useful methods for differentiating pluripotent stem cells into RPE cells are described in, for example, U.S. Pat. Nos. 9,458,428 and 9,850,463, the disclosures are herein incorporated by reference in their entirety, including the specifications. Additional methods for producing RPE cells from human induced pluripotent stem cells can be found in, for example, Lamba et al., PNAS, 2006, 103(34): 12769-12774; Mellough et al, Stem Cells, 2012, 30(4):673-686; Idelson et al, Cell Stem Cell, 2009, 5(4): 396-408; Rowland et al, Journal of Cellular Physiology, 2012, 227(2):457-466, Buchholz et al, Stem Cells Trans Med, 2013, 2(5): 384-393, and da Cruz et al, Nat Biotech, 2018, 36:328-337.

In some embodiments, RPE cells described herein are administered to a subject to treat an eye disorder selected from the group consisting of wet macular degeneration, dry macular degeneration, juvenile macular degeneration (e.g., Stargardt disease, Best disease, and juvenile retinoschisis), Leber's Congenital Ameurosis, retinitis pigmentosa, retinal detachment, age-related macular degeneration (AMD), early AMD, intermediate AMD, late AMD, non-neovascular age-related macular degeneration, and the like.

Human pluripotent stem cells have been differentiated into RPE cells using the techniques outlined in Kamao et al, Stem Cell Reports 2014:2:205-18, hereby incorporated by reference in its entirety and in particular for the methods and reagents outlined there for the differentiation techniques and reagents; see also Mandai et al., N Engl J Med, 2017, 376:1038-1046, the contents herein incorporated in its entirety for techniques for generating sheets of RPE cells and transplantation into patients. Differentiation can be assayed as is known in the art, generally by evaluating the presence of RPE associated and/or specific markers or by measuring functionally. See for example Kamao et al., Stem Cell Reports, 2014, 2(2):205-18, the contents incorporated herein by reference in its entirety and specifically for the markers outlined in the first paragraph of the results section.

In some embodiments, the method of producing a population of hypoimmunogenic retinal pigmented epithelium (RPE) cells from a population of hypoimmunogenic pluripotent cells by in vitro differentiation comprises: (a) culturing the population of hypoimmunogenic pluripotent cells in a first culture medium comprising any one of the factors selected from the group consisting of activin A, bFGF, BMP4/7, DKK1, IGF1, noggin, a BMP inhibitor, an ALK inhibitor, a ROCK inhibitor, and a VEGFR inhibitor to produce a population of pre-RPE cells; and (b) culturing the population of pre-RPE cells in a second culture medium that is different than the first culture medium to produce a population of hypoimmunogenic RPE cells. In some embodiments, the ALK inhibitor is SB-431542, a derivative thereof, or a variant thereof. In some instances, the ALK inhibitor is at a concentration ranging from about 2 mM to about 10 pM. In some embodiments, the ROCK inhibitor is Y-27632, a derivative thereof, or a variant thereof. In some instances, the ROCK inhibitor is at a concentration ranging from about 1 pM to about 10 pM. In some embodiments, the first culture medium and/or second culture medium are absent of animal serum.

Differentiation can be assayed as is known in the art, generally by evaluating the presence of RPE associated and/or specific markers or by measuring functionally. See for example Kamao et al., Stem Cell Reports, 2014, 2(2):205-18, the contents are herein incorporated by reference in its entirety and specifically for the results section.

Additional descriptions of RPE cells for use in the present invention are found in WO2020/018615, the disclosure is herein incorporated by reference in its entirety.

For therapeutic application, cells prepared according to the disclosed methods can typically be supplied in the form of a pharmaceutical composition comprising an isotonic excipient, and are prepared under conditions that are sufficiently sterile for human administration. For general principles in medicinal formulation of cell compositions, see “Cell Therapy: Stem Cell Transplantation, Gene Therapy, and Cellular Immunotherapy,” by Morstyn & Sheridan eds, Cambridge University Press, 1996; and “Hematopoietic Stem Cell Therapy,” E. D. Ball, J. Lister & P. Law, Churchill Livingstone, 2000. The cells can be packaged in a device or container suitable for distribution or clinical use.

### P. Administration of Cells

As will be appreciated by those in the art, the differentiated hypoimmunogenic pluripotent cell derivatives can be transplanted using techniques known in the art that depends on both the cell type and the ultimate use of these cells. In general, the cells of the invention can be transplanted either intravenously or by injection at particular locations in the patient. When transplanted at particular locations, the cells may be suspended in a gel matrix to prevent dispersion while they take hold.

### IV. Examples

### Example 1

The effect of CD24 on macrophage engulfment of CD24-expressing cells is measured using XCELLIGENCE assay. Briefly, human B2M^{-/-}CIITA^{-/-} iPSCs transduced with lentiviral vector expressing CD24 (CD24 tg) or untransduced are cultured on diluted feeder-free matrigel (hESC qualified, BD Biosciences, San Jose, Calif.)-coated 10 cm dishes in Essential 8 Flex medium (Thermo Fisher Scientific). Medium is changed every 24 hours, and Versene (Gibco) is used for cell passaging at a ratio of 1:6. Differentiation to endothelial cells is initiated at 60% confluency, and medium is changed to RPMI-1640 containing 2% B-27 minus insulin (both Gibco) and 5 μM CHIR-99021 (Selleckchem). On day 2, the medium is changed to reduced medium: RPMI-1640 containing 2% B-27 minus insulin (Gibco) and 2 μM CHIR-99021. From culture day 4 to 7, the cells are exposed to RPMI-1640 EC medium, RPMI-1640 containing 2% B-27 minus insulin plus 50 ng/ml human vascular endothelial growth factor (VEGF; R&D Systems), 10 ng/ml human fibroblast growth factor basic (FGFb; R&D Systems), 10 μM Y-27632 (Sigma-Aldrich), and 1 μM SB 431542 (Sigma-Aldrich). Endothelial cell clusters are visible from day 7 and cells are maintained in Endothelial Cell Basal Medium 2 (PromoCell, Heidelberg, Germany) plus supplements, 10% FCS hi (Gibco), 1% pen/strep, 25 ng/ml VEGF, 2 ng/ml FGFb, 10 μM Y-27632 (Sigma-Aldrich), and 1 μM SB 431542 (Sigma-Aldrich). The differentiation protocol is completed after 14 days; and undifferentiated cells detach during the differentiation process. TRYPLE EXPRESS (Gibco) is used for passaging the cells 1:3 every 3 to 4 days.

NK cell killing and macrophage killing assays are performed on the XCELLIGENCE MP platform (ACEA BioSciences). Special 96-well E-plates are coated with gelatin (Millipore) and 4×10⁵ B2M^{-/-}CIITA^{-/-} CD24 tg or 4×10⁵ B2M^{-/-}CIITA^{-/-} hiECs are plated in 100 μl cell-specific medium. After the cell index value reaches 0.7, human NK cells or macrophages are added at an effector cell-to-target cell (E:T) ratio of 1:1 with 1 ug/ml human IL-2 (PeproTech). As a negative control, cells are treated with 2% Triton X-100. Data are standardized and analyzed with the RTCA software (ACEA BioSciences). Whereas the B2M^{-/-}CIITA^{-/-} hiECs (without CD24) are effectively killed by NK cells and macrophages, the B2M^{-/-}CIITA^{-/-} CD24 tg hiECs are protected from killing by macrophages. Blockade with 10 ug/ml of an anti-CD24 antibody (Clone SN3, Novus Biologics) removed the protective effect. Overexpression of both CD47 and CD24 protects B2M^{-/-}CIITA^{-/-} hiECs from killing by both NK cells and macrophages.

### Example 2

Macrophage phagocytosis is also measure using flow cytometry. Human B2M^{-/-}CIITA^{-/-} iPSCs transduced with lentiviral vector expressing CD24 (CD24 tg) or untransduced are cultured on diluted feeder-free MATRIGEL (hESC qualified, BD Biosciences, San Jose, Calif.)-coated 10 cm dishes in Essential 8 Flex medium (Thermo Fisher Scientific). The cells are harvested at 60% confluency and fluorescently labelled with Calcein AM (Invitrogen) by suspending cells in PBS+1:30,000 Calcein AM as per the manufacturer's instructions for 15 minutes at 37° C. and washed twice with 40 ml PBS before co-culture. The cells are then co-cultured at an effector-to-target cell (E:T) ratio of 1:2 with human macrophages stimulated for 4 days with 50 ng/ml human TGFβ1 and 50 ng/ml human IL-10. After co-culture, phagocytosis assays are stopped by placing plates on ice, centrifuged at 400 g for 5 minutes at 4° C. and stained with A647-labelled anti-CD11b (Clone M1/70, BioLegend) to identify human macrophages. Assays are analyzed by flow cytometry on an Attune NxT flow analyzer. Phagocytosis is measured as the number of CD11b+calcneurin+ macrophages, quantified as a percentage of the total CD11b+macrophages. Whereas the B2M^{-/-}CIITA^{-/-} hiECs (without CD24) are significantly phagocytosed, the B2M^{-/-}CIITA^{-/-} CD24 tg hiECs are protected from phagocytosis. Blockade with 10 ug/ml of an anti-CD24 antibody (Clone SN3, Novus Biologics) removes the protective effect. Macrophage phagocytosis is also measure using flow cytometry. Human B2M^{-/-}CIITA^{-/-} iPSCs transduced with lentiviral vector expressing CD24 (CD24 tg) or untransduced are cultured on diluted feeder-free MATRIGEL (hESC qualified, BD Biosciences, San Jose, Calif.)-coated 10 cm dishes in Essential 8 Flex medium (Thermo Fisher Scientific). The cells are harvested at 60% confluency and fluorescently labelled with Calcein AM (Invitrogen) by suspending cells in PBS+1:30,000 Calcein AM as per the manufacturer's instructions for 15 minutes at 37° C. and washed twice with 40 ml PBS before co-culture. The cells are then co-cultured at an effector-to-target cell (E:T) ratio of 1:2 with human macrophages stimulated for 4 days with 50 ng/ml human TGFβ1 and 50 ng/ml human IL-10. After co-culture, phagocytosis assays are stopped by placing plates on ice, centrifuged at 400 g for 5 minutes at 4° C. and stained with A647-labelled anti-CD11b (Clone M1/70, BioLegend) to identify human macrophages. Assays are analyzed by flow cytometry on an Attune NxT flow analyzer. Phagocytosis is measured as the number of CD11b+calcneurin+ macrophages, quantified as a percentage of the total CD11b+macrophages. Whereas the B2M^{-/-}CIITA^{-/-} hiECs (without CD24) are significantly phagocytosed, the B2M^{-/-}CIITA^{-/-} CD24 tg hiECs are protected from phagocytosis. Blockade with 10 ug/ml of an anti-CD24 antibody (Clone SN3, Novus Biologics) removes the protective effect.

All headings and section designations are used for clarity and reference purposes only and are not to be considered limiting in any way. For example, those of skill in the art will appreciate the usefulness of combining various aspects from different headings and sections as appropriate according to the spirit and scope of the invention described herein.

All references cited herein are hereby incorporated by reference herein in their entireties and for all purposes to the same extent as if each individual publication or patent or patent application was specifically and individually indicated to be incorporated by reference in its entirety for all purposes.

Many modifications and variations of this application can be made without departing from its spirit and scope, as will be apparent to those skilled in the art. The specific embodiments and examples described herein are offered by way of example only, and the application is to be limited only by the terms of the appended claims, along with the full scope of equivalents to which the claims are entitled.

1. An isolated cell comprising reduced expression of WIC class I and/or WIC class II human leukocyte antigens and a modification to increase expression of CD24 in the cell.

2. The isolated cell of claim 1, wherein the cell comprises reduced expression of MHC class I and MHC class II human leukocyte antigens.

3. The isolated cell of claim 1, wherein the cell further comprises:
a. a genetic modification targeting a CIITA gene by a rare-cutting endonuclease that selectively inactivates the CIITA gene; and/or
b. a modification to increase expression of a polypeptide selected from the group consisting of CD47, DUX4, CD27, CD35, CD46, CD55, CD59, CD200, HLA-C, HLA-E, HLA-E heavy chain, HLA-G, PD-L1, IDO1, CTLA4-Ig, C1-Inhibitor, IL-10, IL-35, FASL, CCL21, Mfge8, and Serpinb9 in the cell.

4. (canceled)

5. The isolated cell of claim 3, wherein the cell further comprises:
a. a modification to increase expression of CD47 in the cell;
b. a genetic modification targeting a B2M gene by a rare-cutting endonuclease that selectively inactivates the B2M gene; and/or
c. a genetic modification targeting an NLRC5 gene by a rare-cutting endonuclease that selectively inactivates the NLRC5 gene.

6.-7. (canceled)

8. The isolated cell of claim 1, wherein the rare-cutting endonuclease is selected from the group consisting of a Cas protein, a TALE-nuclease, a zinc finger nuclease, a meganuclease, and a homing nuclease.

9. The isolated cell of claim 3, wherein the genetic modification targeting the CIITA gene by the rare-cutting endonuclease comprises a Cas protein or a polynucleotide encoding a Cas protein, and at least one guide ribonucleic acid sequence for specifically targeting the CIITA gene.

10. The isolated cell of claim 5, wherein:
a. the genetic modification targeting the B2M gene by the rare-cutting endonuclease comprises a Cas protein or a polynucleotide encoding a Cas protein, and at least one guide ribonucleic acid sequence for specifically targeting the B2M gene; and/or
b. the genetic modification targeting the NLRC5 gene by the rare-cutting endonuclease comprises a Cas protein or a polynucleotide encoding a Cas protein, and at least one guide ribonucleic acid sequence for specifically targeting the NLRC5 gene.

11. (canceled)

12. The isolated cell of claim 1, wherein the modification to increase expression of CD24 comprises introducing an expression vector comprising a polynucleotide sequence encoding CD24 into the cell.

13. The isolated cell of claim 12, wherein:
a. the polynucleotide sequence encoding CD24 is a nucleotide sequence encoding a polypeptide sequence having at least 95% sequence identity to a sequence selected from the group consisting of SEQ ID NOS:28-31; or
the polynucleotide sequence encoding CD24 is a nucleotide sequence encoding a polypeptide having a sequence selected from the group consisting of SEQ ID NOS:28-31.

14.-18. (canceled)

19. The isolated cell of claim 1, wherein the modification to increase expression of CD24 comprises introducing a polynucleotide sequence encoding CD24 into a selected locus of the cell.

20. The isolated cell of claim 19, wherein the polynucleotide sequence encoding CD24 is a nucleotide sequence encoding a polypeptide sequence having at least 95% sequence identity to a sequence selected from the group consisting of SEQ ID NOS:28-31, or wherein the polynucleotide sequence encoding CD24 is a nucleotide sequence encoding a polypeptide having a sequence selected from the group consisting of SEQ ID NOS:28-31.

21. (canceled)

22. The isolated cell of claim 3, wherein the modification to increase expression of one or more polypeptides selected from the group consisting of CD47, CD35, DUX4, HLA-C, HLA-E, HLA-G, PD-L1, CTLA4, C1-inhibitor, CD46, CD55, CD59, and IL-35 comprises introducing a polynucleotide sequence encoding the one or more polypeptides selected from the group consisting of CD47, CD35, DUX4, HLA-C, HLA-E, HLA-G, PD-L1, CTLA4, C1-inhibitor, CD46, CD55, CD59, and IL-35 optionally into a selected locus of the cell.

23. The isolated cell of claim 22, wherein the modification to increase expression of CD47 comprises introducing a polynucleotide sequence encoding CD47 into a selected locus of the cell.

24. The isolated cell of claim 19, wherein the selected locus for the polynucleotide sequence encoding CD24 and/or the selected locus for the polynucleotide sequence encoding one selected from the group consisting of CD47, CD35, DUX4, HLA-C, HLA-E, HLA-G, PD-L1, CTLA4, C1-inhibitor, CD46, CD55, CD59, and IL-35 is a safe harbor locus.

25. The isolated cell of claim 24, wherein the safe harbor is selected from the group consisting of an AAVS1 locus, CCRS locus, CLYBL locus, ROSA26 locus, and SHS231 locus.

26. The isolated cell of claim 1, further comprises an inducible suicide switch.

27. The isolated cell of claim 1, wherein the cell is selected from the group consisting of a stem cell, a differentiated cell, a pluripotent stem cell, an induced pluripotent stem cell, an adult stem cell, a progenitor cell, a somatic cell, a primary T cell and a chimeric antigen receptor T cell.

28. A method of preparing a cell comprising CD24, the method comprises introducing:
a. an expression vector comprising a polynucleotide sequence encoding CD24 into the cell, thereby producing the cell comprising CD24; or
b. a polynucleotide sequence encoding CD24 into a selected locus of the stem cell, thereby producing a hypoimmunogenic stem cell.

29.-61. (canceled)

62. A method of treating a patient in need of cell therapy comprising administering a population of differentiated hypoimmunogenic cells prepared according to the method of claim 28.

63. A cell that:
i) expresses CD24. and has reduced expression of MHC class I human leukocyte antigens,
ii) expresses CD24, and has reduced expression of WIC class I and/or WIC class II human leukocyte antigens,
iii) does not express CIITA, expresses CD24, and has reduced expression of WIC class I and/or WIC class II human leukocyte antigen,
iv) does not express B2M, expresses CD24, and has reduced expression of WIC class I and/or MHC class II human leukocyte antigens,
v) does not express NLRC5, expresses CD24, and has reduced expression of MHC class I and/or WIC class II human leukocyte antigens,
vi) expresses CD24 and at least one polypeptide selected from the group consisting of CD47, DUX4, HLA-C, HLA-E, HLA-G, PD-L1, CTLA4, C1-inhibitor, CD46, CD55, CD59, and IL-35, and has reduced expression of WIC class I and/or MHC class II human leukocyte antigens,
vii) expresses CD24 and CD47, and has reduced expression of WIC class I and/or WIC class II human leukocyte antigens,
viii) does not express CIITA, expresses CD24 and at least one polypeptide selected from the group consisting of CD47, DUX4, HLA-C, HLA-E, HLA-G, PD-L1, CTLA4, C1-inhibitor, CD46, CD55, CD59, and IL-35, and has reduced expression of MHC class I and/or MHC class II human leukocyte antigens,
ix) does not express CIITA, expresses CD24 and CD47, and has reduced expression of WIC class I and/or WIC class II human leukocyte antigens,
x)) does not express CIITA and B2M, expresses CD24, and has reduced expression of WIC class I and/or WIC class II human leukocyte antigens,
xi) does not express CIITA and B2M, expresses CD24 and at least one polypeptide selected from the group consisting of CD47, DUX4, HLA-C, HLA-E, HLA-G, PD-L1, CTLA4, C1-inhibitor, CD46, CD55, CD59, and IL-35, and has reduced expression of WIC class I and/or WIC class II human leukocyte antigens,
xii) does not express CIITA and B2M, expresses CD24 and CD47, and has reduced expression of MHC class I and/or MEW class II human leukocyte antigens,
xiii) does not express CIITA and NLRC5, expresses CD24, and has reduced expression of MHC class I and/or MHC class II human leukocyte antigens,
xiv) does not express CIITA and NLRC5, expresses CD24 and at least one polypeptide selected from the group consisting of CD47, CD35, DUX4, HLA-C, HLA-E, HLA-G, PD-L1, CTLA4, C1-inhibitor, CD46, CD55, CD59, and IL-35, and has reduced expression of MHC class I and/or MHC class II human leukocyte antigens,
xv) does not express CIITA and NLRC5, expresses CD24 and CD47, and has reduced expression of MHC class I and/or WIC class II human leukocyte antigens,
xvi) does not express CIITA, B2M, and NLRC5, expresses CD24 and at least one polypeptide selected from the group consisting of CD47, CD35, DUX4, HLA-C, HLA-E, HLA-G, PD-L1, CTLA4, C1-inhibitor, CD46, CD55, CD59, and IL-35, and has reduced expression of WIC class I and/or MHC class II human leukocyte antigens, or
xvii) does not express CIITA, B2M, and NLRC5, expresses CD24 and CD47, and has reduced expression of MHC class I and/or WIC class II human leukocyte antigens.

64.-113. (canceled)

114. A method of preparing a stem cell comprising an exogenous CD24 polypeptide, the method comprising introducing an expression vector comprising a nucleotide sequence encoding a CD24 polypeptide having at least 95% sequence identity to a sequence selected from the group consisting of SEQ ID NO:28, SEQ ID NO:29, SEQ ID NO:30, and SEQ ID NO:31.

115.-147. (canceled)

148. A stem cell expressing:
i) an exogenous CD24 polypeptide and a reduced expression level of MHC class I human leukocyte antigens,
ii) an exogenous CD24 polypeptide and a reduced expression level of MHC class II human leukocyte antigens,
iii an exogenous CD24 polypeptide and a reduced expression level of MHC class I and class II human leukocyte antigens,
iv) an exogenous CD24 polypeptide and a reduced expression level of CIITA,
v) an exogenous CD24 polypeptide and a reduced expression level of B2M
vi) expressing an exogenous CD24 polypeptide and a reduced expression level of NLRC5,
vii) expressing an exogenous CD24 polypeptide and reduced expression levels of CIITA, B2M, NLRC5, and a combination thereof,
viii) expressing an exogenous CD24 polypeptide and one or more tolerogenic factors selected from the group consisting of HLA-C, HLA-E, HLA-G, PD-L1, CTLA-4-Ig, C1-inhibitor, and IL-35,
ix) expressing an exogenous CD24 polypeptide, one or more tolerogenic factors selected from the group consisting of HLA-C, HLA-E, HLA-G, PD-L1, CTLA-4-Ig, C1-inhibitor, and IL-35, and a reduced expression level of CIITA,
x) expressing an exogenous CD24 polypeptide, one or more tolerogenic factors selected from the group consisting of HLA-C, HLA-E, HLA-G, PD-L1, CTLA-4-Ig, C1-inhibitor, and IL-35, and a reduced expression level of B2M,
xo) expressing an exogenous CD24 polypeptide, one or more tolerogenic factors selected from the group consisting of HLA-C, HLA-E, HLA-G, PD-L1, CTLA-4-Ig, C1-inhibitor, and IL-35, and a reduced expression level of NLRC5, or
xii) exogenous CD24 polypeptide, one or more tolerogenic factors selected from the group consisting of HLA-C, HLA-E, HLA-G, PD-L1, CTLA-4-Ig, C1-inhibitor, and IL-35, and reduced expression levels of CIITA, B2M, NLRC5, and a combination thereof.

149.-159. (canceled)

160. A differentiated cell:
i) generated from a stem cell expressing an exogenous CD24 polypeptide and a reduced expression level of MHC class I human leukocyte antigens,
ii) generated from stem cell expressing an exogenous CD24 polypeptide and a reduced expression level of MHC class II human leukocyte antigens,
iii) generated from a stem cell expressing an exogenous CD24 polypeptide and a reduced expression level of MHC class I and class II human leukocyte antigens,
iv) generated from a stem cell expressing an exogenous CD24 polypeptide and a reduced expression level of CIITA,
v) generated from a stem cell expressing an exogenous CD24 polypeptide and a reduced expression level of B2M,
vi) generated from a stem cell expressing an exogenous CD24 polypeptide and a reduced expression level of NLRC5,
vii) generated from a stem cell expressing an exogenous CD24 polypeptide and reduced expression levels of CIITA, B2M, NLRC5, and a combination thereof,
viii) generated from a stem cell expressing an exogenous CD24 polypeptide and one or more tolerogenic factors selected from the group consisting of HLA-C, HLA-E, HLA-G, PD-L1, CTLA-4-Ig, C1-inhibitor, and IL-35,
ix) from a stem cell expressing an exogenous CD24 polypeptide, one or more tolerogenic factors selected from the group consisting of HLA-C, HLA-E, HLA-G, PD-L1, CTLA-4-Ig, C1-inhibitor, and IL-35, and a reduced expression level of CIITA,
x) generated from a stem cell expressing an exogenous CD24 polypeptide, one or more tolerogenic factors selected from the group consisting of HLA-C, HLA-E, HLA-G, PD-L1, CTLA-4-Ig, C1-inhibitor, and IL-35, and a reduced expression level of B2M,
xi) generated from a stem cell expressing an exogenous CD24 polypeptide, one or more tolerogenic factors selected from the group consisting of HLA-C, HLA-E, HLA-G, PD-L1, CTLA-4-Ig, C1-inhibitor, and IL-35, and a reduced expression level of NLRC5, or
xii) generated from a stem cell expressing an exogenous CD24 polypeptide, one or more tolerogenic factors selected from the group consisting of HLA-C, HLA-E, HLA-G, PD-L1, CTLA-4-Ig, C1-inhibitor, and IL-35, and reduced expression levels of CIITA, B2M, NLRC5, and a combination thereof.

161.-173. (canceled)
